(19) United States
(12) Patent Application Publication
(10) Pub. No.: US 2024/0228980 A1
(43) Pub. Date: Jul. 11, 2024

(54) CRYPTIC PROTEINS EXPRESSED FROM DEFECTIVE VIRAL GENOMES INTERFERE WITH INFLUENZA VIRUS REPLICATION

(71) Applicant: Wisconsin Alumni Research Foundation, Madison, WI (US)

(72) Inventors: Andrew Mehle, Madison, WI (US); Jordan Ranum, Waunakee, WI (US); Mitchell Ledwith, San Diego, CA (US)

(21) Appl. No.: 18/495,338

(22) Filed: Oct. 26, 2023

Related U.S. Application Data
(60) Provisional application No. 63/381,105, filed on Oct. 26, 2022.

(51) Int. Cl. C12N 7/00; A61P 31/16

(52) U.S. Cl. CPC C12N 7/00; A61P 31/16; C12N 2760/16132; C12N 2760/16232; C12N 2760/16332

(57) ABSTRACT

The disclosure provides for methods for making and using modified influenza gene products, alone or in combination, e.g., to inhibit wild-type influenza virus replication, to serve as an immunostimulatory agent, and/or as attenuated vaccine backbones. In one embodiment, the genomes of the DIPs provide for inhibitory activity, producing a dual effect in which both the RNA itself and the encoded protein coordinate to interfere with replication. Thus, the ability of DIPs to block replication of WT virus provides for a treatment for infection, use as an immunostimulatory agent, and as attenuated viruses for vaccination.

### CROSS REFERENCE TO RELATED APPLICATIONS

This application is a continuation of U.S. Provisional Application Ser. No. 63/381,105, filed Oct. 26, 2022, the contents of which are specifically incorporated herein by reference in its entirety.

### STATEMENT OF GOVERNMENT SUPPORT

This invention was made with government support under AI125271 and AI164690 awarded by the National Institutes of Health. The government has certain rights in the invention.

### INCORPORATION BY REFERENCE OF SEQUENCE LISTING

This application contains a Sequence Listing which has been submitted electronically in ST26 format and is hereby incorporated by reference in its entirety. Said ST26 file, created on Mar. 12, 2024, is named “800131US1.xml” and is 82,049 bytes in size.

### BACKGROUND

Cells do not have the appropriate machinery to copy RNA viral genomes or transcribe mRNA from an RNA template like that of the influenza virus genome. Therefore, such viruses must carry their own RNA-dependent RNA polymerase. Viral RNA-dependent RNA polymerases have high error rates, sacrificing fidelity for the ability to rapidly replicate viral genomes. In addition to introducing individual mutations, many viral RNA-dependent RNA polymerases also create aberrant products with large internal deletions, referred to as defective viral genomes (DVGs) or sometimes deletions-containing viral genomes (DelVGs). DVGs can be incorporated into new virions, forming defective interfering particles (DIPs) that directly inhibit replication of the wild-type (WT) virus. Despite almost 70 years studying influenza virus DIPs, the exact process by which they interfere with WT virus is still not known. It has been hypothesized that the DVG RNA itself acts as a parasite to consume resources at the expense of full-length genomes (e.g., monopolize the viral polymerase, use available substrate, etc.).

### SUMMARY

The disclosure provides for methods for making and using modified influenza gene products, alone or in combination, e.g., to inhibit wild-type influenza virus replication, to serve as an immunostimulatory agent, and/or as attenuated vaccine backbones. In one embodiment, the genomes of the DIPs provide for inhibitory activity, producing a dual effect in which both the RNA itself and the encoded protein coordinate to interfere with replication. Thus, the ability of DIPs to block replication of WT virus provides for a treatment for infection, use as an immunostimulatory agent, and as attenuated viruses for vaccination.

In particular, defective viral genomes (DVGs) are not simply inhibitory RNAs, but also have the potential to code for cryptic viral proteins that may be truncated, mutated, or frame-shifted that maintain protein coding potential. DVGs were identified in all eight viral segments. These proteins may be produced during viral infection. More importantly, these proteins may alter viral replication. In one embodiment, multiple DVG-encoded proteins were identified that function as dominant-negative inhibitors of the viral polymerase by blocking polymerase activity and decreasing viral replication. Viruses were produced that express these proteins in the place of the normal wild-type gene product. Mechanistic assays showed that these proteins compete with and disrupt assembly of the viral polymerase. Because these proteins interfere with a conserved step in viral polymerase assembly, they are likely to be broadly inhibitory across diverse influenza isolates.

The disclosure provides a recombinant influenza virus, e.g., that may express vRNAs that are inhibitory and/or express mutant viral proteins that are inhibitory, which may be useful to prevent, inhibit or treat an influenza virus infection and/or as a vaccine, which virus optionally may stably express a foreign gene and so may provide for multivalency.

In one embodiment, the invention provides isolated influenza virus that has a viral gene segment that does not comprise contiguous nucleic acid sequences corresponding to those encoding PB2 (a mutant PB2 viral gene segment), a protein which is one of the viral RNA polymerase subunits and is essential for virus replication. To prepare such a virus in cell culture, a cell line may be employed that expresses PB2 in trans in combination with vectors for influenza virus vRNA production, but not one for a wild-type PB2 viral gene segment, and in one embodiment, vectors for influenza virus mRNA protein production. The resulting virus is not competent to express PB2 after infection of cells that do not express PB2 in trans or are not infected with helper virus. However, virions produced from cells that express PB2 in trans contain PB2. Such an infectious influenza virus with a mutant PB2 viral gene segment may be generated in multiple cell lines that express PB2 in trans, such as PB2-expressing 293 human embryonic kidney (293), human lung adenocarcinoma epithelial (A549), or 2,6-linked sialyltransferase-overexpressing Madin-Darby canine kidney (MDCK) cells (AX4 cells), resulting in high virus titers of, for example, at least 10⁴, 10⁵, 10⁶, 10⁷ or 10⁸ PFU/mL, or more.

In one embodiment, the disclosure provides an isolated recombinant influenza virus comprising 8 gene segments including a PA viral gene segment, a PB1 viral gene segment, a mutant PB2 viral gene segment, a HA viral gene segment, a NA viral gene segment, a NP viral gene segment, a M (M1 and M2) viral gene segment, and a NS (NS1 and NS2) viral gene segment. In another embodiment, the invention provides an isolated recombinant influenza virus comprising 8 gene segments including a PA viral gene segment, a PB1 viral gene segment, a mutant PB2 viral gene segment, a HA viral gene segment, a NA (NA and NB) viral gene segment, a NP viral gene segment, a M (M1 and BM2) viral gene segment, and a NS (NS1 and NS2) viral gene segment. In one embodiment, the recombinant influenza virus has a M viral gene segment for M1 and M2. In one embodiment, the recombinant influenza virus has a NA viral gene segment for NB and NA. In one embodiment, the mutant PB2 viral gene segment includes 5′ and/or 3′ PB2 viral non-coding and coding incorporation (packaging) sequences, optionally flanking a heterologous nucleotide sequence, and does not include contiguous sequences corresponding to sequences encoding a functional PB2. The PB2 open reading frame in the mutant PB2 viral gene segment may be replaced with or disrupted by a heterologous nucleotide sequence, e.g., with a gene encoding an antigen from a pathogen. In one embodiment, the PB2 coding region in the mutant PB2 viral gene segment may include mutations such as insertions or deletions of one or more nucleotides or those that result in one or more amino acid substitutions or a stop codon, or any combination thereof, that yields a non-functional PB2 coding sequence. In one embodiment, the heterologous nucleotide sequence is about 30 to about 5,000, e.g., about 100 to about 3,500 or about 500 to about 2,500, nucleotides in length. In one embodiment, DVGs are from 200-500 nucleotides in length.

The viruses may thus be used as an influenza vaccine, e.g., a prophylactic, as an adjuvant, e.g., one that enhances immune stimulation, optionally used with another immunogen, or as a therapeutic to decrease virus replication in vivo optionally in conjunction with traditional influenza virus vaccines, e.g., those having live, attenuated or killed viruses. The viruses of the disclosure may elicit a better immune response than wild-type viruses. The virus-like particles (VLPs) (which may be consider knock-out (KO) viruses) of the disclosure contain RNA, which is an adjuvant that enhances the host's immune response against the virus.

In one embodiment, the mutant PB2 viral gene segment has a deletion of PB2 coding sequences that results in a cryptic protein or a deletion of PB2 coding sequences and an insertion of heterologous nucleotide sequences. That virus replicates in vitro when PB2 is supplied in trans to titers that may be substantially the same or at most 10, 100 or 1,000 fold less than a corresponding wild-type influenza virus, but is attenuated in vivo or in vitro in the absence of PB2 supplied in trans. In one embodiment, the deletion of PB2 coding sequences includes 400, 500, 600, 700, 800, 900, 1000, 1200, or more contiguous or noncontiguous nucleotides of PB2 coding sequence, which may result in a mutant PB2 gene segment of from about 200 to 400, 400 to 600, 600 to 800 or 800 to 1000 nucleotides in length. In one embodiment, the deletion includes at least 10%, 30%, 40%, 50%, 60%, 70%, 80% or 90%, or a percent numerical value that is any integer between 10 and 70, but not all, of the PB2 coding region. In one embodiment, the deletion of PB2 coding sequences does not include the deletion of 5′ or 3′ coding sequences that enhance incorporation of the resulting viral gene segment into virions, e.g., sequences that are contiguous to 3′ or 5′ non-coding and coding PB2 sequences, relative to a recombinant viral gene segment with only non-coding PB2 incorporation sequences.

In one embodiment, the deletion in the PB2 gene segment results in a frame-shift. In one embodiment, the deletion in the PB2 gene segment does not result in a frame-shift.

In one embodiment of the disclosure, the heterologous nucleotide sequence may encode a heterologous protein (a non-influenza viral protein such as a glycoprotein or a cytosolic, nuclear or mitochondrial specific protein, or any antigenic protein such as an antigen from a microbial pathogen), which may confer a detectable phenotype. In one embodiment, the heterologous nucleotide sequence may be fused to truncated portions of PB2 coding sequences, e.g., those corresponding to 5′ or 3′ PB2 coding incorporation sequences, optionally forming a chimeric protein. In one embodiment, the heterologous nucleotide sequence replaces or is introduced to sequences in the viral gene segment corresponding to the coding region for that segment, so as not to disrupt the incorporation sequences in the coding region of the gene segment. For instance, the heterologous nucleotide sequence may be flanked by about 3 to about 400 nucleotides of the 5′ and/or 3′ PB2 coding region adjacent to non-coding sequence. In one embodiment, the 3′ PB2 incorporation sequences correspond to nucleotides 3 to 400, nucleotides 3 to 300, nucleotides 3 to 110, nucleotides 3 to 106, or any integer between 3 and 400, of the N-terminal and/or C-terminal PB2 coding region.

A vector for vRNA production of the mutant PB2 gene segment is introduced into a cell along with a vector or vectors for vRNA production for PA vRNA, PB1 vRNA, NP vRNA, HA vRNA, NA vRNA, M vRNA, and NS (NS1 and/or NS2) vRNA, and vectors for mRNA (protein) production for one or more of PA, PB1, PB2, and NP, or vectors for mRNA production of up to three of PA, PB1, PB2, and NP, where the cell stably expresses the remaining viral protein(s), and optionally expresses HA, NA, M, e.g., M1 and M2, NS1 and/or NS2.

Thus, the disclosure provides a vaccine or immunogenic composition comprising a virus having the mutant PB2 viral segment, VLP having the mutant PB2 viral segment, isolated vector comprising sequences for the mutant PB2 viral segment or a protein, e.g., encoded by the mutant PB2 viral segments disclosed herein, and a method of using the vaccine or immunogenic composition, e.g., to immunize a vertebrate, e.g., an avian or a mammal, such as a human, or induce or enhance an immune response in a vertebrate. In one embodiment, the composition or vaccine is formulated for intranasal administration. In one embodiment, the recombinant virus in a vaccine comprises a HA gene segment for influenza A virus HA, e.g., H1, H2, H3, H5, H7, or H9 HA. In one embodiment, the HA in the recombinant virus in a vaccine is modified at the HA cleavage site. In one embodiment, the vaccine comprises at least one influenza virus strain that is different than the recombinant virus of the disclosure, for instance, the vaccine comprises two or three different influenza viruses.

The invention provides a plurality of vectors to prepare influenza A virus having one or more vectors which include transcription cassettes for vRNA production and transcription cassettes for mRNA production. The transcription cassettes for vRNA production are a transcription cassette comprising a promoter for vRNA production, e.g., a PolI promoter, operably linked to an influenza virus PA DNA in an orientation for genomic viral RNA production linked to a transcription termination sequence that results in influenza virus-like vRNA termini, for instance, a PolI transcription termination sequence, a transcription cassette comprising a promoter for vRNA production, e.g., a PolI promoter, operably linked to an influenza virus PB1 DNA in an orientation for genomic viral RNA production linked to a transcription termination sequence that results in influenza virus-like vRNA termini, for instance, PolI transcription termination sequence, a transcription cassette comprising a promoter for vRNA production, e.g., a PolI promoter, operably linked to a mutant influenza virus PB2 DNA in an orientation for genomic viral RNA production linked to a transcription termination sequence that results in influenza virus-like vRNA termini, for instance, a PolI transcription termination sequence, a transcription cassette comprising a promoter for vRNA production, e.g., a PolI promoter, operably linked to an influenza virus HA DNA in an orientation for genomic viral RNA production linked to a transcription termination sequence that results in influenza virus-like vRNA termini, for instance, a PolI transcription termination sequence, a transcription cassette comprising a promoter for vRNA production, e.g., a PolI promoter, operably linked to an influenza virus NA DNA in an orientation for genomic viral RNA production linked to a transcription termination sequence that results in influenza virus-like vRNA termini, for instance, a PolI transcription termination sequence, a transcription cassette comprising a promoter for vRNA production, e.g., a PolI promoter, operably linked to an influenza virus NP DNA in an orientation for genomic viral RNA production linked to a transcription termination sequence that results in influenza virus-like vRNA termini, for instance, a PolI transcription termination sequence, a transcription cassette comprising a promoter for vRNA production, e.g., a PolI promoter, operably linked to an influenza virus M DNA in an orientation for genomic viral RNA production linked to a transcription termination sequence that results in influenza virus-like vRNA termini, for instance, a PolI transcription termination sequence, and a transcription cassette comprising a promoter for vRNA production, e.g., a PolI promoter, operably linked to an influenza virus NS (NS1 and NS2) DNA in an orientation for genomic viral RNA production linked to a transcription termination sequence that results in influenza virus-like vRNA termini, for instance, a PolI transcription termination sequence. The mutant PB2 viral gene segment includes 5′ and 3′ incorporation sequences including 3′ or 5′ coding and non-coding incorporation sequences flanking a nucleotide sequence having PB2 sequences that encode an inhibitory protein or a nucleotide sequence having PB2 sequences that encode a polypeptide having at least the 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39 N-terminal amino acids of PB2, wherein the mutant PB2 segment does not include contiguous sequences corresponding to sequences encoding a functional PB2. The transcription cassettes for mRNA production are a transcription cassette comprising a PolII promoter operably linked to a DNA coding region for influenza virus PA linked to a PolII transcription termination sequence, a transcription cassette comprising a PolII promoter operably linked to a DNA coding region for influenza virus PB1 linked to a PolII transcription termination sequence, and a transcription cassette comprising a PolII promoter operably linked to a DNA coding region for influenza virus NP linked to a PolII transcription termination sequence, and optionally a transcription cassette comprising a PolII promoter operably linked to a DNA coding region for influenza virus one or more of PB2, HA, NA, NS1, NS2, M1 and/or M2 linked to a PolII transcription termination sequence. Further provided is a composition having the vectors, and a method which employs the vectors.

The disclosure also provides a plurality of vectors to prepare an 8 segment influenza B virus having one or more vectors which include transcription cassettes for vRNA production and transcription cassettes for mRNA production. The transcription cassettes for vRNA production are a transcription cassette comprising a promoter for vRNA production, e.g., a PolI promoter, operably linked to an influenza virus PA DNA in an orientation for genomic viral RNA production linked to a transcription termination sequence that results in influenza virus-like vRNA termini, for instance, a PolI transcription termination sequence, a transcription cassette comprising a promoter for vRNA production, e.g., a PolI promoter, operably linked to an influenza virus PB1 DNA in an orientation for genomic viral RNA production linked to a transcription termination sequence that results in influenza virus-like vRNA termini, for instance, a PolI transcription termination sequence, a transcription cassette comprising a promoter for vRNA production, e.g., a PolI promoter, operably linked to a mutant influenza virus PB2 DNA in an orientation for genomic viral RNA production linked to a transcription termination sequence that results in influenza virus-like vRNA termini, for instance, a PolI transcription termination sequence, a transcription cassette comprising a promoter for vRNA production, e.g., a PolI promoter, operably linked to an influenza virus HA DNA in an orientation for genomic viral RNA production linked to a transcription termination sequence that results in influenza virus-like vRNA termini, for instance, a PolI transcription termination sequence, a transcription cassette comprising a promoter for vRNA production, e.g., a PolI promoter, operably linked to an influenza virus NA and NB DNA in an orientation for genomic viral RNA production linked to a transcription termination sequence that results in influenza virus-like vRNA termini, for instance, a PolI transcription termination sequence, a transcription cassette comprising a promoter for vRNA production, e.g., a PolI promoter, operably linked to an influenza virus NP DNA in an orientation for genomic viral RNA production linked to a transcription termination sequence that results in influenza virus-like vRNA termini, for instance, a PolI transcription termination sequence, a transcription cassette comprising a promoter for vRNA production, e.g., a PolI promoter, operably linked to an influenza virus M DNA in an orientation for genomic viral RNA production linked to a transcription termination sequence that results in influenza virus-like vRNA termini, for instance, a PolI transcription termination sequence, and a transcription cassette comprising a PolI promoter operably linked to an influenza virus NS (NS1 and NS2) DNA in an orientation for genomic viral RNA production linked to a PolI transcription termination sequence. The mutant PB2 viral gene segment includes 5′ and 3′ incorporation sequences including 3′ or 5′ coding and non-coding incorporation sequences flanking a nucleotide sequence having PB2 sequences that encode an inhibitory protein or a nucleotide sequence having PB2 sequences that encode a polypeptide having at least the 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39 N-terminal amino acids of PB2, wherein the mutant PB2 segment does not include contiguous sequences corresponding to sequences encoding a functional PB2. The transcription cassettes for mRNA production are a transcription cassette comprising a PolII promoter operably linked to a DNA coding region for influenza virus PA linked to a PolII transcription termination sequence, a transcription cassette comprising a PolII promoter operably linked to a DNA coding region for influenza virus PB1 linked to a PolII transcription termination sequence, and a transcription cassette comprising a PolII promoter operably linked to a DNA coding region for influenza virus NP linked to a PolII transcription termination sequence, and optionally a transcription cassette comprising a PolII promoter operably linked to a DNA coding region for influenza virus one or more of PB2, HA, NA, NS1, NS2, M1 and/or BM2 linked to a PolII transcription termination sequence. Further provided is a composition having the vectors and a method which employs the vectors.

In one embodiment, the promoter in a vRNA vector includes but is not limited to a RNA polymerase I (PolI) promoter, e.g., a human RNA PolI promoter, a RNA polymerase II (PolII) promoter, a RNA polymerase III promoter, a SP6 promoter, a T7 promoter, or a T3 promoter. In one embodiment, one or more vRNA vectors include a PolII promoter and ribozyme sequences 5′ to influenza virus sequences and the same or different ribozyme sequences 3′ to the influenza virus sequences. In one embodiment, the mutant PB2 gene segment is in a vector and is operably linked to a promoter including, but not limited to, a RNA PolI promoter, e.g., a human RNA PolI promoter, a RNA PolII promoter, a RNA polymerase III promoter, a SP6 promoter, a T7 promoter, or a T3 promoter. In one embodiment, the vRNA vectors include a transcription termination sequence including, but not limited to, a PolI transcription termination sequence, a PolII transcription termination sequence, or a PolIII transcription termination sequence, or one or more ribozymes.

A plurality of the vectors may be physically linked or each vector may be present on an individual plasmid or other, e.g., linear, nucleic acid delivery vehicle. In one embodiment, each vRNA production vector is on a separate plasmid. In one embodiment, each mRNA production vector is on a separate plasmid. In one embodiment, one or more vectors for vRNA production are on the same plasmid (see, e.g., U.S. published application No. 20060166321, the disclosure of which is incorporated by reference herein). In one embodiment, one or more vectors for mRNA production are on the same plasmid (see, e.g., U.S. published application No. 2006/0166321). In one embodiment, the vRNA vectors employed in the method are on one plasmid or on two or three different plasmids. In one embodiment, the mRNA vectors for PA, PB1, and NP, and optionally PB2, employed in the method are on one plasmid or on two or three different plasmids.

Also provided is a host cell comprising a vector expressing PB2, e.g., PB2 from PR8 or other master vaccine strain. In one embodiment, the PB2 has at least 90%, 95%, 98%, 99% or 100% identity to PB2 encoded by SEQ ID NO:50. In one embodiment, the host cell is transduced with a viral vector, e.g., a vector which is stably maintained in the cell as an episome or integrated into a chromosome, such as a lentiviral or retroviral vector. In one embodiment, the host cell further includes one or more vectors which include transcription cassettes for transient vRNA production and transcription cassettes for transient mRNA production. The transcription cassettes for vRNA production are a transcription cassette comprising a promoter for vRNA production, e.g., a PolI promoter, operably linked to an influenza virus PA DNA in an orientation for genomic viral RNA production linked to a transcription termination sequence that results in influenza virus-like vRNA termini, for instance, a PolI transcription termination sequence, a transcription cassette comprising a promoter for vRNA production, e.g., a PolI promoter operably linked to an influenza virus PB1 DNA in an orientation for genomic viral RNA production linked to a transcription termination sequence that results in influenza virus-like vRNA termini, for instance, a PolI transcription termination sequence, a transcription cassette comprising a promoter for vRNA production, e.g., a PolI promoter, operably linked to a mutant influenza virus PB2 DNA in an orientation for genomic viral RNA production linked to a transcription termination sequence that results in influenza virus-like vRNA termini, for instance, a PolI transcription termination sequence, a transcription cassette comprising a promoter for vRNA production, e.g., a PolI promoter, operably linked to an influenza virus HA DNA in an orientation for genomic viral RNA production linked to a transcription termination sequence that results in influenza virus-like vRNA termini, for instance, a PolI transcription termination sequence, a transcription cassette comprising a promoter for vRNA production, e.g., a PolI promoter, operably linked to an influenza virus NA DNA in an orientation for genomic viral RNA production linked to a transcription termination sequence that results in influenza virus-like vRNA termini, for instance, a PolI transcription termination sequence, a transcription cassette comprising a promoter for vRNA production, e.g., a PolI promoter, operably linked to an influenza virus NP DNA in an orientation for genomic viral RNA production linked to a transcription termination sequence that results in influenza virus-like vRNA termini, for instance, a PolI transcription termination sequence, a transcription cassette comprising a promoter for vRNA production, e.g., a PolI promoter, operably linked to an influenza virus M DNA in an orientation for genomic viral RNA production linked to a transcription termination sequence that results in influenza virus-like vRNA termini, for instance, a PolI transcription termination sequence, and a transcription cassette comprising a promoter for vRNA production, e.g., a PolI promoter, operably linked to an influenza virus NS (NS1 and NS2) DNA in an orientation for genomic viral RNA production linked to a transcription termination sequence that results in influenza virus-like vRNA termini, for instance, a PolI transcription termination sequence. The mutant PB2 viral gene segment includes 5′ and 3′ incorporation sequences including 3′ or 5′ coding and non-coding incorporation sequences flanking a nucleotide sequence having PB2 sequences that encode an inhibitory protein or a nucleotide sequence having PB2 sequences that encode a polypeptide having at least the 39 N-terminal amino acids of PB2, wherein the mutant PB2 segment does not include contiguous sequences corresponding to sequences encoding a functional PB2 The transcription cassettes for mRNA production are a transcription cassette comprising a PolII promoter operably linked to a DNA coding region for influenza virus PA linked to a PolII transcription termination sequence, a transcription cassette comprising a PolII promoter operably linked to a DNA coding region for influenza virus PB1 linked to a PolII transcription termination sequence, and a transcription cassette comprising a PolII promoter operably linked to a DNA coding region for influenza virus NP linked to a PolII transcription termination sequence. The host cell does not include sequences corresponding to PB2 coding sequences for vRNA production of a wild-type PB2 viral gene segment.

The disclosure also provides a method to prepare influenza virus, e.g., using a host cell of the disclosure. The method comprises contacting a cell with a plurality of the vectors of the disclosure, e.g., sequentially or simultaneously, in an amount effective to yield infectious influenza virus. The invention also includes isolating virus from a cell contacted with the plurality of vectors. Thus, the invention further provides isolated virus, as well as a host cell contacted with virus of the disclosure. In another embodiment, the disclosure includes contacting the cell with one or more vectors, either vRNA or protein production vectors, prior to other vectors, either vRNA or protein production vectors.

In one embodiment, the disclosure provides a method of preparing a recombinant influenza virus comprising a mutant PB2 viral gene segment. The method comprises contacting a host cell with a plurality of influenza vectors, including a vector comprising the mutant PB2 gene segment sequence, so as to yield recombinant virus. For example, the host cell is contacted with vectors for vRNA production including a vector comprising a promoter for vRNA production operably linked to an influenza virus PA DNA linked to a transcription termination sequence, a vector comprising a promoter for vRNA production operably linked to an influenza virus PB1 DNA linked to a transcription termination sequence, a vector comprising a promoter for vRNA production operably linked to a mutant influenza virus PB2 DNA linked to a transcription termination sequence, a vector comprising a promoter for vRNA production operably linked to an influenza virus HA DNA linked to a transcription termination sequence, a vector comprising a promoter for vRNA production operably linked to an influenza virus NP DNA linked to a transcription termination sequence, a vector comprising a promoter for vRNA production operably linked to an influenza virus NA DNA linked to a transcription termination sequence, a vector comprising a promoter for vRNA production operably linked to an influenza virus M DNA linked to a transcription termination sequence, and a vector comprising a promoter for vRNA production operably linked to an influenza virus NS (NS1 and NS2) DNA linked to a transcription termination sequence, wherein the mutant PB2 viral gene segment includes 5′ and 3′ incorporation sequences including 3′ or 5′ coding and non-coding incorporation sequences flanking a nucleotide sequence having PB2 sequences that encode an inhibitory protein or a nucleotide sequence having PB2 sequences that encode a polypeptide having at least the 39 N-terminal amino acids of PB2, wherein the mutant PB2 segment does not include contiguous sequences corresponding to sequences encoding a functional PB2, and vectors for mRNA production including a vector comprising a promoter operably linked to a DNA segment encoding influenza virus PA, a vector comprising a promoter operably linked to a DNA segment encoding influenza virus PB1, a vector comprising a promoter operably linked to a DNA segment encoding influenza virus NP, wherein the cell is not contacted with sequences corresponding to PB2 coding sequences for vRNA production. Optionally, the host cell is contacted with a vector comprising a promoter operably linked to a DNA segment encoding influenza virus HA, a vector comprising a promoter operably linked to a DNA segment encoding influenza virus NA, a vector comprising a promoter operably linked to a DNA segment encoding influenza virus M1, a vector comprising a promoter operably linked to a DNA segment encoding a M2 protein, e.g., a mutant M2 protein, and a vector comprising a promoter operably linked to a DNA segment encoding influenza virus NS1 and/or NS2. In one embodiment, separate vectors for M1 and M2 mRNA, and/or for NS1 and NS2 mRNA are provided and employed.

In one embodiment of a method of preparing an influenza virus of the disclosure, each transcription cassette is on a plasmid vector. In one embodiment of a method of preparing the influenza virus of the disclosure, one or more transcription cassettes are on one or more plasmid vectors, e.g., one plasmid vector has transcription cassettes for vRNA production of PA, PB1, HA, NP, NA, M1, NS1 and/or NS2, and the mutant PB2 cDNAs. In one embodiment of a method of preparing the virus of the disclosure, one plasmid vector has one of the transcription cassette for mRNA production and another plasmid vector has the other transcription cassettes for mRNA production. In one embodiment of a method of preparing the influenza virus of the disclosure, three plasmid vectors for mRNA production are employed, each with one of the transcription cassettes for mRNA production. In one embodiment of a method of preparing an influenza virus of the disclosure, one plasmid vector has six of the transcription cassettes for vRNA production and another plasmid vector has the other transcription cassette for vRNA production, e.g., one plasmid vector has one of the transcription cassettes for mRNA production and another plasmid vector has the other transcription cassettes for mRNA production. In one embodiment of a method of preparing an influenza virus of the disclosure, three plasmid vectors for mRNA production are employed. In one embodiment of a method of preparing an influenza virus of the disclosure, one plasmid has the three transcription cassettes for mRNA production. In one embodiment of a method of preparing an influenza virus of the disclosure, the HA cDNA encodes an avirulent cleavage site. In one embodiment of a method of preparing an influenza virus of the disclosure, the HA and NA are from the same virus isolate. In one embodiment of a method of preparing an influenza virus of the disclosure, the HA is a type B HA.

The promoter or transcription termination sequence in a vRNA or virus protein expression vector may be the same or different relative to the promoter or any other vector. In one embodiment, the vector or plasmid which expresses influenza vRNA comprises a promoter suitable for expression in at least one particular host cell, e.g., avian or mammalian host cells such as canine, feline, equine, bovine, ovine, or primate cells including human cells, or for expression in more than one host. In one embodiment, the PolI promoter for each PolI containing vector is the same. In one embodiment, the PolI promoter is a human PolI promoter. In one embodiment, the PolII promoter for each PolII containing vector is the same. In one embodiment, the PolII promoter for two or more, but not all, of the PolII containing vectors, is the same. In one embodiment, the PolII promoter for each PolII containing vector is different.

In another embodiment, the method includes contacting a host cell with a vector having a PolII promoter linked to a PolI transcription termination sequence linked to an influenza virus PA DNA linked to a PolI promoter linked to a PolII transcription termination sequence (a bidirectional cassette), a vector having a PolII promoter linked to a PolI transcription termination sequence linked to an influenza virus PB1 DNA linked to a PolI promoter linked to a PolII transcription termination sequence, a vector having a PolII promoter linked to a PolI transcription termination sequence linked to a mutant influenza virus PB2 DNA linked to a PolI promoter linked to a PolII transcription terminator sequence, a vector having a PolII promoter linked to a PolI transcription termination sequence linked to an influenza virus HA DNA linked to a PolI promoter linked to a PolII transcription termination sequence, a vector having a PolII promoter linked to a PolI transcription termination sequence linked to an influenza virus NP DNA linked to a PolI promoter linked to a PolII transcription termination sequence, a vector having a PolII promoter linked to a PolI transcription termination sequence linked to an influenza virus NA DNA linked to a PolI promoter linked to a PolII transcription termination sequence, a vector having a PolII promoter linked to a PolI transcription termination sequence linked to an influenza virus M DNA linked to a PolI promoter linked to PolII transcription termination sequence, and a vector having a PolII promoter linked to a PolI transcription termination sequence linked to an influenza virus NS1 and/or NS2 DNA linked to a PolI promoter linked to PolII transcription termination sequence. The host cell comprises PB2 DNA expressing a PB2 protein, e.g., from a chicken beta-actin promoter. No sources of vRNA for wild-type PB2 are present so that replication-incompetent virus is provided.

In one embodiment, the promoter for vRNA production in a bidirectional cassette includes but is not limited to a RNA polymerase I (PolI) promoter, e.g., a human RNA PolI promoter, a RNA polymerase II (PolII) promoter, a RNA polymerase III promoter, a SP6 promoter, a T7 promoter, or a T3 promoter. In one embodiment, one or more vRNA vectors include a PolII promoter and ribozyme sequences 5′ to influenza virus sequences and the same or different ribozyme sequences 3′ to the influenza virus sequences. In one embodiment, the mutant PB2 gene segment is in a vector and is operably linked to a promoter including, but not limited to, a RNA PolI promoter, e.g., a human RNA PolI promoter, a RNA PolII promoter, a RNA polymerase III promoter, a SP6 promoter, a T7 promoter, or a T3 promoter. In one embodiment, the vRNA vectors include a transcription termination sequence including, but not limited to, a PolI transcription termination sequence, a PolII transcription termination sequence, or a PolIII transcription termination sequence, or one or more ribozymes. Ribozymes within the scope of the disclosure include, but are not limited to, tetrahymena ribozymes, RNase P, hammerhead ribozymes, hairpin ribozymes, hepatitis ribozyme, as well as synthetic ribozymes. In one embodiment, at least one vector for vRNA comprises a RNA polymerase II promoter linked to a ribozyme sequence linked to viral coding sequences linked to another ribozyme sequences, optionally linked to a RNA polymerase II transcription termination sequence. In one embodiment, at least 2, e.g., 3, 4, 5, 6, 7 or 8, vectors for vRNA production comprise a RNA polymerase II promoter, a first ribozyme sequence, which is 5′ to a sequence corresponding to viral sequences including viral coding sequences, which is 5′ to a second ribozyme sequence, which is 5′ to a transcription termination sequence. Each RNA polymerase II promoter in each vRNA vector may be the same or different as the RNA polymerase II promoter in any other vRNA vector. Similarly, each ribozyme sequence in each vRNA vector may be the same or different as the ribozyme sequences in any other vRNA vector. In one embodiment, the ribozyme sequences in a single vector are not the same.

The plurality of vectors, compositions and host cells of the disclosure may also include another vector for vRNA production or protein production that includes heterologous sequences, e.g., for a therapeutic or prophylactic gene of interest e.g., an immunogen for a cancer associated antigen or for a pathogen such as a bacteria, a noninfluenza virus, fungus, or other pathogen. For example, the vector or plasmid comprising the gene or cDNA of interest may substitute for a vector or plasmid for an influenza viral gene or may be in addition to vectors or plasmids for all influenza viral genes. Thus, another embodiment of the disclosure comprises a composition or plurality of vectors as described above in which one of the vectors is replaced with, or further comprises, S′ influenza virus sequences optionally including 5′ influenza virus coding sequences or a portion thereof, linked to a desired nucleic acid sequence, e.g., a desired cDNA, linked to 3′ influenza virus sequences optionally including 3′ influenza virus coding sequences or a portion thereof. In one embodiment, the desired nucleic acid sequence such as a cDNA is in an antisense (antigenomic) orientation. The introduction of such a vector in conjunction with the other vectors described above to a host cell permissive for influenza virus replication results in recombinant virus comprising vRNA corresponding to the heterologous sequences of the vector.

The DNA for vRNA production of NA may be from any NA, e.g., any of N1-N11, a chimeric NA sequence or any non-native NA sequence, and the DNA for vRNA production of HA may be from any HA, e.g., H1-H18, a chimeric HA sequence or any non-native HA sequence. In one embodiment, other attenuating mutations may be introduced to the vectors, e.g., a mutation in a HA cleavage site that results in a site that is not cleaved. The DNAs for vRNA production of NA and HA may be from different strains or isolates relative to those for the (6:1:1 reassortants) or from the same strain or isolate (6:2 reassortants), the NA may be from the same strain or isolate as that for the internal genes (7:1 reassortant), or one of the internal genes, NA and HA may be from the same strain or isolate (5:3 reassortant).

Viruses that may provide the internal genes for reassortants within the scope of the disclosure include viruses that have high titers in Vero cells, e.g., titers of at least about 10⁵ PFU/mL, e.g., at least 10⁶ PFU/mL, 10⁷ PFU/mL or 10⁸ PFU/mL; high titers in embryonated eggs, e.g., titers of at least about 10⁷ EID₅₀/mL, e.g., at least 10⁸ EID₅₀/mL, 10⁹ EID₅₀/mL or 10¹⁰ EID₅₀/mL; high titers in MDCK, e.g., AX5, cells, e.g., titers of at least about 10⁶ PFU/mL, 10⁷ PFU/mL, e.g., at least 10⁸ PFU/mL, or high titers in two of more of those host cells. In one embodiment, the DNAs for vRNA production of PB1 vRNA, mutant PB2 vRNA, PA vRNA, NP vRNA, M vRNA (for M1 and/or M2 or M1 and/or BM2), and/or NS vRNA (for NS1 and/or NS2), may have sequences from an influenza virus that replicates to high titers in cultured mammalian cells such as AX4 cells, Vero cells or PER.C6® cells and also optionally embryonated eggs, and/or from a vaccine virus, e.g., one that does not cause significant disease in humans.

In one embodiment, the DNAs for the internal genes for PB1, PB2, PA, NP, M, and NS encode proteins with substantially the same activity as a corresponding polypeptide encoded by one of SEQ ID NOs:50-52, 54 or 56-57. As used herein, “substantially the same activity” includes an activity that is about 0.1%, 1%, 10%, 30%, 50%, 90%, e.g., up to 100% or more, or detectable protein level that is about 80%, 90% or more, the activity or protein level, respectively, of the corresponding full-length polypeptide. In one embodiment, the nucleic acid a sequence encoding a polypeptide which is substantially the same as, e.g., having at least 80%, e.g., 90%, 92%, 95%, 97% or 99%, including any integer between 80 and 99, contiguous amino acid sequence identity to, a polypeptide encoded by one of SEQ ID NOs: 50-52, 54 or 56-57. In one embodiment, the isolated and/or purified nucleic acid molecule comprises a nucleotide sequence which is substantially the same as, e.g., having at least 50%, e.g., 60%, 70%, 80% or 90%, including any integer between 50 and 100, or more contiguous nucleic acid sequence identity to one of SEQ ID NOs: 1-6 or 33-38 and, in one embodiment, also encodes a polypeptide having at least 80%, e.g., 90%, 92%, 95%, 97% or 99%, including any integer between 80 and 99, contiguous amino acid sequence identity to a polypeptide encoded by one of SEQ ID NOs: 50-52, 54 or 56-57. In one embodiment, the influenza virus polypeptide has one or more, for instance, 2, 5, 10, 15, 20 or more, conservative amino acids substitutions, e.g., conservative substitutions of up to 10% or 20% of the residues, relative to a polypeptide encoded by one of SEQ ID NOs: 50-52, 54 or 56-57. Conservative amino acid substitutions refer to the interchangeability of residues having similar side chains. For example, a group of amino acids having aliphatic side chains is glycine, alanine, valine, leucine, and isoleucine, a group of amino acids having aliphatic-hydroxyl side chains is serine and threonine; a group of amino acids having amide-containing side chains is asparagine and glutamine; a group of amino acids having aromatic side chains is phenylalanine, tyrosine and tryptophan; a group of amino acids having basic side chains is lysine, arginine and histidine; and a group of amino acids having sulfur-containing side chain is cysteine and methionine. In one embodiment, conservative amino acid substitution groups are: valine-leucine-isoleucine, phenylalanine-tyrosine; lysine-arginine; alanine-valine; glutamic-aspartic; and asparagine-glutamine. In one embodiment, the influenza virus polypeptide has one or more, for instance, 2, 3 or 4, nonconservative amino acid substitutions, relative to a polypeptide encoded by one of SEQ ID NOs: 50-52, 54 or 56-57.

The methods of producing virus described herein, which do not require helper virus infection, are useful in viral mutagenesis studies, and in the production of vaccines (e.g., for AIDS, influenza, hepatitis B, hepatitis C, rhinovirus, filoviruses, malaria, herpes, and foot and mouth disease) and gene therapy vectors (e.g., for cancer, AIDS, adenosine deaminase, muscular dystrophy, ornithine transcarbamylase deficiency and central nervous system tumors). Thus, a virus for use in medical therapy (e.g., for a vaccine or gene therapy) is provided.

The methods include administering to a host organism, e.g., a mammal, an effective amount of the influenza virus, VLP, isolated nucleic acid or isolated protein of the disclosure, e.g., a live or inactivated virus preparation, optionally in combination with an adjuvant and/or a carrier, e.g., in an amount effective to prevent or ameliorate infection of an animal such as a mammal by that virus or an antigenically closely related virus. In one embodiment, the virus is administered intramuscularly while in another embodiment, the virus is administered intranasally. In some dosing protocols, all doses may be administered intramuscularly or intranasally, while in others a combination of intramuscular and intranasal administration is employed. In one embodiment, two to three doses are administered. The vaccine may be multivalent as a result of the heterologous nucleotide sequence introduced into a viral gene segment in the influenza virus of the disclosure. The vaccine may further contain other isolates of influenza virus including recombinant influenza virus, other pathogen(s), additional biological agents or microbial components, e.g., to form a multivalent vaccine. In one embodiment, intranasal vaccination, for instance containing with inactivated influenza virus, and a mucosal adjuvant may induce virus-specific IgA and neutralizing antibody in the nasopharynx as well as serum IgG.

The compositions of the disclosure may employed with other anti-virals, e.g., amantadine, rimantadine, and/or neuraminidase inhibitors, e.g., may be administered separately in conjunction with those anti-virals, for instance, administered before, during and/or after.

In one embodiment, the compositions of the disclosure may be vaccine vectors for influenza virus and for at least one other pathogen, such as a viral or bacterial pathogen, or for a pathogen other than influenza virus, pathogens including but not limited to, lentiviruses such as HIV, hepatitis B virus, hepatitis C virus, herpes viruses such as CMV or HSV, Foot and Mouth Disease Virus, Measles virus, Rubella virus, Mumps virus, human Rhinovirus, Parainfluenza viruses, such as respiratory syncytial virus and human parainfluenza virus type 1, Coronavirus, Nipah virus, Hantavirus, Japanese encephalitis virus, Rotavirus, Dengue virus, West Nile virus, Streptococcus pneumoniae, Mycobacterium tuberculosis, Bordetella pertussis, or Haemophilus influenza. For example, the influenza virus of the disclosure may include sequences for H protein of Measles virus, viral envelope protein E1 of Rubella virus, HN protein of Mumps virus, RV capsid protein VP1 of human Rhinovirus, G protein of Respiratory syncytial virus, S protein of Coronavirus, G or F protein of Nipah virus, G protein of Hantavirus, E protein of Japanese encephalitis virus, VP6 of Rotavirus, E protein of Dengue virus, E protein of West Nile virus, PspA of Streptococcus pneumonia, HSP65 from Mycobacterium tuberculosis, IRP1-3 of Bordetella pertussis, or the heme utilization protein, protective surface antigen D15, heme binding protein A, or outer membrane protein P1, P2, P5 or P6 of Haemophilus influenza.

With regard to other mutant viral segments useful as described herein for a mutant PB2 viral segment, the disclosure provides a mutant PA viral segment with an internal deletion that may encode a protein having the first 190 N-terminal amino acids of PA, which protein may be inhibitory for viral replication, and a mutant PB1 viral segment with an internal deletion that may encode a protein having the first 15 N-terminal amino acids of PB1 as well as residues including amino acids 678 to 757. Thus, DVGs may be obtained from any of the influenza viral segments and these DVGs may also encode inhibitors proteins and be incorporated into VLPs.

### BRIEF DESCRIPTION OF THE FIGURES

FIG. 1. Inhibitory activity of cryptic proteins encoded by a defective viral genome (DVG). Full-length viral genomes produce functional viral proteins (top). The example of the viral polymerase is shown here, where the viral gene encoding PB2 makes one of the three subunits that assemble into a functional polymerase complex. Defective viral genomes (DVGs) are produced during aberrant replication of the viral genome (bottom). Proteins from DVGs of the PB2 gene not only eliminate production of WT PB2, but also yield cryptic proteins that are dominant-negative inhibitors of polymerase assembly, and ultimately viral replication.

FIG. 2. Noncanonical RNA products arise during genome replication (top). DelVGs (DVGs) that maintain their UTRs and packaging signals can be replicated and packaged into virions (middle). Mapping PB2 DelVGs exposes a common breakage region and indicates many DelVGs maintain their open reading frame (bottom).

FIG. 3. Detection of DPRs (deleted proteins) using a PB2-DPR reporter virus.

FIG. 4. PB2 DPRs were cloned from cells infected by the PB2 DPR-V5 reporter virus (top). These DPRs decreased viral polymerase activity when expressed in cells (top) or inhibited viral gene expression when expressed in cells prior to infection (bottom).

FIG. 5. PB2 DPRs competitively inhibit polymerase assembly and impair infection.

FIG. 6. DPRs compete for trimer assembly.

FIG. 7. RNA-Seq shows increased read density at both ends of the PB2 genome segment, confirming the presence of Del VGs in viral stocks (top). Ribo-seq shows a similar pattern as RNA-seq, suggesting DelVGs are actively translated by host ribosomes (middle). Mapping of ribosome-protected fragments from Ribo-Seq reveals translation of deletion-containing mRNAs pinpointing where translation occurs across junctions from both ends of the segment (bottom).

FIG. 8. PB2 DPRs bind PB1 during infection. Upon infection with the PB2-DPR reporter virus, PB1 co-immunoprecipitates with PB2 DPRs (left). PB2 DPRs that inhibit polymerase activity and viral replication (including “a” and “b” here) also individually interact with PB1 (middle and right).

FIG. 9. vRNP and cRNP.

FIG. 10. vRNA, cRNP, svRNA, mvRNA and DelVP.

FIG. 11. Viral genome.

FIG. 12. DelVG.

FIG. 13. Formation of DelVG.

FIG. 14. Formation of DelVG.

FIG. 15. DelVG encode protein products.

FIG. 16. Ribo-Seq identifies canonical linear ribosome protected fragments.

FIG. 17. Comparison of linear ribosome protected fragments versus chimeric ribosome protected fragments produced during translation of DPRs.

FIG. 18. Mapping of PB2 deletions.

FIG. 19. Design of a reporter virus engineered to detect translation of proteins encoded by DelVGs.

FIG. 20. Example of PB2 DelVG-V5 produced by the reporter virus.

FIG. 21. PB2 DPRs produced by the PB2 DelVG-V5 reporter identified by blotting infected cell lysate,

FIG. 22. Co-expression of PB2 DPR clones a-h (blue bars) inhibit polymerase activity.

FIGS. 23A-23B. Expression of PB2 DPR clone a and b inhibit polymerase activity (FIG. 23A) and gene expression in infected cells (FIG. 23B).

FIG. 24. Interference with assembly of polymerase subunits.

FIG. 25. Expression of PB2 DPRs in cells infected with DPR reporter virus (labeled WSN-TRAP).

FIG. 26. PB2 DPR interacts with PB1 as shown by co-precipitation (DPR IP).

FIGS. 27A-27D shows increasing amounts of PB2 DPR compete with WT PB2 during polymerase assembly as revealed by precipitation (PA IP) FIG. 28. DelVGs are translated into proteins.

FIG. 29. DelVGs compete with wild-type PB2 for assembly into trimeric protein.

FIG. 30. PB2 DelVGs inhibit polymerase activity and viral replication.

FIG. 31. Parallel coordinate maps for PB2 from WT virus and one of the PB2-DelVG_V5 viruses (labeled PB2 V5 TRAP).

FIG. 32. Sequences (cRNA) for wild-type WSN virus (SEQ ID Nos. 50-57). SEQ ID NO:50 encodes WSN PB2; SEQ ID NO:51 encodes WSN PB1; SEQ ID NO:52 encodes WSN PA; SEQ ID NO:53 encodes WSN HA; SEQ ID NO:54 encodes WSN NP; SEQ ID NO:55 encodes WSN NA; SEQ ID NO:56 encodes WSN M; and SEQ ID NO:57 encodes WSN NS.

FIG. 33. Coordinates for DVGs for PA, PB1, and NA, and PB2 trap.

FIG. 34. PB2-DelVG-V5 virus composite parallel coordinates

FIG. 35. WT WSN parallel coordinates for PA, PB1, PB2, NP, NS, NA, and M DVGs.

FIG. 36. Potency for cloned PB2-DVGs (SEQ ID NOs: 35-44 and 59-65).

FIGS. 37A-37F. Transcription and translation of deletion-containing viral genomes (DelVGs). FIG. 37A shows reading frames of virions that contain DelVGs with protein-coding potential. FIG. 37B is a bubble plot showing junctions and read depth for all DelVGs. FIG. 37C shows how DelVGs are translated. Ribo-seq data were reanalyzed to allow for discontinuous mapping of ribosome-protected fragments (RPFs). In the top panel, read depth of RPFs are mapped to PB2 in two independent replicates. In the bottom panels, RPFs containing deletion junctions are shown as arcs, where line width reflects read depth. FIG. 37D shows parallel coordinate mapping of PB2 RPFs containing deletion junctions, performed as in FIG. 37A. FIG. 37E is a bubble plot showing that junctions and read depth were determined for all RPFs containing deletions and plotted as a function of their positions from the ends of the respective viral gene. FIG. 37F is a bubble plot showing the abundance of deletion-containing fragments from PB2 is correlated between total RNA and RPFs.

FIGS. 38A-38D show the expression of DelVG-encoded proteins during infection. FIG. 38A is a diagram of the PB2 gene in a reporter virus designed to capture expression of DelVG-encoded proteins. FIG. 38B shows parallel coordinate mapping of DelVGs identified by sequencing the PB2 TRAP V5 stock #1 DelVGs with the potential to express V5-tagged proteins (top) and the remaining DelVGs (bottom) are shown. FIG. 38C is a bubble plot showing that junctions and read depth were determined for all DelVGs in three independent plaque-purified stocks and plotted as a function of their positions from the ends of the respective viral gene. Note that none of the DelVGs in this sample mapped to NS. FIG. 38D is a western blot showing expression of DelVG-encoded proteins (DPRs) in infected cells.

FIGS. 39A-39D show that DPRs compete for assembly of function viral polymerase. FIG. 39A shows DPRs and co-precipitating proteins detected by western blot. Lysate were prepared from cells infected with the PB2 TRAP V5 virus and DPRs were immunopurified via their V5 tag. FIG. 39B shows the viral polymerase proteins and NP were expressed in the presence or absence of the indicated DPR. DPRs were immunopurified from cell lysates and proteins were detected by western blot. FIG. 39C shows that PB2 DPR competes for polymerase assembly. The viral polymerase proteins and NP were expressed in the presence of increasing amounts of the PB2 DPR. Polymerase assembly was measured by immunoprecipitating PA and probing for PB2. FIG. 39D is a schematic illustrating a dual mechanism for the inhibitory activity for DelVGs and the DPRs they encode.

FIGS. 40A-40F show that DPRs are dominant-negative inhibitors of the influenza virus polymerase and viral replication. FIG. 40A is a table showing DelVGs cloned from cells infected with the PB2 TRAP V5 reporter virus. Deletion junctions were mapped back to WT PB2 and used to name each DelVG Numbering is based on full-length PB2 cRNA and indicates the last nucleotide of the upstream portion and the first nucleotide of the downstream sequence. FIG. 40B is a graph depicting polymerase activity assays that were performed in 293T cells in the presence or absence of the indicated DPRs. PB1, PB2, PA, NP and DPRs are all derived from WSN. FIG. 40C is a graph depicting polymerase activity assays that were performed as in FIG. 40B and in the presence of PB2 416/2189 or a version containing premature stop codons. DPR expression was detected by western blot. FIG. 40D shows DPR sequence (top, SEQ ID NO. 66) and a graph of polymerase interference by heterotypic DPRs. Polymerase activity assays in the presence or absence of the PB2 416/2189 DPR were performed as in FIG. 40B, expect the DPR was cloned from the indicated viral strain. FIGS. 40E and 40F show that DPRs suppress viral replication. FIG. 40E is a graph of the indicated DPRs that were ectopically expressed in cells prior to infection with an influenza reporter virus. Supernatants were harvested 24 hpi and viral titers were measured. DPR expression was assessed by western blot (lower panel). FIG. 40F is a graph showing replication of the wild-type virus was measured by tittering the supernatant via a luciferase-based assay. Cells were co-infected with a wild-type reporter virus and a clonal virus encoding the indicated DPR in place of PB2.

FIGS. 41A and 41B show the deletion-containing viral genomes (DelVGs) from viral stock from FIGS. 37A and 37B. FIG. 41A depicts parallel coordinate mapping of DelVG that were identified by sequencing genomic RNA purified from WSN virions. Genes are shown as plus-sense cRNA, and each DelVGs is depicted by a line connecting the 5′ (top) and 3′ (bottom) ends of each deletion. FIG. 41B is a bubble plot showing junctions and read depth determined for all DelVGs and plotted as a function of their positions from the ends of the respective viral gene. These are the same data as in FIG. 37B and scaled to show the full length of the gene segment. Note that none of the DelVGs in this sample mapped to HA.

FIGS. 42A to 42E show Ribo-seq data that reveals transcription and translation of deletion-containing viral genomes (DelVGs) from FIGS. 37C-37F. FIG. 42A shows Ribo-seq data reanalyzed to allow for discontinuous mapping of ribosome-protected fragments (RPFs). Genes are shown as plus-sense cRNA, and each DelVGs is depicted by a line connecting the 5′ (top) and 3′ (bottom) ends of each deletion. FIG. 42B shows that DelVGs were identified in the total RNA input controls from Ribo-Seq and mapped as in FIG. 37A. PB2 was further separated based on predicted reading frames downstream of the deletion. FIG. 42C shows predicted protein length for DelVGs detected in total RNA and RPFs. FIG. 42D is a bubble plot showing junctions and read depth that were determined for all RPFs containing a deletion and plotted as a function of their positions from the ends of the respective viral gene. These are the same data as in FIG. 37E and scaled to show the full length of the gene segment. FIG. 42E is a bubble plot showing junctions and read depth were determined for all DelVG in total RNA input controls and plotted as a function of their positions from the ends of the respective viral gene.

FIGS. 43A and 43B are parallel coordinates for DelVGs detected in human challenge studies. FIG. 43A shows parallel coordinates for DelVGs detected in humans challenged with A/Wisconsin/67/2005 (H3N2). FIG. 43B shows parallel coordinates for DelVGs detected in humans naturally infected with A/Anhui/1/2013 (H7N9).

FIGS. 44A-44B are parallel coordinates for PB2 TRAP V5 reporter virus as shown in FIGS. 38A-38C. FIG. 44A are parallel coordinates for DelVGs identified by sequencing genomic RNA purified from virions of three plaque-purified stocks of the PB2 TRAP V5 reporter virus. Data from each sample were consolidated and visualized by parallel coordinate mapping. Genes are shown as plus-sense cRNA. Each DelVG is depicted by a line connecting the 5′ (top) and 3′ (bottom) ends of each deletion. FIG. 44B is a bubble plot showing junctions and read depth determined for all DelVGs and plotted as a function of their positions from the ends of the respective viral gene. These are the same data as in FIG. 38C and scaled to show the full length of the gene segment. Note that none of the DelVGs in this sample mapped to NS.

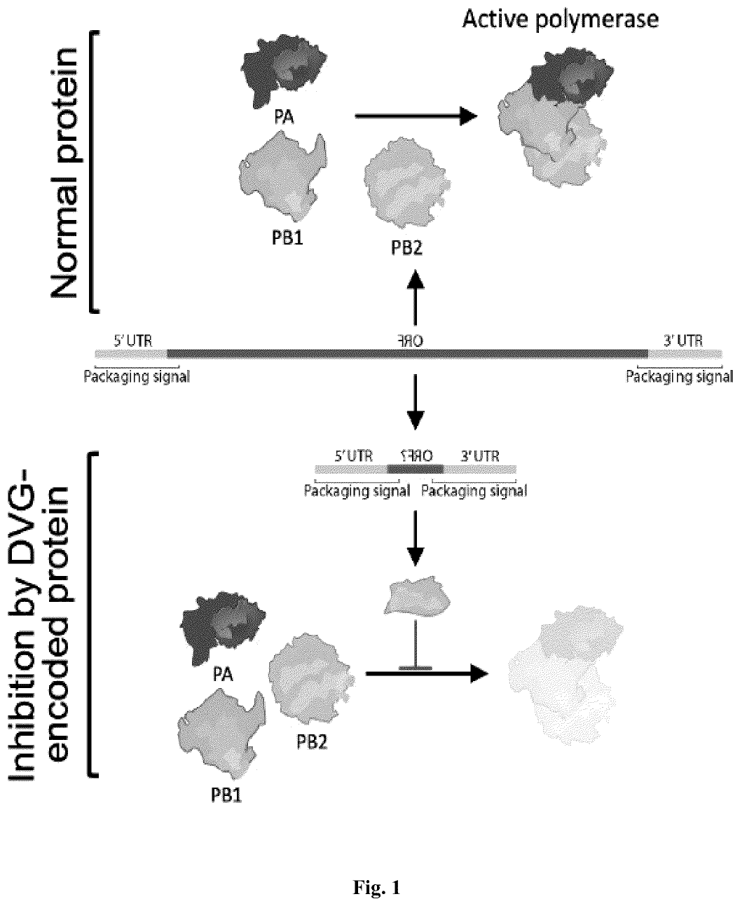

### DETAILED DESCRIPTION

DIPs (defective interfering particles, formed by incorporating DVGs into new virions) are known to block replication of their WT counterparts. It has been assumed that this was achieved by the DVG RNA itself. As disclosed herein, proteins encoded by DVGs also contribute to the inhibitory activity. This reveals new inhibitory mechanisms and provides additional opportunities to specifically engineer DIPs to block WT virus replication, to serve as immunostimulatory agents, or use as attenuated vaccine backbones. DIPs thus offer a ‘one-two punch’ where both the RNA itself and the encoded protein coordinate to interfere with replication. The immunogenicity of the DVG in the DIP is enhanced relative to WT.

WT full-length genomes can be immunogenic but they are normally coated by viral proteins and this prevents sensing by the cell, muting their immunogenicity. DVGs are much more abundant than full-length. That alone increases their immunogenicity. In some cases, they can be replicated without being coated by viral proteins. This means their immune signature is fully unmasked, thus enhancing their immunogenicity.

Therefore, viruses can be generated to produce engineered DIPs with dual function, coding for both inhibitory proteins and parasitic RNAs. These can be potent competitors during infection suppressing replication of WT virus, offering therapeutic potential against a diverse array of influenza viruses. In addition to engineering viruses with DVGs encoding inhibitory proteins, it may be beneficial to suppress protein production or their inhibitory function. DIPs arise naturally in viral populations. They are known to contaminate influenza virus stocks used for vaccine production. The inhibitory activity for DVGs and their encoded proteins identifies an additional parameter that can be optimized to increase viral yield. This also provides the basis for strategies to minimize DVG protein production or inhibitory activity, which could increase vaccine yield. The immunogenicity of the vaccine may also be altered by changing the composition of DIPs in the population and their protein coding potential.

The DVGs may delivered by particles including viral particles, e.g., virus-like particles (VLPs), or as an mRNA for direct translation. The disclosed proteins encoded by the DVGs may delivered in any delivery vehicle, e.g., a sustained release delivery vehicle or as a vector encoding the protein, which protein or vector may be present in compositions including polymers, e.g., synthetic polymers, or liposomes.

### Definitions

As used herein, the term “isolated” refers to in vitro preparation and/or isolation of a nucleic acid molecule, e.g., vector or plasmid, peptide or polypeptide (protein), or virus of the disclosure, so that it is not associated with in vivo substances, or is substantially purified from in vitro substances. An isolated virus preparation is generally obtained by in vitro culture and propagation, and/or via passage in eggs, and is substantially free from other infectious agents.

As used herein, “substantially purified” means the object species is the predominant species, e.g., on a molar basis it is more abundant than any other individual species in a composition, and preferably is at least about 80% of the species present, and optionally 90% or greater, e.g., 95%, 98%, 99% or more, of the species present in the composition.

As used herein, “substantially free” means below the level of detection for a particular infectious agent using standard detection methods for that agent.

A “recombinant” virus is one which has been manipulated in vitro, e.g., using recombinant DNA techniques, to introduce changes to the viral genome. Reassortant viruses can be prepared by recombinant or nonrecombinant techniques.

As used herein, the term “recombinant nucleic acid” or “recombinant DNA sequence or segment” refers to a nucleic acid, e.g., to DNA, that has been derived or isolated from a source, that may be subsequently chemically altered in vitro so that its sequence is not naturally occurring or corresponds to naturally occurring sequences that are not positioned as they would be positioned in the native genome. An example of DNA “derived” from a source, would be a DNA sequence that is identified as a useful fragment, and which is then chemically synthesized in essentially pure form. An example of such DNA “isolated” from a source would be a useful DNA sequence that is excised or removed from said source by chemical means, e.g., by the use of restriction endonucleases, so that it can be further manipulated, e.g., amplified, for use in the invention, by the methodology of genetic engineering.

As used herein, a “heterologous” nucleotide sequence is from a source other than a parent influenza virus, e.g., a reporter gene or a gene from another virus or organism, e.g., a bacterium, or is from an influenza virus source but is in a context that does not mimic a native influenza virus genome, e.g., it is a subset of a full length influenza virus gene segment and is in a non-native context, e.g., fused to truncated PB2 coding sequences.

As used herein, a “heterologous” influenza virus gene or gene segment is from an influenza virus source that is different than a majority of the other influenza viral genes or gene segments in a recombinant, e.g., reassortant, influenza virus.

The terms “isolated polypeptide”, “isolated peptide” or “isolated protein” include a polypeptide, peptide or protein encoded by cDNA or recombinant RNA including one of synthetic origin, or some combination thereof.

The term “recombinant protein” or “recombinant polypeptide” as used herein refers to a protein molecule expressed from a recombinant DNA molecule. In contrast, the term “native protein” is used herein to indicate a protein isolated from a naturally occurring (i.e., a nonrecombinant) source. Molecular biological techniques may be used to produce a recombinant form of a protein with identical properties as compared to the native form of the protein.

A “vector” refers to a macromolecule or association of macromolecules that comprises or associates with a polynucleotide, and which can be used to mediate delivery of the polynucleotide to a cell, either in vitro or in vivo. Illustrative vectors include, for example, plasmids, viral vectors, liposomes and other gene delivery vehicles. The polynucleotide to be delivered, sometimes referred to as a “target polynucleotide” or “transgene,” may comprise a coding sequence of interest in gene therapy (such as a gene encoding a protein of therapeutic interest), a coding sequence of interest in vaccine development (such as a polynucleotide expressing a protein, polypeptide or peptide suitable for eliciting an immune response in a mammal), and/or a selectable or detectable marker.

“Transduction,” “transfection,” “transformation” or “transducing” as used herein, are terms referring to a process for the introduction of an exogenous polynucleotide into a host cell leading to expression of the polynucleotide, e.g., the transgene in the cell, and includes the use of recombinant virus to introduce the exogenous polynucleotide to the host cell. Transduction, transfection or transformation of a polynucleotide in a cell may be determined by methods well known to the art including, but not limited to, protein expression (including steady state levels), e.g., by ELISA, flow cytometry and Western blot, measurement of DNA and RNA by hybridization assays, e.g., Northern blots, Southern blots and gel shift mobility assays. Methods used for the introduction of the exogenous polynucleotide include well-known techniques such as viral infection or transfection, lipofection, transformation and electroporation, as well as other non-viral gene delivery techniques. The introduced polynucleotide may be stably or transiently maintained in the host cell.

“Gene delivery” refers to the introduction of an exogenous polynucleotide into a cell for gene transfer, and may encompass targeting, binding, uptake, transport, localization, replicon integration and expression.

“Gene transfer” refers to the introduction of an exogenous polynucleotide into a cell which may encompass targeting, binding, uptake, transport, localization and replicon integration, but is distinct from and does not imply subsequent expression of the gene.

“Gene expression” or “expression” refers to the process of gene transcription, translation, and post-translational modification.

An “infectious” virus or viral particle is one that comprises a polynucleotide component which it is capable of delivering into a cell for which the viral species is trophic. The term does not necessarily imply any replication capacity of the virus.

A “VLP” as used herein is an influenza virus-like particle containing the full-complement of viral gene segments, e.g., 8 gene segments for influenza A and B viruses or 7 gene segments for influenza C viruses. A VLP can infect a cell but by itself cannot produce progeny virus unless something is provided in trans, e.g., by providing wild-type virus or one or more viral proteins. In one embodiment, the VLP has 8 genomic segments (for influenza A and B viruses) and each of those influenza A virus or influenza B virus segments except one has sequences that encode functional influenza virus protein(s). In one embodiment, the VLP has 7 genomic segments (for influenza C viruses) and each of those influenza C virus segments but one has sequences that encode functional influenza virus protein(s). In one embodiment, at least one influenza viral genomic segment may not encode a functional version of the main viral protein(s) encoded by the respective segment (i.e., the PB2, PB1, PA, HA, NP, NA, M1, M2, NS1, NS2 proteins), e.g., as a result of modification of the genomic segment, for instance, via deletion, insertion or substitution of one or more nucleotides. In one embodiment, a VLP may have the same number of viral segments as a wild-type influenza virus, e.g., for influenza A or B that is 8 segments, but at least one segment in the VLP is non-functional, e.g., does not express a viral protein for replication or packaging, or for infectious virus production. In one embodiment, the VIP has 8 genomic segments (for influenza A and B viruses) or 7 gene segments (for influenza C virus), where one of the gene segments is replaced with a DVG. In one embodiment, the VLP has 8 genomic segments (for influenza A and B viruses) or 7 gene segments (for influenza C virus), where one of the gene segments is replaced with a DVG that encodes an inhibitory protein. In one embodiment, the VLP may comprise at least a protein required for binding to host cells (for example, the influenza virus HA protein, the Ebola virus GP protein, or the VSV G protein), and the viral proteins required for viral RNA replication and transcription (i.e., the PB2, PB1, PA, and NP proteins which, together with the viral RNA, form the viral ribonucleoprotein complex). Most VLPs will be replication incompetent (i.e., dependent on a helper function). Some VLPs may be replication competent (i.e., they may lack the capability to express an influenza viral protein such as NA or NS1, but may still be able to form infectious progeny viruses). A replication competent VLP may be attenuated.

The terms “nucleic acid,” “polynucleotide,” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

An “isolated” polynucleotide, e.g., plasmid, virus, polypeptide, cell, or other substance refers to a preparation of the substance devoid of at least some of the other components that may also be present where the substance or a similar substance naturally occurs or is initially prepared from. Thus, for example, an isolated substance may be prepared by using a purification technique to enrich it from a source mixture. Isolated nucleic acid, peptide or polypeptide is present in a form or setting that is different from that in which it is found in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. The isolated nucleic acid molecule may be present in single-stranded or double-stranded form. When an isolated nucleic acid molecule is to be utilized to express a protein, the molecule will contain at a minimum the sense or coding strand (i.e., the molecule may single-stranded), but may contain both the sense and antisense strands (i.e., the molecule may be double-stranded). Enrichment can be measured on an absolute basis, such as weight per volume of solution, or it can be measured in relation to a second, potentially interfering substance present in the source mixture. Increasing enrichments of the embodiments of this invention are envisioned. Thus, for example, a 2-fold enrichment, 10-fold enrichment, 100-fold enrichment, or a 1000-fold enrichment.

A “transcriptional regulatory sequence” refers to a genomic region that controls the transcription of a gene or coding sequence to which it is operably linked. Transcriptional regulatory sequences of use in the present invention generally include at least one transcriptional promoter and may also include one or more enhancers and/or terminators of transcription.

“Operably linked” refers to an arrangement of two or more components, wherein the components so described are in a relationship permitting them to function in a coordinated manner. By way of illustration, a transcriptional regulatory sequence or a promoter is operably linked to a coding sequence if the TRS or promoter promotes transcription of the coding sequence. An operably linked TRS is generally joined in cis with the coding sequence, but it is not necessarily directly adjacent to it.

“Heterologous” means derived from a genotypically distinct entity from the entity to which it is compared. For example, a polynucleotide introduced by genetic engineering techniques into a different cell type is a heterologous polynucleotide (and, when expressed, can encode a heterologous polypeptide). Similarly, a transcriptional regulatory element such as a promoter that is removed from its native coding sequence and operably linked to a different coding sequence is a heterologous transcriptional regulatory element.

A “terminator” refers to a polynucleotide sequence that tends to diminish or prevent read-through transcription (i.e., it diminishes or prevent transcription originating on one side of the terminator from continuing through to the other side of the terminator). The degree to which transcription is disrupted is typically a function of the base sequence and/or the length of the terminator sequence. In particular, as is well known in numerous molecular biological systems, particular DNA sequences, generally referred to as “transcriptional termination sequences” are specific sequences that tend to disrupt read-through transcription by RNA polymerase, presumably by causing the RNA polymerase molecule to stop and/or disengage from the DNA being transcribed. Typical example of such sequence-specific terminators include polyadenylation (“poly A”) sequences, e.g., SV40 polyA. In addition to or in place of such sequence-specific terminators, insertions of relatively long DNA sequences between a promoter and a coding region also tend to disrupt transcription of the coding region, generally in proportion to the length of the intervening sequence. This effect presumably arises because there is always some tendency for an RNA polymerase molecule to become disengaged from the DNA being transcribed, and increasing the length of the sequence to be traversed before reaching the coding region would generally increase the likelihood that disengagement would occur before transcription of the coding region was completed or possibly even initiated. Terminators may thus prevent transcription from only one direction (“uni-directional” terminators) or from both directions (“bi-directional” terminators), and may be comprised of sequence-specific termination sequences or sequence-non-specific terminators or both. A variety of such terminator sequences are known in the art; and illustrative uses of such sequences within the context of the present invention are provided below.

“Host cells,” “cell lines,” “cell cultures,” “packaging cell line” and other such terms denote higher eukaryotic cells, such as mammalian cells including human cells, useful in the present invention, e.g., to produce recombinant virus or recombinant fusion polypeptide. These cells include the progeny of the original cell that was transduced. It is understood that the progeny of a single cell may not necessarily be completely identical (in morphology or in genomic complement) to the original parent cell.

“Recombinant,” as applied to a polynucleotide means that the polynucleotide is the product of various combinations of cloning, restriction and/or ligation steps, and other procedures that result in a construct that is distinct from a polynucleotide found in nature. In one embodiment, a recombinant virus is a viral particle comprising a recombinant polynucleotide. The terms respectively include replicates of the original polynucleotide construct and progeny of the original virus construct.

A “control element” or “control sequence” is a nucleotide sequence involved in an interaction of molecules that contributes to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers. A promoter is a DNA region capable under certain conditions of binding RNA polymerase and initiating transcription of a coding region usually located downstream (in the 3′ direction) from the promoter. Promoters include heterologous viral promoters such as a CMV promoter or AAV promoters, e.g., P5, P19, P40 and AAV ITR promoters, as well as heterologous non-viral promoters.

An “expression vector” is a vector comprising a region which encodes a gene product of interest, and is used for effecting the expression of the gene product in an intended target cell. An expression vector also comprises control elements operatively linked to the encoding region to facilitate expression of the protein in the target. The combination of control elements and a gene or genes to which they are operably linked for expression is sometimes referred to as an “expression cassette,” a large number of which are known and available in the art or can be readily constructed from components that are available in the art.

The terms “polypeptide” and “protein” are used interchangeably herein to refer to polymers of amino acids of any length. The terms also encompass an amino acid polymer that has been modified; for example, disulfide bond formation, glycosylation, acetylation, phosphorylation, lipidation, or conjugation with a labeling component.

The term “exogenous,” when used in relation to a protein, gene, nucleic acid, or polynucleotide in a cell or organism refers to a protein, gene, nucleic acid, or polynucleotide which has been introduced into the cell or organism by artificial or natural means. An exogenous nucleic acid may be from a different organism or cell, or it may be one or more additional copies of a nucleic acid which occurs naturally within the organism or cell. By way of a non-limiting example, an exogenous nucleic acid is in a chromosomal location different from that of natural cells, or is otherwise flanked by a different nucleic acid sequence than that found in nature, e.g., an expression cassette which links a promoter from one gene to an open reading frame for a gene product from a different gene.

“Transformed” or “transgenic” is used herein to include any host cell or cell line, which has been altered or augmented by the presence of at least one recombinant DNA sequence. The host cells of the present invention are typically produced by transfection with a DNA sequence in a plasmid expression vector, as an isolated linear DNA sequence, or infection with a recombinant viral vector.

The term “sequence homology” means the proportion of base matches between two nucleic acid sequences or the proportion amino acid matches between two amino acid sequences. When sequence homology is expressed as a percentage, e.g., 50%, the percentage denotes the proportion of matches over the length of a selected sequence that is compared to some other sequence. Gaps (in either of the two sequences) are permitted to maximize matching; gap lengths of 15 bases or less are usually used, 6 bases or less e.g., with 2 bases or less. When using oligonucleotides as probes or treatments, the sequence homology between the target nucleic acid and the oligonucleotide sequence is generally not less than 17 target base matches out of 20 possible oligonucleotide base pair matches (85%); not less than 9 matches out of 10 possible base pair matches (90%), or not less than 19 matches out of 20 possible base pair matches (95%).

Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less or with 2 or less. Alternatively, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. The two sequences or parts thereof are more homologous if their amino acids are greater than or equal to 50% identical when optimally aligned using the ALIGN program.

The term “corresponds to” is used herein to mean that a polynucleotide sequence is structurally related to all or a portion of a reference polynucleotide sequence, or that a polypeptide sequence is structurally related to all or a portion of a reference polypeptide sequence, e.g., they have at least 80%, 85%, 90%, 95% or more, e.g., 99% or 100%, sequence identity. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

The term “sequence identity” means that two polynucleotide sequences are identical (i.e., on a nucleotide-by-nucleotide basis) over the window of comparison. The term “percentage of sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denote a characteristic of a polynucleotide sequence, wherein the polynucleotide comprises a sequence that has at least 85 percent sequence identity, e.g., at least 90 to 95 percent sequence identity, or at least 99 percent sequence identity as compared to a reference sequence over a comparison window of at least 20 nucleotide positions, frequently over a window of at least 20-50 nucleotides, wherein the percentage of sequence identity is calculated by comparing the reference sequence to the polynucleotide sequence which may include deletions or additions which total 20 percent or less of the reference sequence over the window of comparison.

“Conservative” amino acid substitutions are, for example, aspartic-glutamic as polar acidic amino acids; lysine/arginine/histidine as polar basic amino acids; leucine/isoleucine/methionine/valine/alanine/glycine/proline as non-polar or hydrophobic amino acids; serine/threonine as polar or uncharged hydrophilic amino acids. Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting polypeptide. Whether an amino acid change results in a functional polypeptide can readily be determined by assaying the specific activity of the polypeptide. Naturally occurring residues are divided into groups based on common side-chain properties: (1) hydrophobic: norleucine, met, ala, val, leu, ile; (2) neutral hydrophilic: cys, ser, thr; (3) acidic: asp, glu; (4) basic: asn, gln, his, lys, arg; (5) residues that influence chain orientation: gly, pro; and (6) aromatic; trp, tyr, phe.

The disclosure also envisions polypeptides with non-conservative substitutions. Non-conservative substitutions entail exchanging a member of one of the classes described above for another.

As used herein, “individual” (as in the subject of the treatment) means a mammal. Mammals include, for example, humans; non-human primates, e.g., apes and monkeys; and non-primates, e.g., dogs, cats, rats, mice, cattle, horses, sheep, and goats. Non-mammals include, for example, fish and birds.

The term “disease” or “disorder” are used interchangeably, and are used to refer to diseases or conditions wherein lack of or reduced amounts of a specific gene product, e.g., a lysosomal storage enzyme, plays a role in the disease such that a therapeutically beneficial effect can be achieved by supplementing, e.g., to at least 1% of normal levels.

“Substantially” as the term is used herein means completely or almost completely; for example, a composition that is “substantially free” of a component either has none of the component or contains such a trace amount that any relevant functional property of the composition is unaffected by the presence of the trace amount, or a compound is “substantially pure” is there are only negligible traces of impurities present.

“Treating” or “treatment” within the meaning herein refers to an alleviation of symptoms associated with a disorder or disease, “inhibiting” means inhibition of further progression or worsening of the symptoms associated with the disorder or disease, and “preventing” refers to prevention of the symptoms associated with the disorder or disease.

The term “sequence” refers to a nucleotide sequence of any length, which can be DNA or RNA; can be linear, circular or branched and can be either single-stranded or double stranded. The term “donor sequence” refers to a nucleotide sequence that is inserted into a genome. A donor sequence can be of any length, for example between 2 and 10,000 nucleotides in length (or any integer value therebetween or thereabove), e.g., between about 100 and 1,000 nucleotides in length (or any integer therebetween), e.g., between about 200 and 500 nucleotides in length.

For example, an exogenous nucleic acid can comprise an infecting viral genome, a plasmid or episome introduced into a cell, or a chromosome that is not normally present in the cell. Methods for the introduction of exogenous molecules into cells are known to those of skill in the art and include, but are not limited to, lipid-mediated transfer (e.g., liposomes, including neutral and cationic lipids), electroporation, direct injection, cell fusion, particle bombardment, calcium phosphate co-precipitation, DEAE-dextran-mediated transfer and viral vector-mediated transfer. An exogenous molecule can also be the same type of molecule as an endogenous molecule but derived from a different species than the cell is derived from. For example, a human nucleic acid sequence may be introduced into a cell line originally derived from a mouse or hamster.

### Influenza Virus

The life cycle of viruses generally involves attachment to cell surface receptors, entry into the cell and uncoating of the viral nucleic acid, followed by replication of the viral genes inside the cell. After the synthesis of new copies of viral proteins and genes, these components assemble into progeny virus particles, which then exit the cell (reviewed by Roizman and Palese, 1996). Different viral proteins play a role in each of these steps.

The influenza A virus is an enveloped negative-strand virus with eight RNA segments encapsidated with nucleoprotein (NP) (reviewed by Lamb and Krug, 1996). The eight single-stranded negative-sense viral RNAs (vRNAs) encode a total of ten to eleven proteins. The influenza virus life cycle begins with binding of the hemagglutinin (HA) to sialic acid-containing receptors on the surface of the host cell, followed by receptor-mediated endocytosis. The low pH in late endosomes triggers a conformational shift in the HA, thereby exposing the N-terminus of the HA2 subunit (the so-called fusion peptide). The fusion peptide initiates the fusion of the viral and endosomal membrane, and the matrix protein (M1) and RNP complexes are released into the cytoplasm. RNPs consist of the nucleoprotein (NP), which encapsidates vRNA, and the viral polymerase complex, which is formed by the PA, PB1, and PB2 proteins. RNPs are transported into the nucleus, where transcription and replication take place. The RNA polymerase complex catalyzes three different reactions: synthesis of an mRNA with a S′ cap and 3′ polyA structure, of a full-length complementary RNA (cRNA), and of genomic vRNA using the cDNA as a template. Newly synthesized vRNAs, NP, and polymerase proteins are then assembled into RNPs, exported from the nucleus, and transported to the plasma membrane, where budding of progeny virus particles occurs. The neuraminidase (NA) protein plays a crucial role late in infection by removing sialic acid from sialyloligosaccharides, thus releasing newly assembled virions from the cell surface and preventing the self aggregation of virus particles. Although virus assembly involves protein-protein and protein-vRNA interactions, the nature of these interactions is largely unknown.

Although influenza B and C viruses are structurally and functionally similar to influenza A virus, there are some differences. For example, the M segment of influenza B virus encodes two proteins, M1 and BM2, through a termination-reinitiation scheme of tandem cistrons, and the NA segment encodes the NA and NB proteins from a bicistronic mRNA. Influenza C virus, which has 7 vRNA segments, relies on spliced transcripts to produce M1 protein; the product of the unspliced mRNA is proteolytically cleaved to yield the CM2 protein. In addition, influenza C virus encodes a HA-esterase (HEF) rather than individual HA and NA proteins.

Spanning the viral membrane for influenza A virus are three proteins: hemagglutinin (HA), neuraminidase (NA), and M2. The extracellular domains (ectodomains) of HA and NA are quite variable, while the ectodomain domain of M2 is essentially invariant among influenza A viruses. The M2 protein which possesses ion channel activity (Pinto et al., 1992), is thought to function at an early state in the viral life cycle between host cell penetration and uncoating of viral RNA (Martin and Helenius, 1991; reviewed by Helenius, 1992; Sugrue et al., 1990). Once virions have undergone endocytosis, the virion-associated M2 ion channel, a homotetrameric helix bundle, is believed to permit protons to flow from the endosome into the virion interior to disrupt acid-labile M1 protein-ribonucleoprotein complex (RNP) interactions, thereby promoting RNP release into the cytoplasm (reviewed by Helenius, 1992). In addition, among some influenza strains whose HAs are cleaved intracellularly (e.g., A/fowl plagues/Rostock/34), the M2 ion channel is thought to raise the pH of the trans-Golgi network, preventing conformational changes in the HA due to conditions of low pH in this compartment (Hay et al., 1985; Ohuchi et al., 1994; Takeuchi and Lamb, 1994).

Cells that can be Used to Prepare Influenza Viruses

Any cell, e.g., any avian or mammalian cell, such as a human, e.g., 293T or PER.C6® cells, or canine, bovine, equine, feline, swine, ovine, rodent, for instance mink, e.g., MvLu1 cells, or hamster, e.g., CHO cells, non-human primate, e.g., Vero cells, or non-primate higher vertebrate cells, e.g., MDCK cells, including mutant cells such as AX4 cells, which support efficient replication of influenza virus can be employed to isolate and/or propagate influenza viruses or VLPs. Isolated viruses can be used to prepare a reassortant virus. In one embodiment, host cells for vaccine production are continuous mammalian or avian cell lines or cell strains. A complete characterization of the cells to be used, may be conducted so that appropriate tests for Data that can be used for the purity of the final product can be included, characterization of a cell includes (a) information on its origin, derivation, and passage history; (b) information on its growth and morphological characteristics; (c) results of tests of adventitious agents; (d) distinguishing features, such as biochemical, immunological, and cytogenetic patterns which allow the cells to be clearly recognized among other cell lines; and (e) results of tests for tumorigenicity. In one embodiment, the passage level, or population doubling, of the host cell used is as low as possible.

In one embodiment, the cells are WHO certified, or certifiable, continuous cell lines. The requirements for certifying such cell lines include characterization with respect to at least one of genealogy, growth characteristics, immunological markers, virus susceptibility tumorigenicity and storage conditions, as well as by testing in animals, eggs, and cell culture. Such characterization is used to confirm that the cells are free from detectable adventitious agents. In some countries, karyology may also be required. In addition, tumorigenicity may be tested in cells that are at the same passage level as those used for vaccine production. The virus may be purified by a process that has been shown to give consistent results, before vaccine production (see, e.g., World Health Organization, 1982).

Virus produced by the host cell may be highly purified prior to vaccine or gene therapy formulation. Generally, the purification procedures result in extensive removal of cellular DNA and other cellular components, and adventitious agents. Procedures that extensively degrade or denature DNA may also be used.

### Influenza Vaccines

A vaccine includes an isolated recombinant influenza virus, and optionally one or more other isolated viruses including other isolated influenza viruses, one or more immunogenic proteins or glycoproteins of one or more isolated influenza viruses or one or more other pathogens, e.g., an immunogenic protein from one or more bacteria, non-influenza viruses, yeast or fungi, or isolated nucleic acid encoding one or more viral proteins (e.g., DNA or RNA vaccines) including one or more proteins of the isolated influenza virus described herein. In one embodiment, the influenza viruses described herein may be vaccine vectors for influenza virus or other pathogens.

A subunit vaccine comprises purified glycoproteins. Such a vaccine may be prepared as follows: using viral suspensions fragmented by treatment with detergent, the surface antigens are purified, by ultracentrifugation for example. The subunit vaccines thus contain mainly HA protein, and also NA. The detergent used may be cationic detergent for example, such as hexadecyl trimethyl ammonium bromide (Bachmeyer, 1975), an anionic detergent such as ammonium deoxycholate (Laver & Webster, 1976); or a nonionic detergent such as that commercialized under the name TRITON X100. The hemagglutinin may also be isolated after treatment of the virions with a protease such as bromelin, and then purified. The subunit vaccine may be combined with an attenuated virus of the invention in a multivalent vaccine.

A split vaccine comprises virions which have been subjected to treatment with agents that dissolve lipids. A split vaccine can be prepared as follows: an aqueous suspension of the purified virus obtained as above, inactivated or not, is treated, under stirring, by lipid solvents such as ethyl ether or chloroform, associated with detergents. The dissolution of the viral envelope lipids results in fragmentation of the viral particles. The aqueous phase is recuperated containing the split vaccine, constituted mainly of hemagglutinin and neuraminidase with their original lipid environment removed, and the core or its degradation products. Then the residual infectious particles are inactivated if this has not already been done. The split vaccine may be combined with an attenuated virus of the invention in a multivalent vaccine.

Inactivated influenza virus vaccines are provided by inactivating replicated virus using known methods, such as, but not limited to, formalin or β-propiolactone treatment. Inactivated vaccine types that can be used in the invention can include whole-virus (WV) vaccines or subvirion (SV) (split) vaccines. The WV vaccine contains intact, inactivated virus, while the SV vaccine contains purified virus disrupted with detergents that solubilize the lipid-containing viral envelope, followed by chemical inactivation of residual virus.

In addition, vaccines that can be used include those containing the isolated HA and NA surface proteins, which are referred to as surface antigen or subunit vaccines.

Live, attenuated influenza virus vaccines can be used for preventing or treating influenza virus infection.

The viruses in a multivalent vaccine can include attenuated or inactivated viruses, formulated and administered, according to known methods, as a vaccine to induce an immune response in an animal, e.g., a mammal.

### Pharmaceutical Compositions

Pharmaceutical compositions of the present disclosure, suitable for inoculation, e.g., nasal, parenteral or oral administration, comprise one or more influenza virus isolates, isolated nucleic acid, e.g., a vector that encodes a protein, a VLP having nucleic acid that encodes a protein described herein, isolated protein, or any combination thereof, optionally further comprising sterile aqueous or non-aqueous solutions, suspensions, and emulsions. The compositions can further comprise auxiliary agents or excipients, as known in the art. The composition of the disclosure is generally presented in the form of individual doses (unit doses).

Conventional influenza vaccines generally contain about 0.1 to 200 μg, e.g., 30 to 100 μg, of HA from each of the strains entering into their composition. The vaccine forming the main constituent of a composition may comprise a single influenza virus, or a combination of influenza viruses, for example, at least two or three influenza viruses, including one or more reassortant(s).

Preparations for parenteral administration include sterile aqueous or non-aqueous solutions, suspensions, and/or emulsions, which may contain auxiliary agents or excipients known in the art. Examples of non-aqueous solvents are propylene glycol, polyethylene glycol, vegetable oils such as olive oil, and injectable organic esters such as ethyl oleate. Carriers or occlusive dressings can be used to increase skin permeability and enhance antigen absorption. Liquid dosage forms for oral administration may generally comprise a liposome solution containing the liquid dosage form. Suitable forms for suspending liposomes include emulsions, suspensions, solutions, syrups, and elixirs containing inert diluents commonly used in the art, such as purified water. Besides the inert diluents, such compositions can also include adjuvants, wetting agents, emulsifying and suspending agents, or sweetening, flavoring, or perfuming agents.

When a composition is used for administration to an individual, it can further comprise salts, buffers, adjuvants, or other substances which are desirable for improving the efficacy of the composition. For vaccines, adjuvants, substances which can augment a specific immune response, can be used. Normally, the adjuvant and the composition are mixed prior to presentation to the immune system, or presented separately, but into the same site of the organism being immunized.

Heterogeneity in a vaccine may be provided by mixing immunogens such as at least two influenza virus strains, such as 2-20 strains or any range or value therein, or a virus that provides for multivalency. Vaccines can be provided for variations in a single strain of an influenza virus, using techniques known in the art.

A pharmaceutical composition may further or additionally comprise at least one chemotherapeutic compound, for example, immunosuppressants, anti-inflammatory agents or immune enhancers, and for vaccines, chemotherapeutics including, but not limited to, gamma globulin, amantadine, guanidine, hydroxybenzimidazole, interferon-α, interferon-β, interferon-γ, tumor necrosis factor-alpha, thiosemicarbarzones, methisazone, rifampin, ribavirin, a pyrimidine analog, a purine analog, foscarnet, phosphonoacetic acid, acyclovir, dideoxynucleosides, a protease inhibitor, or ganciclovir.

The composition can also contain variable but small quantities of endotoxin-free formaldehyde, and preservatives, which have been found safe and not contributing to undesirable effects in the organism to which the composition is administered.

### Pharmaceutical Purposes

The administration of the composition may be for either a “prophylactic” or “therapeutic” purpose. When provided prophylactically, the compositions of the disclosure are provided before any symptom or clinical sign of an infection becomes manifest. The prophylactic administration of the composition serves to prevent or attenuate any subsequent infection. The prophylactic administration of the composition serves to prevent or attenuate one or more symptoms or clinical signs associated with the disease resulting from the infection.

When provided therapeutically, a composition is provided upon the detection of a symptom or clinical sign of actual infection. The therapeutic administration serves to attenuate any actual or further infection.

Thus, a composition of the present disclosure may be provided either before the onset of infection (so as to prevent or attenuate an anticipated infection) or after the initiation of an actual infection.

A composition is said to be “pharmacologically acceptable” if its administration can be tolerated by a recipient mammal. Such an agent is said to be administered in a “therapeutically effective amount” if the amount administered is physiologically significant. A composition of the present disclosure is physiologically significant if its presence results in a detectable change in the physiology of a recipient patient. In particular, a “therapeutically effective amount” refers to an amount effective, at dosages and for periods of time necessary, to achieve the desired therapeutic result. A therapeutically effective amount is also one in which any toxic or detrimental effects of the agent(s) are outweighed by the therapeutically beneficial effects.

The “protection” provided need not be absolute, i.e., the influenza infection need not be totally prevented or eradicated, if there is a statistically significant improvement compared with a control population or set of mammals. Protection may be limited to mitigating the severity or rapidity of onset of symptoms or clinical signs of the influenza virus infection.

### Pharmaceutical Administration

A composition having at least one influenza virus of the present disclosure, and optionally one or more other isolated viruses, one or more isolated viral proteins disclosed herein, one or more isolated nucleic acid molecules encoding one or more viral proteins disclosed herein, or a combination thereof, may be administered by any means that achieve the intended purposes.

For example, administration of such a composition may be by various parenteral routes such as subcutaneous, intravenous, intradermal, intramuscular, intraperitoneal, intranasal, oral or transdermal routes. Parenteral administration can be accomplished by bolus injection or by gradual perfusion over time.

A typical regimen for preventing, suppressing, or treating an influenza virus related pathology, comprises administration of an effective amount of a vaccine composition as described herein, administered as a single treatment, or repeated as enhancing or booster dosages, over a period up to and including between one week and about 24 months, or any range or value therein.

According to the present disclosure, an “effective amount” of a composition is one that is sufficient to achieve a desired effect. It is understood that the effective dosage may be dependent upon the species, age, sex, health, and weight of the recipient, kind of concurrent treatment, if any, frequency of treatment, and the nature of the effect wanted. The ranges of effective doses provided below are not intended to limit the invention and represent dose ranges.

The dosage of a virus vaccine for an animal such as a mammalian adult organism may be from about 10²-10¹⁵, e.g., 10³-10¹², plaque forming units (PFU)/kg, or any range or value therein. The dose of inactivated vaccine may range from about 0.1 to 1000, e.g., 10 to 100 μg, such as about 15 μg, of HA protein. However, the dosage should be a safe and effective amount as determined by conventional methods, using existing vaccines as a starting point.

The dosage of immunoreactive HA in each dose of virus vaccine may be standardized to contain a suitable amount, e.g., 30 to 100 μg or any range or value therein, such as about 15 μg, or the amount recommended by government agencies or recognized professional organizations. The quantity of NA can also be standardized, however, this glycoprotein may be labile during purification and storage.

The dosage of immunoreactive HA in each dose of replicated virus vaccine can be standardized to contain a suitable amount, e.g., 1-50 μg or any range or value therein, or the amount recommended by the U.S. Public Health Service (PHS), which is usually 15 μg, per component for older children more than 3 years of age, and 7.5 μg per component for older children <3 years of age. The quantity of NA can also be standardized, however, this glycoprotein can be labile during the processor purification and storage (Kendal et al., 1980; Kerr et al., 1975). Each 0.5-ml dose of vaccine may contain approximately 1-50 billion virus particles, e.g., 10 billion particles.

### Gene Delivery Vectors

Gene delivery vectors include, for example, viral vectors, non-viral vectors, liposomes and other lipid-containing complexes, such as lipoplexes (DNA and cationic lipids), polyplexes, e.g., DNA complexed with cationic polymers such as polyethylene glycol, nanoparticles, e.g., magnetic inorganic nanoparticles that bind or are functionalized to bind DNA such as Fe₃O₄ or MnO₂ nanoparticles, nanoparticles, microparticles, e.g., formed of polylactide polygalactide reagents, nanotubes, e.g., silica nanotubes, and other macromolecular complexes capable of mediating delivery of a gene to a host cell. Vectors can also comprise other components or functionalities that further modulate gene delivery and/or gene expression, or that otherwise provide beneficial properties to the targeted cells. Such other components include, for example, components that influence binding or targeting to cells (including components that mediate cell-type or tissue-specific binding); components that influence uptake of the vector by the cell; components that influence localization of the transferred gene within the cell after uptake (such as agents mediating nuclear localization); and components that influence expression of the gene. Such components also might include markers, such as detectable and/or selectable markers that can be used to detect or select for cells that have taken up and are expressing the nucleic acid delivered by the vector. A large variety of such vectors are known in the art and are generally available.

Gene delivery vectors within the scope of the disclosure include, but are not limited to, isolated nucleic acid, e.g., plasmid-based vectors which may be extrachromosomally maintained and isolated RNA, and viral vectors, e.g., recombinant infleunza virus, adenovirus, retrovirus, lentivirus, herpesvirus, poxvirus, papilloma virus, or adeno-associated virus, including viral and non-viral vectors which are present in liposomes, e.g., neutral or cationic liposomes, such as DOSPA/DOPE, DOGS/DOPE or DMRIE/DOPE liposomes, and/or associated with other molecules such as DNA-anti-DNA antibody-cationic lipid (DOTMA/DOPE) complexes Exemplary viral gene delivery vectors are described below. Gene delivery vectors may be administered via any route including, but not limited to, intracranial, intrathecal, intramuscular, buccal, rectal, intravenous or intracoronary administration, and transfer to cells may be enhanced using electroporation and/or iontophoresis, and/or scaffolding such as extracellular matrix or hydrogels, e.g., a hydrogel patch. In one embodiment, a permeation enhancer is not employed to enhance indirect delivery to the CNS.

### Polypeptides

The polypeptides can be synthesized in vitro, e.g., by the solid phase peptide synthetic method or by recombinant DNA approaches (see above). The solid phase peptide synthetic method is an established and widely used method. These polypeptides can be further purified by fractionation on immunoaffinity or ion-exchange columns; ethanol precipitation; reverse phase HPLC; chromatography on silica or on an anion-exchange resin such as DEAE; chromatofocusing; SDS-PAGE; ammonium sulfate precipitation; gel filtration using, for example, Sephadex G-75; or ligand affinity chromatography.

Once isolated and characterized, chemically modified derivatives of a givenpolypeptide, can be readily prepared. For example, amides of the polypeptide of the present disclosure may also be prepared by techniques well known in the art for converting a carboxylic acid group or precursor, to an amide. One method for amide formation at the C-terminal carboxyl group is to cleave the polypeptide from a solid support with an appropriate amine, or to cleave in the presence of an alcohol, yielding an ester, followed by aminolysis with the desired amine.

Salts of carboxyl groups of a polypeptide may be prepared in the usual manner by contacting the polypeptide with one or more equivalents of a desired base such as, for example, a metallic hydroxide base, e.g., sodium hydroxide; a metal carbonate or bicarbonate base such as, for example, sodium carbonate or sodium bicarbonate; or an amine base such as, for example, triethylamine, triethanolamine, and the like.

N-acyl derivatives of an amino group of the polypeptide may be prepared by utilizing an N-acyl protected amino acid for the final condensation, or by acylating a protected or unprotected peptide, polypeptide, or fusion thereof. O-acyl derivatives may be prepared, for example, by acylation of a free hydroxy polypeptide or polypeptide resin. Either acylation may be carried out using standard acylating reagents such as acyl halides, anhydrides, acyl imidazoles, and the like. Both N- and O-acylation may be carried out together, if desired.

Formyl-methionine, pyroglutamine and trimethyl-alanine may be substituted at the N-terminal residue of the polypeptide. Other amino-terminal modifications include aminooxypentane modifications.

In one embodiment, a polypeptide has substantial identity, e.g., at least 80% or more, e.g., 85%, 87%, 90%, 92%, 95%, 97%, 98%, 99% and up to 100%, amino acid sequence identity to one of SEQ ID NOs. 1-44, or a portion thereof.

Substitutions may include substitutions which utilize the D rather than L form, as well as other well known amino acid analogs, e.g., unnatural amino acids such as α, α-disubstituted amino acids, N-alkyl amino acids, lactic acid, and the like. These analogs include phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine, ornithine, citruline, α-methyl-alanine, para-benzoyl-phenylalanine, phenylglycine, propargylglycine, sarcosine, ε-N,N,N-trimethyllysine, ε-N-acetyllysine, N-acetylserine, N-formylmethionine, 3-methylhistidine, 5-hydroxylysine, ω-N-methylarginine, and other similar amino acids and imino acids and tert-butylglycine.

Conservative amino acid substitutions may be employed—that is, for example, aspartic-glutamic as acidic amino acids; lysine/arginine/histidine as polar basic amino acids; leucine/isoleucine/methionine/valine/alanine/proline/glycine non-polar or hydrophobic amino acids; serine/threonine as polar or hydrophilic amino acids. Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine, and a group of amino acids having sulfur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting peptide, polypeptide or fusion polypeptide. Whether an amino acid change results in a functional peptide, polypeptide or fusion polypeptide can readily be determined by assaying the specific activity of the peptide, polypeptide or fusion polypeptide.

Amino acid substitutions falling within the scope of the disclosure, are, in general, accomplished by selecting substitutions that do not differ significantly in their effect on maintaining (a) the structure of the peptide backbone in the area of the substitution, (b) the charge or hydrophobicity of the molecule at the target site, or (c) the bulk of the side chain. Naturally occurring residues are divided into groups based on common side-chain properties:
(1) hydrophobic: norleucine, met, ala, val, leu, ile;
(2) neutral hydrophilic: cys, ser, thr;
(3) acidic: asp, glu;
(4) basic: asn, gln, his, lys, arg;
(5) residues that influence chain orientation: gly, pro; and
(6) aromatic; trp, tyr, phe.

The disclosure also envisions a peptide, polypeptide or fusion polypeptide with non-conservative substitutions. Non-conservative substitutions entail exchanging a member of one of the classes described above for another.

Acid addition salts of the peptide, polypeptide or fusion polypeptide or of amino residues of the peptide, polypeptide or fusion polypeptide may be prepared by contacting the polypeptide or amine with one or more equivalents of the desired inorganic or organic acid, such as, for example, hydrochloric acid. Esters of carboxyl groups of the polypeptides may also be prepared by any of the usual methods known in the art.

### Exemplary Compositions and Routes of Delivery for the Compositions

Any route of administration may be employed so long as that route and the amount administered are prophylactically or therapeutically useful.

In vivo administration of virus, e.g., delivered in a viral vector such as an influenza virus, nucleic acid encoding a gene product or protein (polypeptide), and compositions containing them, or delivery systems such as nanoparticles or liposomes having the vector or polypeptide, can be accomplished by any suitable method and technique presently or prospectively known to those skilled in the art. The subject polynucleotides or polypeptides can be formulated in a physiologically- or pharmaceutically-acceptable form and administered by any suitable route known in the art including, for example, oral, nasal, rectal, transdermal, vaginal, and parenteral routes of administration. As used herein, the term parenteral includes subcutaneous, intradermal, intravenous, intramuscular, intraperitoneal, and intracisternal administration, such as by injection.

Administration of the compositions can be a single administration, or at continuous or distinct intervals as can be readily determined by a person skilled in the art.

The nucleic acid sand polypeptide compositions can be administered utilizing liposome and nano-technology, slow release capsules, implantable pumps, and biodegradable containers, and orally or intestinally administered intact plant cells expressing the therapeutic product. These delivery methods can, advantageously, provide a uniform dosage over an extended period of time.

Suitable dose ranges for are generally about 10³ to 10¹⁵ infectious units of viral vector per microliter delivered in 1 to 3000 microliters of single injection volume. For instance, viral genomes or infectious units of vector per micro liter would generally contain about 10⁴, 10⁵, 10⁶, 10⁷, 10⁸, 10⁹, 10¹⁰, 10¹¹, 10¹², 10¹³, 10¹⁴, 10¹⁵, 10¹⁶, or 10¹⁷ viral genomes or infectious units of viral vector delivered in about 10, 50, 100, 200, 500, 1000, or 2000 microliters.

In one embodiment, the amount of a vector to be administered, e.g., to a human, is about 1 ng to 1 g, e.g., about 1 ng to 100 ng, 100 ng to 250 ng, 250 ng to 750 ng, 750 ng to 1000 ng, 1 mg to 100 mg, 100 mg to 250 mg, 250 mg to 750 mg, or 750 mg to 1000 mg.

In one embodiment, the amount of virus to be administered, e.g., to a human, is about 10⁶ to 10¹⁵ viral genomes, focus forming units (FFU) or infectious units (IU), e.g., about 10⁶ to 10⁷, 10⁷ to 10⁸, 10⁸ to 10⁹, 10⁹ to 10¹⁰, 10¹⁰ to 10¹¹, 10¹¹ to 10¹², 10¹² to 10¹³, 10¹³ to 10¹⁴, or 10¹⁴ to 10¹⁵, viral genomes, FFU or IU.

In one embodiment, the amount of a protein to be administered, e.g., to a human, is about 1 mg to 1 g. e.g., 1 mg to 100 mg, 100 mg to 250 mg, 250 mg to 750 mg, or 750 mg to 1000 mg.

It should be understood that the aforementioned dosage is merely an exemplary dosage and those of skill in the art will understand that this dosage may be varied. Effective doses may be extrapolated from dose-responsive curves derived from in vitro or in vivo test systems.

In one embodiment, suitable dose ranges are generally about 10³ to 10¹⁵ infectious units of viral vector per microliter delivered in, for example, 1, 2, 5, 10, 25, 50, 75 or 100 or more milliliters, e.g., 1 to 10,000 milliliters or 0.5 to 15 milliliters, of single injection volume. For instance, viral genomes or infectious units of vector per microliter would generally contain about 10⁴, 10⁵, 10⁶, 10⁷, 10⁸, 10⁹, 10¹⁰, 10¹¹, 10¹², 10¹³, or 10¹⁴ viral genomes or infectious units (IU) of viral vector. In one embodiment, suitable dose ranges, generally about 10³ to 10¹⁵ infectious units of viral vector per microliter delivered in, for example, 1, 2, 5, 10, 25, 50, 75 or 100 or more milliliters, e.g., 1 to 10,000 milliliters or 0.5 to 15 milliliters. For instance, viral genomes or infectious units of vector per microliter would generally contain about 10⁴, 10⁵, 10⁶, 10⁷, 10⁸, 10⁹, 10¹⁰, 10¹¹, 10¹², 10¹³, 10¹⁴, 10¹⁵, 10¹⁶, or 10¹⁷ viral genomes or infectious units of viral vector, e.g., at least 1.2×10¹¹ genomes or infectious units, for instance at least 2×10¹¹ up to about 2×10¹² genomes or infectious units or about 1×10¹³ to about 5×10¹⁶ genomes or infectious units.

Administration of nucleic acid vectors, e.g., non-viral vectors, virus or polypeptides in accordance with the present disclosure can be achieved by direct injection of the composition or by the use of infusion pumps. For injection, the composition can be formulated in liquid solutions, e.g., in physiologically compatible buffers such as Hank's solution, Ringer's solution or phosphate buffer. In addition, the enzyme may be formulated in solid form and re-dissolved or suspended immediately prior to use. Lyophilized forms are also included. The injection can be, for example, in the form of a bolus injection or continuous infusion (e.g., using infusion pumps) of the enzyme.

In one embodiment, the vector, polypeptide or virus may be administered by any route including parenterally. In one embodiment, the vector, polypeptide or virus may be administered by subcutaneous, intramuscular, or intravenous injection, orally, intrathecally, or intracranially, or by sustained release, e.g., using a subcutaneous implant. The agent(s) may be dissolved or dispersed in a liquid carrier vehicle. For parenteral administration, the active material may be suitably admixed with an acceptable vehicle, e.g., of the vegetable oil variety such as peanut oil, cottonseed oil and the like. Other parenteral vehicles such as organic compositions using solketal, glycerol, formal, and aqueous parenteral formulations may also be used. For parenteral application by injection, the agent(s) may comprise an aqueous solution of a water soluble pharmaceutically acceptable salt of the active acids according to the invention, desirably in a concentration of 0.01-10%, and optionally also a stabilizing agent and/or buffer substances in aqueous solution. Dosage units of the solution may advantageously be enclosed in ampules.

The vector, polypeptide or virus may be in the form of an injectable unit dose. Examples of carriers or diluents usable for preparing such injectable doses include diluents such as water, ethyl alcohol, macrogol, propylene glycol, ethoxylated isostearyl alcohol, polyoxyisostearyl alcohol and polyoxyethylene sorbitan fatty acid esters, pH adjusting agents or buffers such as sodium citrate, sodium acetate and sodium phosphate, stabilizers such as sodium pyrosulfite, EDTA, thioglycolic acid and thiolactic acid, isotonic agents such as sodium chloride and glucose, local anesthetics such as procaine hydrochloride and lidocaine hydrochloride. Furthermore, usual solubilizing agents and analgesics may be added. Injections can be prepared by adding such carriers to the enzyme or other active, following procedures well known to those of skill in the art. A thorough discussion of pharmaceutically acceptable excipients is available in REMINGTON'S PHARMACEUTICAL SCIENCES (Mack Pub. Co., N.J. 1991). The pharmaceutically acceptable formulations can easily be suspended in aqueous vehicles and introduced through conventional hypodermic needles or using infusion pumps. Prior to introduction, the formulations can be sterilized with, preferably, gamma radiation or electron beam sterilization.

When the vector, polypeptide or virus is administered in the form of a subcutaneous implant, the compound is suspended or dissolved in a slowly dispersed material known to those skilled in the art, or administered in a device which slowly releases the active material through the use of a constant driving force such as an osmotic pump. In such cases, administration over an extended period of time is possible.

The dosage at which the vector, polypeptide or virus is administered may vary within a wide range and will depend on various factors such as the severity of the disease, the age of the patient, etc., and may have to be individually adjusted. Compositions described herein may be employed in combination with another medicament. The compositions can appear in conventional forms, for example, aerosols, solutions, suspensions, or topical applications, or in lyophilized form.

Typical compositions include the vector, polypeptide or virus and a pharmaceutically acceptable excipient which can be a carrier or a diluent. For example, the vector, polypeptide or virus may be mixed with a carrier, or diluted by a carrier, or enclosed within a carrier. When the vector, polypeptide or virus is mixed with a carrier, or when the carrier serves as a diluent, it can be solid, semi-solid, or liquid material that acts as a vehicle, excipient, or medium for the active agent. Some examples of suitable carriers are water, salt solutions, alcohols, polyethylene glycols, polyhydroxyethoxylated castor oil, peanut oil, olive oil, gelatin, lactose, terra alba, sucrose, dextrin, magnesium carbonate, sugar, cyclodextrin, amylose, magnesium stearate, talc, gelatin, agar, pectin, acacia, stearic acid or lower alkyl ethers of cellulose, silicic acid, fatty acids, fatty acid amines, fatty acid monoglycerides and diglycerides, pentaerythritol acid fatty esters, polyoxyethylene, hydroxymethylcellulose and polyvinylpyrrolidone. Similarly, the carrier or diluent can include any sustained release material known in the art, such as glyceryl monostearate or glyceryl distearate, alone or mixed with a wax.

The formulations can be mixed with auxiliary agents which do not deleteriously react with the vector, polypeptide or virus. Such additives can include wetting agents, emulsifying and suspending agents, salt for influencing osmotic pressure, buffers and/or coloring substances preserving agents, sweetening agents or flavoring agents. The compositions can also be sterilized if desired.

If a liquid carrier is used, the preparation can be in the form of a liquid such as an aqueous liquid suspension or solution. Acceptable solvents or vehicles include sterilized water, Ringer's solution, or an isotonic aqueous saline solution.

The vector, polypeptide or virus may be provided as a powder suitable for reconstitution with an appropriate solution as described above. Examples of these include, but are not limited to, freeze dried, rotary dried or spray dried powders, amorphous powders, granules, precipitates, or particulates. The composition can optionally contain stabilizers, pH modifiers, surfactants, bioavailability modifiers and combinations of these. A unit dosage form can be in individual containers or in multi-dose containers.

Compositions contemplated by the present disclosure may include, for example, micelles or liposomes, or some other encapsulated form, such as nanoparticles, or can be administered in an extended release form to provide a prolonged storage and/or delivery effect, e.g., using biodegradable polymers, e.g., polylactide-polyglycolide. Examples of other biodegradable polymers include poly(orthoesters) and poly(anhydrides).

Polymeric nanoparticles, e.g., comprised of a hydrophobic core of polylactic acid (PLA) and a hydrophilic shell of methoxy-poly(ethylene glycol) (MPEG), may have improved solubility and targeting to the CNS. Regional differences in targeting between the microemulsion and nanoparticle formulations may be due to differences in particle size.

Liposomes are very simple structures consisting of one or more lipid bilayers of amphiphilic lipids, i.e., phospholipids or cholesterol. The lipophilic moiety of the bilayers is turned towards each other and creates an inner hydrophobic environment in the membrane. Liposomes are suitable drug carriers for some lipophilic drugs which can be associated with the non-polar parts of lipid bilayers if they fit in size and geometry. The size of liposomes varies from 20 nm to few μm.

Mixed micelles are efficient detergent structures which are composed of bile salts, phospholipids, tri, di- and monoglycerides, fatty acids, free cholesterol and fat soluble micronutrients. As long-chain phospholipids are known to form bilayers when dispersed in water, the preferred phase of short chain analogues is the spherical micellar phase. A micellar solution is a thermodynamically stable system formed spontaneously in water and organic solvents. The interaction between micelles and hydrophobic/lipophilic drugs leads to the formation of mixed micelles (MM), often called swallen micelles, too. In the human body, they incorporate hydrophobic compounds with low aqueous solubility and act as a reservoir for products of digestion, e.g. monoglycerides.

Lipid microparticles includes lipid nano- and microspheres. Microspheres are generally defined as small spherical particles made of any material which are sized from about 0.2 to 100 μm. Smaller spheres below 200 nm are usually called nanospheres. Lipid microspheres are homogeneous oil/water microemulsions similar to commercially available fat emulsions, and are prepared by an intensive sonication procedure or high pressure emulsifying methods (grinding methods). The natural surfactant lecithin lowers the surface tension of the liquid, thus acting as an emulsifier to form a stable emulsion. The structure and composition of lipid nanospheres is similar to those of lipid microspheres, but with a smaller diameter.

Polymeric nanoparticles serve as carriers for a broad variety of ingredients. The active components may be either dissolved in the polymetric matrix or entrapped or adsorbed onto the particle surface. Polymers suitable for the preparation of organic nanoparticles include cellulose derivatives and polyesters such as poly(lactic acid), poly(glycolic acid) and their copolymer. Due to their small size, their large surface area/volume ratio and the possibility of functionalization of the interface, polymeric nanoparticles are ideal carrier and release systems. If the particle size is below 50 nm, they are no longer recognized as particles by many biological and also synthetic barrier layers, but act similar to molecularly disperse systems.

Thus, the composition can be formulated to provide quick, sustained, controlled, or delayed release, or any combination thereof, of the active agent after administration to the individual by employing procedures well known in the art. In one embodiment, the enzyme is in an isotonic or hypotonic solution. In one embodiment, for enzymes that are not water soluble, a lipid based delivery vehicle may be employed, e.g., a microemulsion such as that described in WO 2008/049588, the disclosure of which is incorporated by reference herein, or liposomes.

In one embodiment, the preparation can contain an agent, dissolved or suspended in a liquid carrier, such as an aqueous carrier, for aerosol application. The carrier can contain additives such as solubilizing agents, e.g., propylene glycol, surfactants, absorption enhancers such as lecithin (phosphatidylcholine) or cyclodextrin, or preservatives such as parabens.

### Exemplary Polymer Delivery Vehicles

In one embodiment, the delivery vehicle is a naturally occurring polymer, e.g., formed of materials including but not limited to albumin, collagen, fibrin, alginate, extracellular matrix (ECM), e.g., xenogeneic ECM, hyaluronan (hyaluronic acid), chitosan, gelatin, keratin, potato starch hydrolyzed for use in electrophoresis, or agar-agar (agarose). In one embodiment, the delivery vehicle comprises a hydrogel. In one embodiment, the composition comprises a naturally occurring polymer. Table 1 provides exemplary materials for delivery vehicles that are formed of naturally occurring polymers and materials for particles.

TABLE 1

| Particle class | Materials |
|---|---|
| Natural materials or | Chitosan |
| derivatives | Dextran |
| | Gelatine |
| | Albumin |
| | Alginates |
| | Liposomes |
| | Starch |
| Polymer carriers | Polylactic acid |
| | Poly(cyano)acrylates |
| | Polyethyleneimine |
| | Block copolymers |
| | Polycaprolactone |

An exemplary polycaprolactone is methoxy poly(ethylene glycol)/poly(epsilon caprolactone). An exemplary poly lactic acid is poly(D,L-lactic-co-glycolic)acid (PLGA).

Some examples of materials for particle formation include but are not limited to agar acrylic polymers, polyacrylic acid, poly acryl methacrylate, gelatin, poly(lactic acid), pectin(poly glycolic acid), cellulose derivatives, cellulose acetate phthalate, nitrate, ethyl cellulose, hydroxyl ethyl cellulose, hydroxypropylcellulose, hydroxyl propyl methyl cellulose, hydroxypropylmethylcellulose phthalate, methyl cellulose, sodium carboxymethylcellulose, poly(ortho esters), polyurethanes, poly(ethylene glycol), poly(ethylene vinyl acetate), polydimethylsiloxane, poly(vinyl acetate phthalate), polyvinyl alcohol, polyvinyl pyrrollidone, and shellac. Soluble starch and its derivatives for particle preparation include amylodextrin, amylopectin and carboxy methyl starch.

In one embodiment, the polymers in the nanoparticles or microparticles are biodegradable. Examples of biodegradable polymers useful in particles preparation include synthetic polymers, e.g., polyesters, poly(ortho esters), polyanhydrides, or polyphosphazenes; natural polymers including proteins (e.g., collagen, gelatin, and albumin), or polysaccharides (e.g., starch, dextran, hyaluronic acid, and chitosan). For instance, a biocompatible polymer includes poly (lactic) acid (PLA), poly (glycolic acid) (PLGA). Natural polymers that may be employed in particles (or as the delivery vehicle) include but are not limited to albumin, chitin, starch, collagen, chitosan, dextrin, gelatin, hyaluronic acid, dextran, fibrinogen, alginic acid, casein, fibrin, and polyanhydrides.

In one embodiment, the delivery vehicle is a hydrogel Hydrogels can be classified as those with chemically crosslinked networks having permanent junctions or those with physical networks having transient junctions arising from polymer chain entanglements or physical interactions, e.g., ionic interactions, hydrogen bonds or hydrophobic interactions. Natural materials useful in hydrogels include natural polymers, which are biocompatible, biodegradable, support cellular activities, and include proteins like fibrin, collagen and gelatin, and polysaccharides like starch, alginate and agarose.

In one embodiment, the delivery vehicle comprises inorganic nanoparticles, e.g., calcium phosphate or silica particles; polymers including but not limited to poly(lactic-co-glycolic acid) (PLGA), polylactic acid (PLA), linear and/or branched PEI with differing molecular weights (e.g., 2, 22 and 25 kDa), dendrimers such as polyamidoamine (PAMAM) and polymethoacrylates; lipids including but not limited to cationic liposomes, cationic emulsions, DOTAP, DOTMA, DMRIE, DOSPA, distearoylphosphatidylcholine (DSPC), DOPE, or DC-cholesterol; peptide based vectors including but not limited to Poly-L-lysine or protamine; or poly(β-amino ester), chitosan, PEI-polyethylene glycol, PEI-mannose-dextrose, DOTAP-cholesterol or RNAiMAX.

In one embodiment, the delivery vehicle is a glycopolymer-based delivery vehicle, poly(glycoamidoamine)s (PGAAs), that have the ability to complex with various polynucleotide types and form nanoparticles. These materials are created by polymerizing the methylester or lactone derivatives of various carbohydrates (D-glucarate (D), meso-galactarate (G), D-mannarate (M), and L-tartarate (T)) with a series of oligoethyleneamine monomers (containing between 1-4 ethylenamines. A subset composed of these carbohydrates and four ethyleneamines in the polymer repeat units yielded exceptional delivery efficiency.

In one embodiment, the delivery vehicle comprises polyethyleneimine (PEI), Polyamidoamine (PAMAM), PEI-PEG, PEI-PEG-mannose, dextran-PEI, OVA conjugate, PLGA microparticles, or PLGA microparticles coated with PAMAM.

In one embodiment, the delivery vehicle comprises a cationic lipid, e.g., N-[1-(2,3-dioleoyloxy)propel]-N,N,N-trimethylammonium (DOTMA), 2,3-dioleyloxy-N-[2-spermine carboxamide] ethyl-N,N-dimethyl-1-propanammonium trifluoracetate (DOSPA, Lipofectamine); 1,2-dioleoyl-3-trimethylammonium-propane (DOTAP); N-[1-(2,3-dimyristloxy) propyl]: N,N-dimethyl-N-(2-hydroxyethyl) ammonium bromide (DMRIE), 3-β-[N—(N,N′-dimethylaminoethane) carbamoyl] cholesterol (DC-Chol); dioctadecyl amidoglyceryl spermine (DOGS, Transfectam); or imethyldioctadeclyammonium bromide (DDAB). The positively charged hydrophilic head group of cationic lipids usually consists of monoamine such as tertiary and quaternary amines, polyamine, amidinium, or guanidinium group. A series of pyridinium lipids have been developed. In addition to pyridinium cationic lipids, other types of heterocyclic head group include imidazole, piperizine and amino acid. The main function of cationic head groups is to condense negatively charged nucleic acids by means of electrostatic interaction to slightly positively charged nanoparticles, leading to enhanced cellular uptake and endosomal escape.

Lipids having two linear fatty acid chains, such as DOTMA, DOTAP and SAINT-2, or DODAC, may be employed as a delivery vehicle, as well as tetraalkyl lipid chain surfactant, the dimer of N,N-dioleyl-N,N-dimethylammonium chloride (DODAC). All the trans-orientated lipids regardless of their hydrophobic chain lengths (C_{16:1}, C_{18:1} and C_{20:1}) appear to enhance the transfection efficiency compared with their cis-orientated counterparts.

The structures of cationic polymers useful as a delivery vehicle include but are not limited to linear polymers such as chitosan and linear poly(ethyleneimine), branched polymers such as branch poly(ethyleneimine) (PEI), circle-like polymers such as cyclodextrin, network (crosslinked) type polymers such as crosslinked poly(amino acid) (PAA), and dendrimers. Dendrimers consist of a central core molecule, from which several highly branched arms ‘grow’ to form a tree-like structure with a manner of symmetry or asymmetry. Examples of dendrimers include polyamidoamine (PAMAM) and polypropylenimine (PPI) dendrimers.

DOPE and cholesterol are commonly used neutral co-lipids for preparing cationic liposomes. Branched PEI-cholesterol water-soluble lipopolymer conjugates self-assemble into cationic micelles. Pluronic (poloxamer), a non-ionic polymer and SP1017, which is the combination of Pluronics L61 and F127, may also be used.

In one embodiment, PLGA particles are employed to increase the encapsulation frequency although complex formation with PLL may also increase the encapsulation efficiency. Other cationic materials, for example, PEI, DOTMA, DC-Chol, or CTAB, may be used to make nanospheres.

In one embodiment, the particles comprise at least one polymeric material. In one embodiment, the polymeric material is biodegradable. In one embodiment, polymeric materials include: silk, elastin, chitin, chitosan, poly(a-hydroxy acids), poly(anhydrides), and poly(orthoesters). In one embodiment, the biodegradable microparticle may comprise polyethylene glycol, poly(lactic acid), poly(glycolic acid), copolymers of lactic and glycolic acid, copolymers of lactic and glycolic acid with polyethylene glycol, poly(E-caprolactone), poly(3-hydroxybutyrate), poly(p-dioxanone), polypropylene fumarate, poly(orthoesters), polyol/diketene acetals addition polymers, poly(sebacic anhydride) (PSA), poly(carboxybiscarboxyphenoxyphenoxy hexone (PCPP) poly[bis (p-carboxypheonoxy) methane] (PCPM), copolymers of SA, CPP and CPM, poly(amino acids), poly(pseudo amino acids), polyphosphazenes, derivatives of poly[(dichloro)phosphazenes] and poly[(organo) phosphazenes], poly-hydroxybutyric acid, or S-caproic acid, polylactide-co-glycolide, polylactic acid, and polyethylene glycol. Polyesters may be employed.

### Exemplary Formulations

In certain embodiments, a formulation comprises a biocompatible polymer selected from the group consisting of polyamides, polycarbonates, polyalkylenes, polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho)esters, poly(butic acid), poly(valeric acid), poly(lactide-co-caprolactone), polysaccharides, proteins, polyhyaluronic acids, polycyanoacrylates, and blends, mixtures, or copolymers thereof.

The composition can be administered in or on a device that allows controlled or sustained release, such as a sponge, biocompatible meshwork, mechanical reservoir, or mechanical implant. Implants (see, e.g., U.S. Pat. No. 5,443,505), devices (see, e.g., U.S. Pat. No. 4,863,457), such as an implantable device, e.g., a mechanical reservoir or an implant or a device comprised of a polymeric composition, are particularly useful for administration of the inventive gene transfer vector. The composition also can be administered in the form of sustained-release formulations (see, e.g., U.S. Pat. No. 5,378,475) comprising, for example, gel foam, hyaluronic acid, gelatin, chondroitin sulfate, a polyphosphoester, such as bis-2-hydroxyethyl-terephthalate (BHET), and/or a polylactic-glycolic acid.

Vectors may conveniently be provided in the form of formulations suitable for administration, e.g., into the brain. A suitable administration format may best be determined by a medical practitioner for each patient individually, according to standard procedures. Suitable pharmaceutically acceptable carriers and their formulation are described in standard formulations treatises, e.g., Remington's Pharmaceuticals Sciences. By “pharmaceutically acceptable” it is meant a carrier, diluent, excipient, and/or salt that is compatible with the other ingredients of the formulation, and not deleterious to the recipient thereof.

Vectors may be formulated in solution at neutral pH, for example, about pH 6.5 to about pH 8.5, or from about pH 7 to 8, with an excipient to bring the solution to about isotonicity, for example, 4.5% mannitol or 0.9% sodium chloride, pH buffered with art-known buffer solutions, such as sodium phosphate, that are generally regarded as safe, together with an accepted preservative such as metacresol 0.1% to 0.75%, or from 0.15% to 0.4% metacresol. Obtaining a desired isotonicity can be accomplished using sodium chloride or other pharmaceutically acceptable agents such as dextrose, boric acid, sodium tartrate, propylene glycol, polyols (such as mannitol and sorbitol), or other inorganic or organic solutes. Sodium chloride is useful for buffers containing sodium ions. If desired, solutions of the above compositions can also be prepared to enhance shelf life and stability. Therapeutically useful compositions of the disclosure can be prepared by mixing the ingredients following generally accepted procedures. For example, the selected components can be mixed to produce a concentrated mixture which may then be adjusted to the final concentration and viscosity by the addition of water and/or a buffer to control pH or an additional solute to control tonicity.

The vectors can be provided in a dosage form containing an amount of a vector effective in one or multiple doses. For viral vectors, the effective dose may be in the range of at least about 10⁷ viral particles, e.g., about 10⁹ viral particles, or about 10¹¹ viral particles. The number of viral particles added may be up to 10¹⁴. For example, when a viral expression vector is employed, about 10⁸ to about 10⁶⁰ gc of viral vector can be administered as nucleic acid or as a packaged virion. In some embodiments, about 10⁹ to about 10¹⁵ copies of viral vector, e.g., per 0.5 to 10 mL, can be administered as nucleic acid or as a packaged virion. Alternatively, the nucleic acids or vectors, can be administered in dosages of at least about 0.0001 mg/kg to about 1 mg/kg, of at least about 0.001 mg/kg to about 0.5 mg/kg, at least about 0.01 mg/kg to about 0.25 mg/kg or at least about 0.01 mg/kg to about 0.25 mg/kg of body weight, although other dosages may provide beneficial results. As noted, the exact dose to be administered is determined by the attending clinician, but may be in 1 mL phosphate buffered saline. For delivery of plasmid DNA alone, or plasmid DNA in a complex with other macromolecules, the amount of DNA to be administered will be an amount which results in a beneficial effect to the recipient. For example, from 0.0001 to 1 mg or more, e.g., up to 1 g, in individual or divided doses, e.g., from 0.001 to 0.5 mg, or 0.01 to 0.1 mg, of DNA can be administered.

For example, when a viral expression vector is employed, about 10⁸ to about 10⁶⁰ gc of viral vector can be administered as nucleic acid or as a packaged virion. In some embodiments, about 10⁹ to about 10¹⁵ copies of viral vector, e.g., per 0.5 to 10 mL, can be administered as nucleic acid or as a packaged virion. Alternatively, the nucleic acids or vectors, can be administered in dosages of at least about 0.0001 mg/kg to about 1 mg/kg, of at least about 0.001 mg/kg to about 0.5 mg/kg, at least about 0.01 mg/kg to about 0.25 mg/kg or at least about 0.01 mg/kg to about 0.25 mg/kg of body weight, although other dosages may provide beneficial results.

Pharmaceutical formulations can be prepared by procedures known in the art using well known and readily available ingredients. For example, the active agent can be formulated with common excipients, diluents, or carriers, and formed into tablets, capsules, suspensions, powders, and the like. The vectors of the disclosure can also be formulated as elixirs or solutions appropriate for parenteral administration, for instance, by intramuscular, subcutaneous or intravenous routes.

The pharmaceutical formulations can also take the form of an aqueous or anhydrous solution, e.g., a lyophilized formulation, or dispersion, or alternatively the form of an emulsion or suspension.

In one embodiment, the active agent may be formulated for administration, e.g., by injection, for example, bolus injection or continuous infusion via a catheter, and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

These formulations can contain pharmaceutically acceptable vehicles and adjuvants which are well known in the prior art. It is possible, for example, to prepare solutions using one or more organic solvent(s) that is/are acceptable from the physiological standpoint.

For administration to the upper (nasal) or lower respiratory tract by inhalation, the vector is conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, the composition may take the form of a dry powder, for example, a powder mix of the therapeutic agent and a suitable powder base such as lactose or starch. The powder composition may be presented in unit dosage form in, for example, capsules or cartridges, or, e.g., gelatine or blister packs from which the powder may be administered with the aid of an inhalator, insufflator or a metered-dose inhaler.

For intra-nasal administration, the vector may be administered via nose drops, a liquid spray, such as via a plastic bottle atomizer or metered-dose inhaler. Typical of atomizers are the Mistometer (Wintrop) and the Medihaler (Riker).

The formulations and compositions described herein may also contain other ingredients such as antimicrobial agents or preservatives.

### Subjects

The subject may be any animal, including a human and non-human animals. Non-human animals includes all vertebrates, e.g., mammals and non-mammals, such as non-human primates, sheep, dogs, cats, cows, horses, chickens, amphibians, and reptiles, although mammals are preferred, such as non-human primates, sheep, dogs, cats, cows and horses. The subject may also be livestock such as, cattle, swine, sheep, poultry, and horses, or pets, such as dogs and cats.

Subjects include human subjects suffering from or at risk for oxidative damage. The subject is generally diagnosed with the condition of the subject invention by skilled artisans, such as a medical practitioner.

The methods described herein can be employed for subjects of any species, gender, age, ethnic population, or genotype. Accordingly, the term subject includes males and females, and it includes elderly, elderly-to-adult transition age subjects adults, adult-to-pre-adult transition age subjects, and pre-adults, including adolescents, children, and infants.

Examples of human ethnic populations include Caucasians, Asians, Hispanics, Africans, African Americans, Native Americans, Semites, and Pacific Islanders. The methods may be more appropriate for some ethnic populations such as Caucasians, especially northern European populations, as well as Asian populations.

The term subject also includes subjects of any genotype or phenotype as long as they are in need of the invention, as described above. In addition, the subject can have the genotype or phenotype for any hair color, eye color, skin color or any combination thereof.

The term subject includes a subject of any body height, body weight, or any organ or body part size or shape.

The invention will be further described by the following non-limiting examples.

### Example 1: Materials and Methods

### Cells, Viruses, and Plasmids

Experiments were conducted on A549 (CCL-185), HEK 293T (CRL-3216), MDCK (CCCL-34), or MDCK-SIAT-TMPRSS2 cells (a kind gift from J. Bloom, citation). MDCK-SIAT-TMPRSS2 stably expressing PB2 were generated by retroviral gene delivery of a codon-optimized PB2 gene. Cells were maintained in Dulbecco's modified Eagle's medium (DMEM: Mediatech 10-013-CV) with 10% FBS and grown at 37° in 5% CO2.

pCDNA3-based vectors for WSN proteins and pBD bi-directional reverse genetics plasmids were previously described (Mehle and Doudna 2008). pBD-PB2-DelVG-V5 encodes a PB2 gene segment that expresses V5-tagged proteins only when translated from a DelVG. To achieve this, we followed prior work creating PB2-FLAG-143 by first duplicating the last 109 nt of the PB2 open reading frame and placing it downstream of the native PB2 ORF to create a contiguous packing sequence. The last 106 nt of the PB2 ORF was codon optimized to avoid direct repeats. Another copy of the last 330 nt of the PB2 ORF was inserted downstream of the native PB2 stop codon. This repeat encoded sequence for the V5 epitope tag in all three frames at the 3′ end. It was further modified to remove upstream stop codons in all three frames. In this way, native PB2 is produced under normal circumstances and can support viral replication, with V5-tagged proteins are only produced if deletions arise that create junctions in the 3′ end of the PB2 ORF. DelVGs that arose during passage of the PB2-DelVG-V5 virus were cloned by RT-PCR and inserted into pCDNA3 for expression in mammalian cells (see below). Select DelVG were also cloned into pBD for creation of clonal DPR viruses. All sequence were confirmed by sequencing.

All virus and virus-derived protein expression constructs are based on A/WSN/1933. Viruses were rescued using the pBD bi-directional reverse genetics system. PB2-DelVG-V5 virus was generated by replacing wild type PB2 with PB2-DelVG-V5 to detect translation of proteins from a DVG. Viruses were rescued by transfecting reverse genetics plasmids into 293T cells, culture were overlaid 24 h later with MDCK-SIAT-TRMPSS2 cells, and virus was recovered 48-72 h after that. Viruses were amplified on MDCK-SIAT1-TMPRSS2 cells and titered on MDCK cells by plaque assay.

Clonal DVG viruses were created by substituting the PB2 gene segment with a cloned version of a DVG. PB2 protein was provided in trans during transfection of the 293T cells. Virus was amplified and titered using MDCK-SIAT-TMPRSS2 stably expressing PB2.

WSN PB2-DelVG-V5 virus and DVG viruses were confirmed by RT-PCR, sequencing, and western blot.

Transfections were completed using TransIT X2 following the manufacturers' recommendation.

### Antibodies

PB2-V5 proteins were captured using V5 trap agarose beads. The following antibodies were used for western blot analysis: rabbit anti-PB1, rabbit anti-PB2, rabbit anti-V5, and mouse anti-V5-HRP.

### Cloning DPRs

RNA was extracted from the supernatant of MDCK cells infected with the PB2-DelVG-V5 virus using Trizol. RNA was reverse-transcribed with Superscript III (Invitrogen) using primers specific to the PB2-DelVG-V5 segment. cDNA was amplified via PCR using primers annealing to PB2 UTRs that would capture full-length PB2 and DVG. Products were cloned into pCDNA3 or pBD.

### Co-Immunoprecipitation

HEK 293T cells were transfected with plasmids expressing NP, PB2-DelVG-V5, PB2, PB1 and PA. Cells were lysed in colP buffer (50 mM Tris pH 7.4, 150 mM NaCl, 0.5% NP40 and 1× protease inhibitors) and clarified by centrifugation. Truncated PB2-V5 proteins were captured with V5 trap agarose beads (Chromotek) and washed extensively. Precipitating proteins were detected by western blot.

### Polymerase Activity Assay

Polymerase activity assays were performed by transfecting HEK 293T cells with plasmids expressing NP, PB2, PB1 and PA and pHH21-vNA luc expressing a model vRNA encoding firefly luciferase. A plasmid expression renilla luciferase was included as a transfection control. Where indicated, PB2-DelVG-V5 proteins were co-expressed to test their inhibitory potential. Cells were lysed 24 hours after transfection in passive lysis buffer (Promega) and firefly and renilla luciferase activity was measured. Results were normalized to the internal control renilla. Protein expression was assessed by western blot.

### Transfection-Infection

HEK 293T cells were seeded into a 6 well plate and transfected the next day with plasmids expressing PB2-DelVG-V5 or an empty pCDNA3 control. Cells were split 1:2 24 h after transfection. 24 h after that, cells were inoculated with the WSN nanoluciferase reporter virus PASTN at an MOI of 05. Supernatants were harvested 24 h post-infection. Viral titers were measured by infecting MDCK cells in a 96 well plate and measuring nanoluciferase activity 8 h later with the Nano-Glo Luciferase Assay System (Promega).

### RNA Sequencing

RNA was extracted using Trizol from viral stocks of WSN or three plaque-purified stock of PB2-DelVG-V5. Sequencing was performed. DVG were identified using a modified version of ViReMa. The parallel-coordinate maps were generated by adapting an example code (https://observablehq.com/@drimaria/parallel-coordinates-d3-v4) from the D3.js platform.

### Ribsome Profiling

Ribosome profiling experiments were conducted in Tran and Ledwith (2021). Data were reanalyzed paying attention to read depth at the termini of gene segments and to specifically identify chimeric ribosome protected fragments (RPFs) spanning the deletion junction in the DVG. A modified version of ViReMa was used to map these chimeric transcripts.

### Example 2: Proteins Encoded by Defective Viral Genomes are Dominant-Negative Inhibitors of the Influenza Virus Polymerase

Productive infections by RNA viruses requires faithful replication of the entire genome. Many RNA viruses also produce deletion-containing viral genomes (DelVGs), genomes with large internal deletions. DelVGs can disrupt infection by WT virus, and their presence is associated with better clinical outcomes. DelVGs retain signals for replication and packaging, leading to the hypothesis that the DelVG RNA itself competes for resources. As disclosed herein, proteins encoded by DelVGs contribute to their inhibitory activity. Analysis of ribosome profiling data showed translation of chimeric viral mRNAs templated by DelVGs, especially those coding for PB1, PB2, and PA. This corresponded with DelVGs present in viral stocks, raising the possibility that ribosomes were accessing alternative reading frames by translating DelVGs. To test this, a reporter virus was prepared that reads out protein production in all three frames from a DelVG. This virus revealed multiple protein products translated from PB2 DelVGs. DelVGs cloned from the PB2 segment were included in polymerase activity assays where they reduced viral polymerase function. PB2 DelVG proteins co-immunoprecipitated PB1, creating non-functional polymerase complexes. The inhibitory effects of these proteins on viral replication were tested. The studies indicate that protein products translated from DeIVG RNAs are dominant-negative inhibitors of polymerase activity and viral replication. These findings reveal a dual inhibitory mechanism for DelVGs at both the RNA and protein level, and may support new treatments for viral infections that exploit the inhibitory nature of these protein products.

### Example 3

Thus, DVGs are not simply inhibitory RNAs, but also have the potential to code for cryptic viral proteins. DVGs were identified from all eight viral genes that maintained protein coding potential. Multiple lines of evidence indicate that these proteins are produced during viral infection, in some cases making proteins that have not been previously described for influenza virus. More importantly, some of these proteins can alter viral replication. Multiple DVG-encoded proteins that function as dominant-negative inhibitors of the viral polymerase were identified. When expressed alone or in the context of infection, these proteins block polymerase activity and decrease viral replication. Viruses were prepared that express these proteins in the place of the normal WT gene and are tested for their inhibitory potential. Mechanistic assays show that these proteins compete with and disrupt assembly of the viral polymerase. Because these proteins interfere with a conserved step in viral polymerase assembly, they are likely to be broadly inhibitory across diverse influenza isolates. Similarly, since DVGs are produced to varying degrees by all influenza isolates, the production of these proteins is also likely to be a common feature across influenza viruses. Most RNA viruses produce some version of DVGs. It is possible that these too code for unappreciated proteins with new functions.

### Example 4

DIPs are known to block replication of their WT counterparts. It has been assumed that this was achieved by the DVG RNA itself. Herein it is shown that proteins encoded by DVGs also contribute to the inhibitory activity. This reveals new inhibitory mechanisms and provides additional opportunities to specifically engineer DIPs to block WT virus replication, to serve as immunostimulatory agents, or as attenuated vaccine backbones. The inhibitory activity of DIPs offers a one-two punch where both the RNA itself and the encoded protein coordinate to interfere with replication.

Viruses can be generated to encode engineered DIPs with dual function as coding for inhibitory proteins and being parasitic RNAs. These may be potent competitors during infection suppressing replication of WT virus, offering therapeutic potential against a diverse array of influenza viruses. In addition to engineering viruses with DVGs encoding inhibitory proteins, it may be beneficial to suppress protein production or their inhibitory function. DIPs arise naturally in viral populations. They are known to contaminate influenza virus stocks used for vaccine production. The inhibitory activity for DVGs and their encoded proteins identifies an additional parameter that can be employed to increase viral yield, e.g., by minimizing DVG protein production or inhibitory activity. The immunogenicity of the vaccine may also be altered by changing the composition of DIPs in the population and their protein coding potential.

### Example 5: Proteins Encoded by Defective Viral Genomes are Dominant-Negative Inhibitors of the Influenza Virus Polymerase

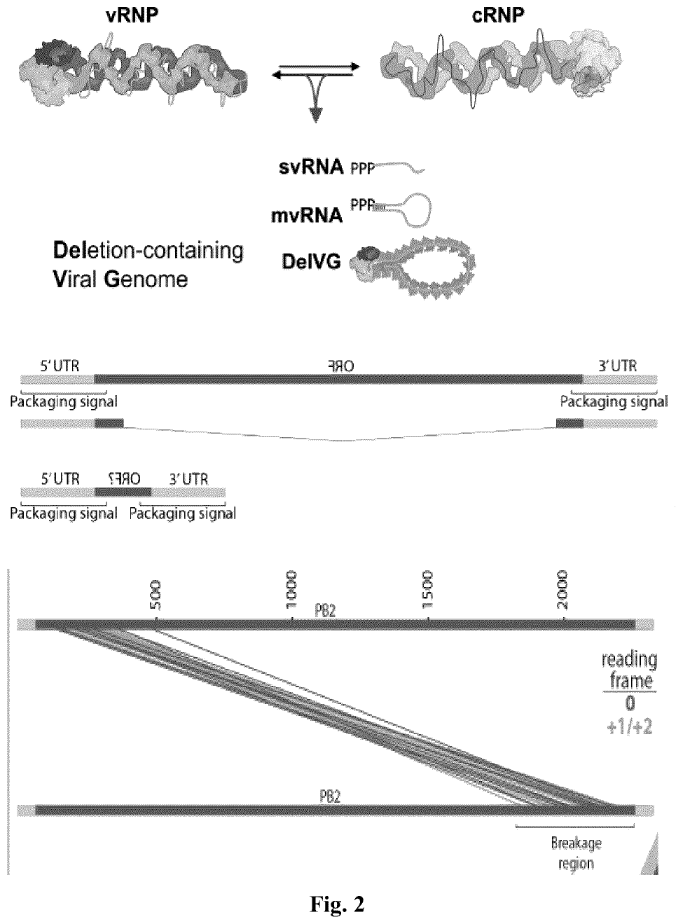

Noncanonical RNA products arise during genome replication (FIG. 2). DelVGs can maintain their UTRs and packaging signals and can be replicated and packaged into virions (FIG. 2). MappingPB2 DelVGs exposed a common breakage region and indicated many DelVGs maintain their open reading frame (FIG. 2).

It was hypothesized that deletion-containing viral genomes (DelVGs) encode proteins (DPRs) that impact influenza virus replication.

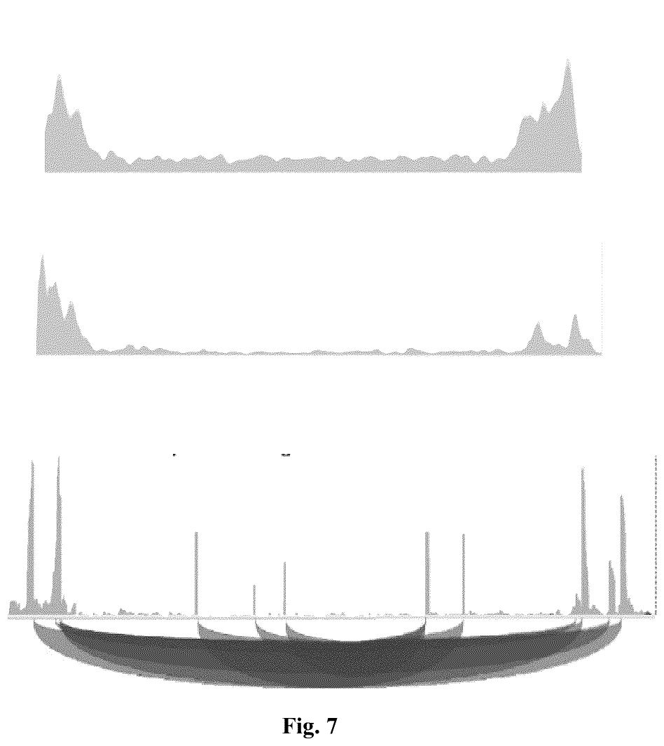

Ribo-seq revealed DelVG translation products (FIG. 7).
1) RNA-Seq shows increased read density at both ends of the PB2 genome segment, confirming the presence of DelVGs in viral stocks.
2) Ribo-seq shows a similar pattern as RNA-seq, suggesting DelVGs are actively translated by host ribosomes.
3) Mapping reveals translation of chimeric ribosome protected fragments pinpointing where translation occurs across junctions from both ends of the segment.

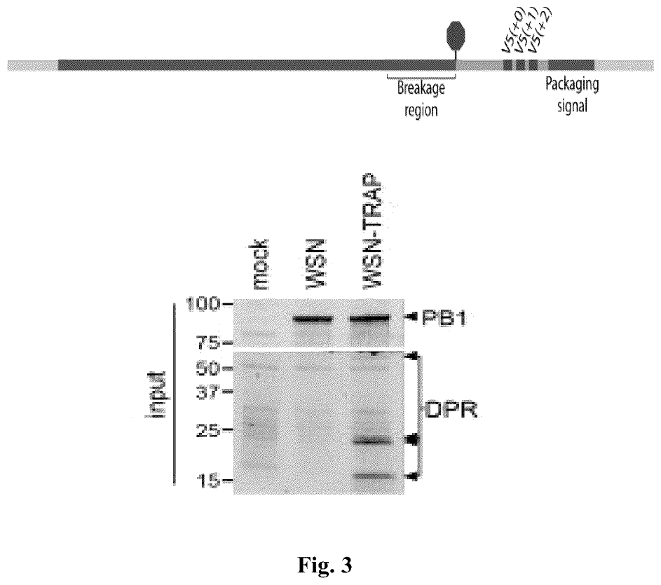

FIG. 3 shows detection of DPRs using a PB2-DPR reporter virus.

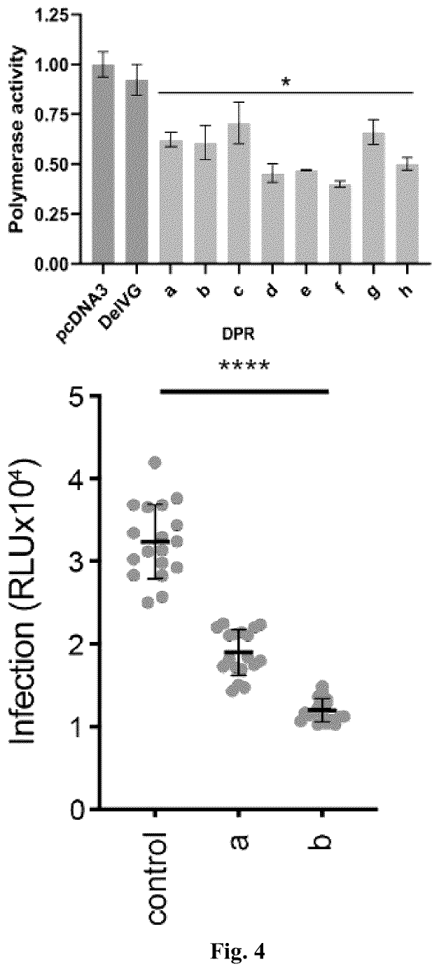

FIG. 4 demonstrates DPRs impair viral polymerase activity and replication.

PB2 DPRs were cloned from cells infected by the PB2 DPR-V5 reporter virus.

These DPRs decreased viral polymerase activity when expressed in cells. Expression of individual PB2 DPRs also inhibited influenza virus replication.

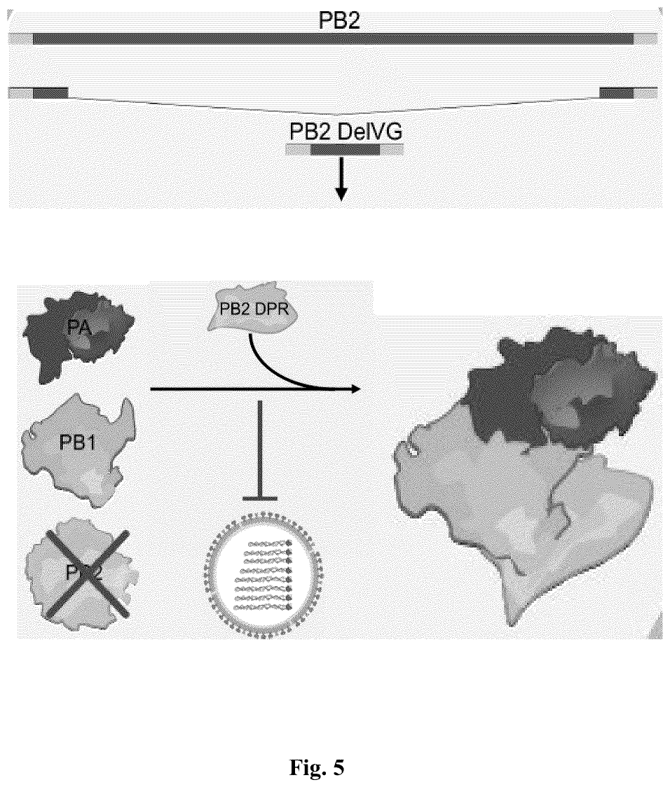

PB2 DPRs competitively inhibit polymerase assembly and impair infection (FIG. 5).

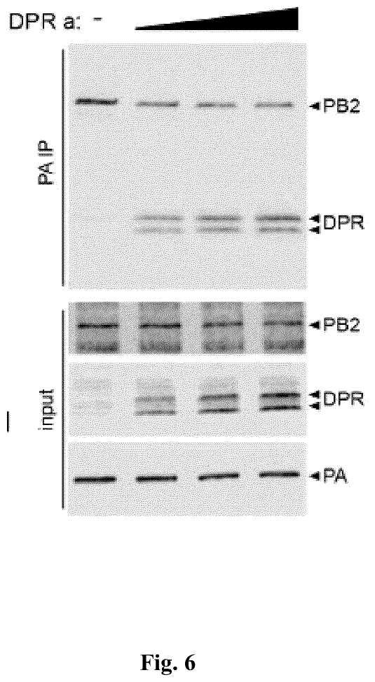

DPRs compete for trimer assembly (FIG. 6). PB2 DPRs affect assembly of the viral polymerase by competing with WT PB2 for the other subunits (PA, PB1).

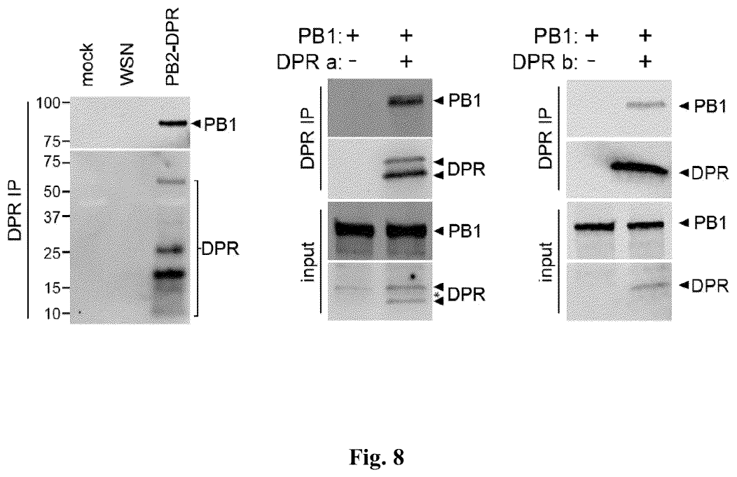

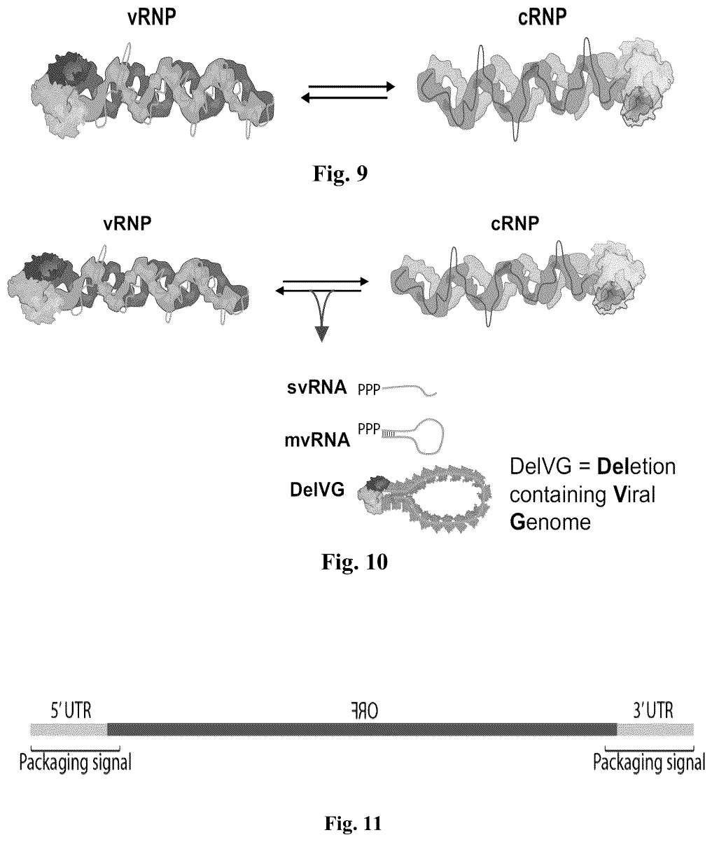

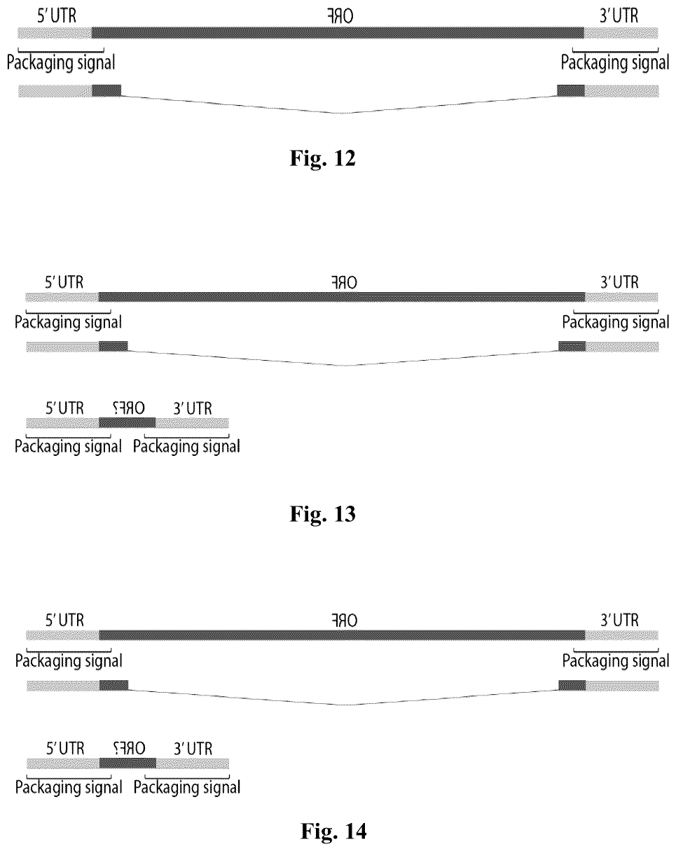

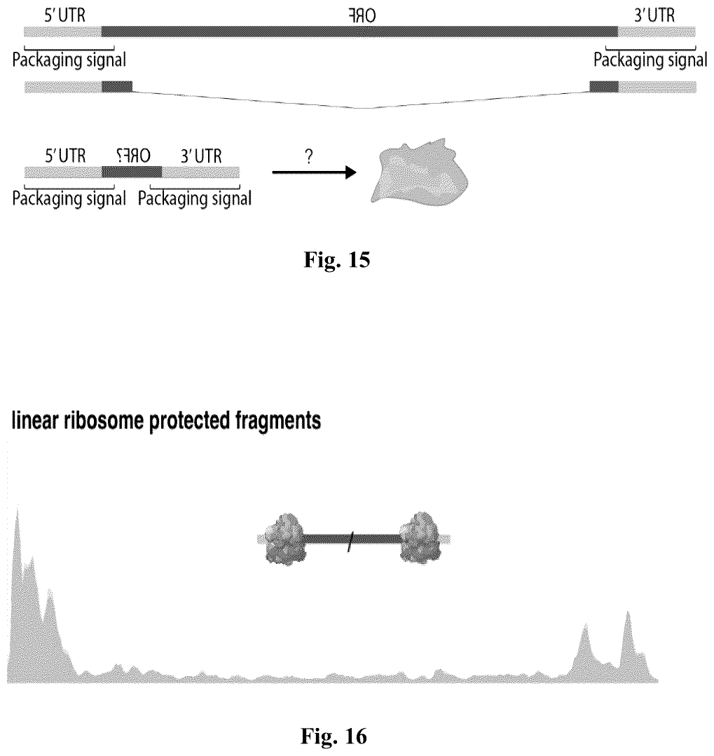

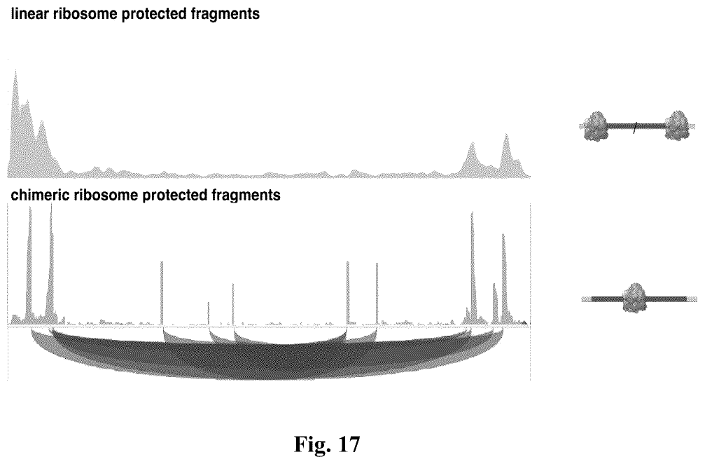

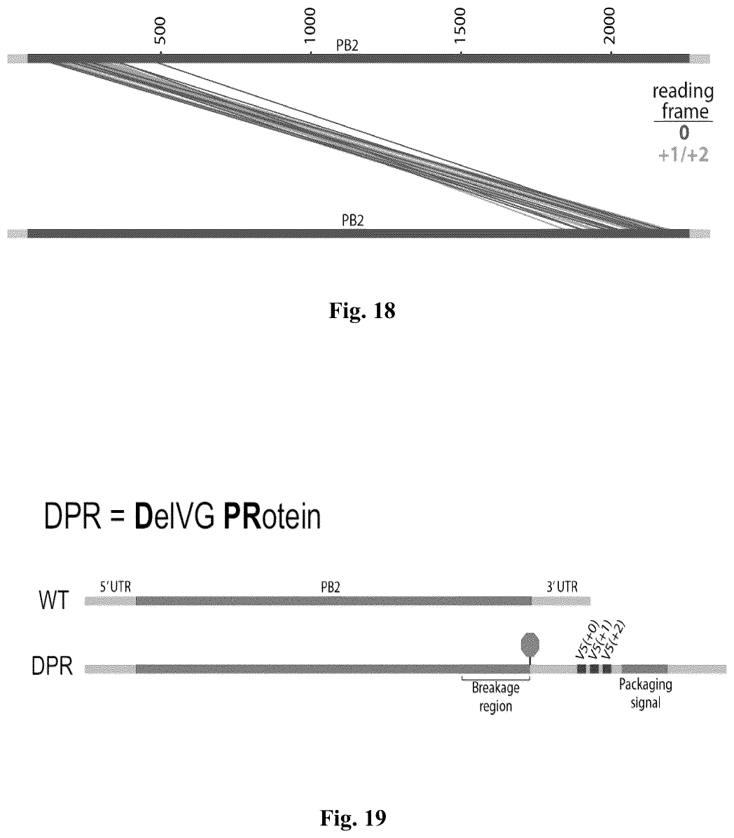

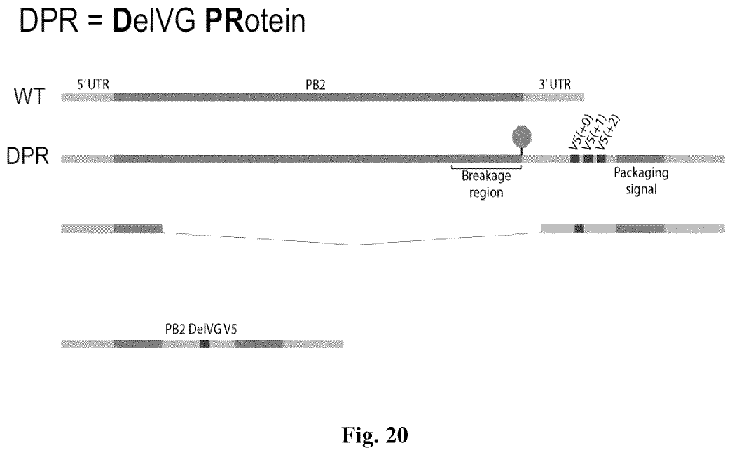

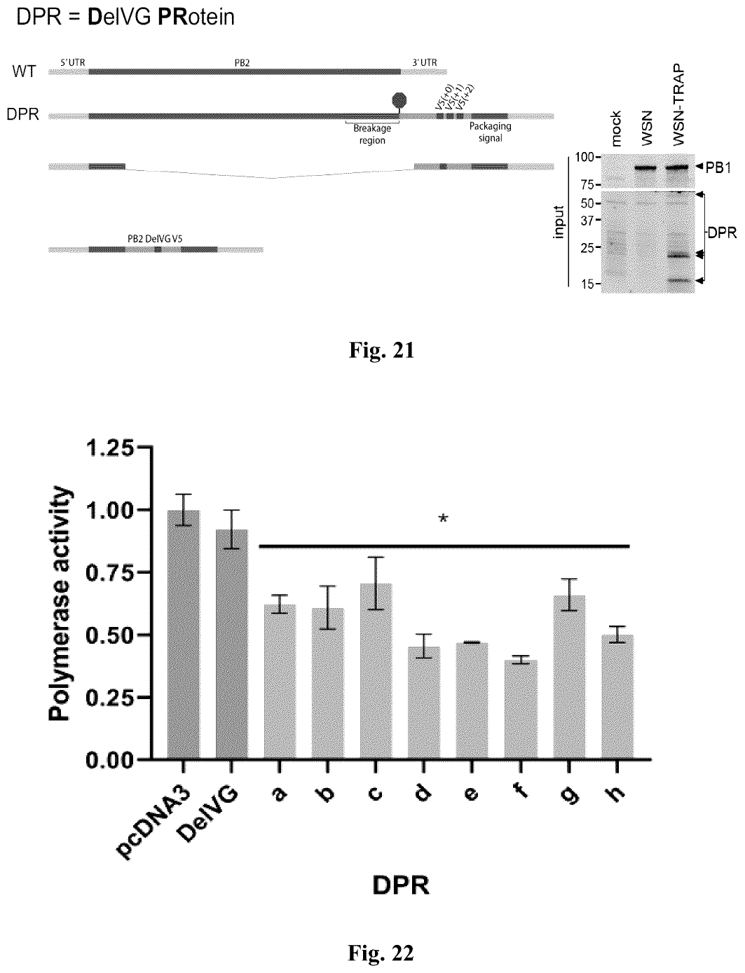

PB2 DPRs bind PB1 during infection (FIG. 8). Upon infection with the PB2-DPR reporter virus, PB1 co-immunoprecipitates with PB2 DPRs. PB2 DPRs that inhibit polymerase activity and viral replication (including “a” and “b” here) also individually interact with PB1.

TABLE 2

| | | | | Virus_ | del- | | NLS | |
|---|---|---|---|---|---|---|---|---|
| | Seg- | | | Stock_ | etion | in | (2233 | |
| DIs | ment | Start | Stop | Rep1 | (nt) | frame? | K736) | |
| PB2_109_2061 | PB2 | 109 | 2061 | 3730 | 1951 | 1 | | MERIKELRNLMSQSRTREILTKTTVDH/TQMKAQ |
| | | | | | | | | LELSPQF* (SEQ ID NO: 1) |
| PB2_205_2200 | PB2 | 205 | 2200 | 3211 | 1994 | 2 | | MERIKELRNLMSQSRTREILTKTTVDHMAIIKKY |
| | | | | | | | | TSGRQEKNPALRMKWMMAMKYPITA/ANWARR |
| | | | | | | | | RGVGNETETEL* (SEQ ID NO: 2) |
| PB2_199_2158 | PB2 | 199 | 2158 | 2213 | 1958 | 2 | | MERIKELRNLMSQSRTREILTKTTVDHMAIIKKY |
| | | | | | | | | TSGRQEKNPALRMKWMMAMKYPI/K* (SEQ ID |
| | | | | | | | | NO: 3) |
| PB2_225_2224 | PB2 | 225 | 2224 | 1597 | 1998 | 0 | Yes | MERIKELRNLMSQSRTREILTKTTVDHMAIIKKY |
| | | | | | | | | TSGRQEKNPALRMKWMMAMKYPITADKRITEM/ |
| | | | | | | | | LVMKRKRNSSILTDSQTATKRIRMAIN* (SEQ |
| | | | | | | | | ID NO: 4) |
| PB2_258_2195 | PB2 | 258 | 2195 | 1308 | 1936 | 1 | | MERIKELRNLMSQSRTREILTKTTVDHMAIIKKY |
| | | | | | | | | TSGRQEKNPALRMKWMMAMKYPITADKRITEM |
| | | | | | | | | IPERNEQGQTL/MC* (SEQ ID NO: 5) |
| PB2_187_2040 | PB2 | 187 | 2040 | 692 | 1852 | 1 | | MERIKELRNLMSQSRTREILTKTTVDHMAIIKKY |
| | | | | | | | | TSGRQEKNPALRMKWMMAMM/LAL* (SEQ ID |
| | | | | | | | | NO: 6) |
| PB2_192_2068 | PB2 | 192 | 2068 | 457 | 1875 | 0 | Yes | MERIKELRNLMSQSRTREILTKTTVDHMAIIKKY |
| | | | | | | | | TSGRQEKNPALRMKWMMAMKY/EGTAGVESA |
| | | | | | | | | VLRGFLILGKEDRRYGPALSINELSNLAKGEKAN |
| | | | | | | | | VLIGQGDVVLVMKRKRNSSILTDSQTATKRIRMA |
| | | | | | | | | IN* (SEQ ID NO: 7) |
| PB2_277_2167 | PB2 | 277 | 2167 | 168 | 1889 | 2 | | MERIKELRNLMSQSRTREILTKTTVDHMAIIKKY |
| | | | | | | | | TSGRQEKNPALRMKWMMAMKYPITADKRITEM |
| | | | | | | | | IPERNEQGQTLWSKMND/EQPCERREG* (SEQ |
| | | | | | | | | ID NO: 8) |
| PB2_217_2212 | PB2 | 217 | 2212 | 157 | 1994 | 2 | | MERIKELRNLMSQSRTREILTKTTVDHMAIIKKY |
| | | | | | | | | TSGRQEKNPALRMKWMMAMKYPITADKRI/RR |
| | | | | | | | | RGVGNETETEL* (SEQ ID NO: 9) |
| PB2_104_2035 | PB2 | 104 | 2035 | 138 | 1930 | 1 | | MERIKELRNLMSQSRTREILTKTTV/ERMLAL* |
| | | | | | | | | (SEQ ID NO: 10) |
| PB2_148_2137 | PB2 | 148 | 2137 | 115 | 1988 | 2 | | MERIKELRNLMSQSRTREILTKTTVDHMAIIKKY |
| | | | | | | | | TSGRQE/IWTSIKHK* (SEQ ID NO: 11) |
| PB2_152_2107 | PB2 | 152 | 2107 | 104 | 1954 | 1 | | MERIKELRNLMSQSRTREILTKTTVDHMAIIKKY |
| | | | | | | | | TSGRQEK/NSSFWAKKTGDMDQH* (SEQ ID |
| | | | | | | | | NO: 12) |
| PB2_163_2152 | PB2 | 163 | 2152 | 95 | 1988 | 2 | | MERIKELRNLMSQSRTREILTKTTVDHMAIIKKY |
| | | | | | | | | TSGRQEKNPAL/KHK* (SEQ ID NO: 13) |
| PB2_280_2029 | PB2 | 280 | 2029 | 74 | 1748 | 2 | | MERIKELRNLMSQSRTREILTKTTVDHMAIIKKY |
| | | | | | | | | TSGRQEKNPALRMKWMMAMKYPITADKRITEM |
| | | | | | | | | IPERNEQGQTLWSKMNDA/ARKGCWPFN* (SEQ |
| | | | | | | | | ID NO: 14) |
| PB2_128_1982 | PB2 | 128 | 1982 | 65 | 1853 | 2 | | MERIKELRNLMSQSRTREILTKTTVDHMAIIKK* |
| | | | | | | | | (SEQ ID NO: 15) |
| PB2_159_2149 | PB2 | 159 | 2149 | 64 | 1989 | 0 | Yes | MERIKELRNLMSQSRTREILTKTTVDHMAIIKKY |
| | | | | | | | | TSGRQEKNPA/LSINELSNLAKGEKANVLIGQGD |
| | | | | | | | | VVLVMKRKRNSSILTDSQTATKRIRMAIN* (SEQ |
| | | | | | | | | ID NO: 16) |
| PB2_285_1989 | PB2 | 285 | 1989 | 63 | 1703 | 2 | | MERIKELRNLMSQSRTREILTKTTVDHMAIIKKY |
| | | | | | | | | TSGRQEKNPALRMKWMMAMKYPITADKRITEM |
| | | | | | | | | IPERNEQGQTLWSKMNDAGS/SIQLQQDH* (SEQ |
| | | | | | | | | ID NO: 17) |
| PB2_238_2190 | PB2 | 238 | 2190 | 52 | 1951 | 1 | | MERIKELRNLMSQSRTREILTKTTVDHMAIIKKY |
| | | | | | | | | TSGRQEKNPALRMKWMMAMKYPITADKRITEM |
| | | | | | | | | IPER/RLMC* (SEQ ID NO: 18) |
| PB2_103_2046 | PB2 | 103 | 2046 | 50 | 1942 | 1 | | MERIKELRNLMSQSRTREILTKTTV/AL* |
| | | | | | | | | (SEQ ID NO: 19) |
| PB2_449_2172 | PB2 | 449 | 2172 | 46 | 1722 | 0 | Yes | MERIKELRNLMSQSRTREILTKTTVDHMAIIKKY |
| | | | | | | | | TSGRQEKNPALRMKWMMAMKYPITADKRITEM |
| | | | | | | | | IPERNEQGQTLWSKMNDAGSDRVMVSPLAVTW |
| | | | | | | | | WNRNGPVTSTVHYPKIYKTYFEKVERLKHGTFG |
| | | | | | | | | PVHFRNQVK/ILAKGEKANVLIGQGDVVLVMKR |
| | | | | | | | | KRNSSILTDSQTATKRIRMAIN* (SEQ ID |
| | | | | | | | | NO: 20) |
| PB2_108_2056 | PB2 | 108 | 2056 | 40 | 1947 | 0 | Yes | MERIKELRNLMSQSRTREILTKTTVDH/EDPDEG |
| | | | | | | | | TAGVESAVLRGFLILGKEDRRYGPALSINELSNL |
| | | | | | | | | AKGEKANVLIGQGDVVLVMKRKRNSSILTDSQT |
| | | | | | | | | ATKRIRMAIN* (SEQ ID NO: 21) |
| PB2_309_1883 | PB2 | 309 | 1883 | 37 | 1573 | 1 | | MERIKELRNLMSQSRTREILTKTTVDHMAIIKKY |
| | | | | | | | | TSGRQEKNPALRMKWMMAMKYPITADKRITEM |
| | | | | | | | | IPERNEQGQTLWSKMNDAGSDRVMVSPLFPSQP |
| | | | | | | | | L/HQSKVERSSPH* (SEQ ID NO: 22) |
| PB2_245_2171 | PB2 | 245 | 2171 | 36 | 1925 | 2 | | MERIKELRNLMSQSRTREILTKTTVDHMAIIKKY |
| | | | | | | | | TSGRQEKNPALRMKWMMAMKYPITADKRITEM |
| | | | | | | | | IPERNE/QPCERREG* (SEQ ID NO: 23) |
| PB2_133_2025 | PB2 | 133 | 2025 | 35 | 1891 | | | MERIKELRNLMSQSRTREILTKTTVDHMAIIKKY |
| | | | | | | | | T/* (SEQ ID NO: 24) |
| PB2_328_1940 | PB2 | 328 | 1940 | 32 | 1611 | 0 | Yes | MERIKELRNLMSQSRTREILTKTTVDHMAIIKKY |
| | | | | | | | | TSGRQEKNPALRMKWMMAMKYPITADKRITEM |
| | | | | | | | | IPERNEQGQTLWSKMNDAGSDRVMVSPLAVTW |
| | | | | | | | | WN/INVRGSGMRILVRGNSPVFNYNKTTKRLTV |
| | | | | | | | | LGKDAGPLTEDPDEGTAGVESAVLRGFLILGKED |
| | | | | | | | | RRYGPALSINELSNLAKGEKANVLIGQGDVVLV |
| | | | | | | | | MKRKRNSSILTDSQTATKRIRMAIN* (SEQ ID |
| | | | | | | | | NO: 25) |
| PB2_173_2162 | PB2 | 173 | 2162 | 29 | 1988 | 2 | | MERIKELRNLMSQSRTREILTKTTVDHMAIIKKY |
| | | | | | | | | TSGRQEKNPALRMK/* (SEQ ID NO: 26) |
| PB2_175_2164 | PB2 | 175 | 2164 | 29 | 1988 | 2 | | MERIKELRNLMSQSRTREILTKTTVDHMAIIKKY |
| | | | | | | | | TSGRQEKNPALRMKW/TEQPCERREG* (SEQ ID |
| | | | | | | | | NO: 27) |
| PB2_318_2158 | PB2 | 318 | 2158 | 28 | 1839 | 0 | Yes | MERIKELRNLMSQSRTREILTKTTVDHMAIIKKY |
| | | | | | | | | TSGRQEKNPALRMKWMMAMKYPITADKRITEM |
| | | | | | | | | IPERNEQGQTLWSEMNDAGSDRVMVSPLAVT/N |
| | | | | | | | | ELSNLAKGEKANVLIGQGDVVLVMERKRNSSIL |
| | | | | | | | | TDSQTATKRIRMAIN* (SEQ ID NO: 28) |
| PB2_222_2221 | PB2 | 222 | 2221 | 27 | 1998 | 0 | Yes | MERIKELRNLMSQSRTREILTKTTVDHMAIIKKY |
| | | | | | | | | TSGRQEKNPALRMKWMMAMKYPITADKRITE/ |
| | | | | | | | | VLVMKRKRNSSILTDSQTATKRIRMAIN* (SEQ |
| | | | | | | | | ID NO: 29) |
| PB2_90_2026 | PB2 | 90 | 2026 | 25 | 1935 | 0 | Yes | MERIKELRNLMSQSRTREILT/VLGKDAGPLTEDP |
| | | | | | | | | DEGTAGVESAVLRGFLILGKEDRRYGPALSINEL |
| | | | | | | | | SNLAKGEKANVLIGQGDVVLVMKRKRNSSILTD |
| | | | | | | | | SQTATKRIRMAIN* (SEQ ID NO: 30) |
| PB2_199_1940 | PB2 | 199 | 1940 | 24 | 1740 | 0 | Yes | MERIKELRNLMSQSRTREILTKTTVDHMAIIKKY |
| | | | | | | | | TSGRQEKNPALRMKWMMAMKYPI/INVRGSGM |
| | | | | | | | | RILVRGNSPVFNYNKTTKRLTVLGKDAGPLTEDP |
| | | | | | | | | DEGTAGVESAVLRGFLILGKEDRRYGPALSINEL |
| | | | | | | | | SNLAKGEKANVLIGQGDVVLVMKRKRNSSILTD |
| | | | | | | | | SQTATKRIRMAIN* (SEQ ID NO: 31) |
| PB2_223_2242 | PB2 | 223 | 2242 | 24 | 2018 | 2 | | MERIKELRNLMSQSRTREILTKTTVDHMAIIKKY |
| | | | | | | | | TSGRQEKNPALRMKWMMAMKYPITADERITE/T |
| | | | | | | | | EL* (SEQ ID NO: 32) |
| PB2_189_2125 | PB2 | 189 | 2125 | 21 | 1935 | 0 | Yes | MERIKELRNLMSQSRTREILTKTTVDHMAIIKKY |
| | | | | | | | | TSGRQEKNPALRMKWMMAMK/EDRRYGPALSI |
| | | | | | | | | NELSNLAKGEKANVLIGQGDVVLVMKRKRNSSI |
| | | | | | | | | LTDSQTATKRIRMAIN* (SEQ ID NO: 33) |
| PB2_178_2026 | PB2 | 178 | 2026 | 20 | 1847 | 2 | | MERIKELRNLMSQSRTREILTKTTVDHMAIIKKY |
| | | | | | | | | TSGRQEKNPALRMKWM/SSRKGCWPFN* (SEQ |
| | | | | | | | | ID NO: 34) |

### Example 6

### Materials and Methods

### Cells, Viruses, and Plasmids:

Experiments were conducted on A549 (ATCC CCL-185), HEK 293T (ATCC CRL-3216), MDCK (ATCC CCCL-34), or MDCK-SIAT-TMPRSS2 cells¹⁵. MDCK-SIAT-TMPRSS2 stably expressing PB2 were generated by retroviral gene delivery of a codon-optimized PB2 gene. Cells were maintained in Dulbecco's modified Eagle's medium (DMEM; Mediatech 10-013-CV) with 10% FBS and grown at 37° in 5% CO₂.

pCDNA3-based vectors for WSN proteins and pBD bi-directional reverse genetics plasmids were previously described¹⁶. pBD-PB2-TRAP-V5 encodes a PB2 gene segment that expresses V5-tagged proteins only when translated from a DelVG. To achieve this, we followed prior work¹⁷ by first duplicating the last 109 nt of the PB2 open reading frame and placing it downstream of the native PB2 ORF to create a contiguous packing sequence. We then codon optimized the last 106 nt of the PB2 ORF to avoid direct repeats. Many of the 3′ junctions in DelVGs were located in the last 330 nt of the PB2 ORF. We inserted another copy of this 3′ junction zone downstream of the native PB2 stop codon. This repeat encoded sequence for the V5 epitope tag in all three frames at the 3′ end. It was further modified to remove stop codons upstream of the epitope tag in all three frames. In this way, native PB2 is produced under normal circumstances and can support viral replication, while V5-tagged proteins are only produced if deletions arise that create junctions in the 3′ end of the reporter than maintain an open reading frame with one of the V5 coding sequences. All sequences were confirmed by sequencing.

Promoters used are as follows (plus-sense cRNA):

Promoter region from 5′ UTR from cRNA (plus-sense genome)

| | | |
|---|---|---|
| Our WT | 1-AGCGAAAGCAGG-12 | (SEQ ID NO. 45) |
| 3-5-8 mutant | 1-AGtGgAAaCAGG-12 | (SEQ ID NO. 46) |
| 3-8 mutant | 1-AGtGAAAaCAGG-12 | (SEQ ID NO. 47) |
| Other WT | 1-AGCaAAAGCAGG-12 | (SEQ ID NO. 48) |
| 3-5-8 mutant | 1-AGtagAAaCAGG-12 | (SEQ ID NO. 49) |
| 3-8 mutant | 1-AGtaAAAaCAGG-12 | (SEQ ID NO. 58) |

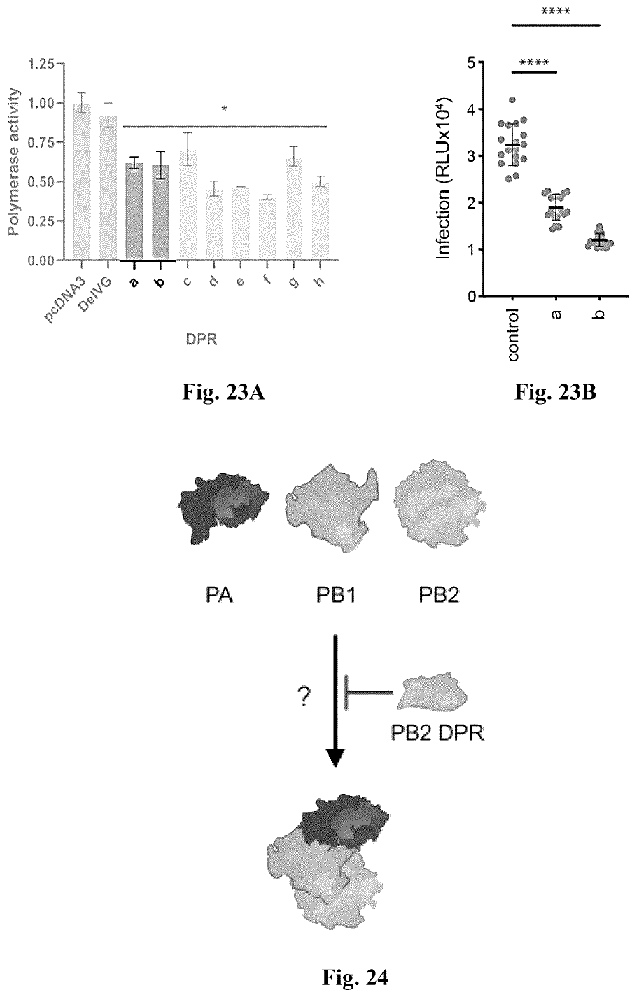

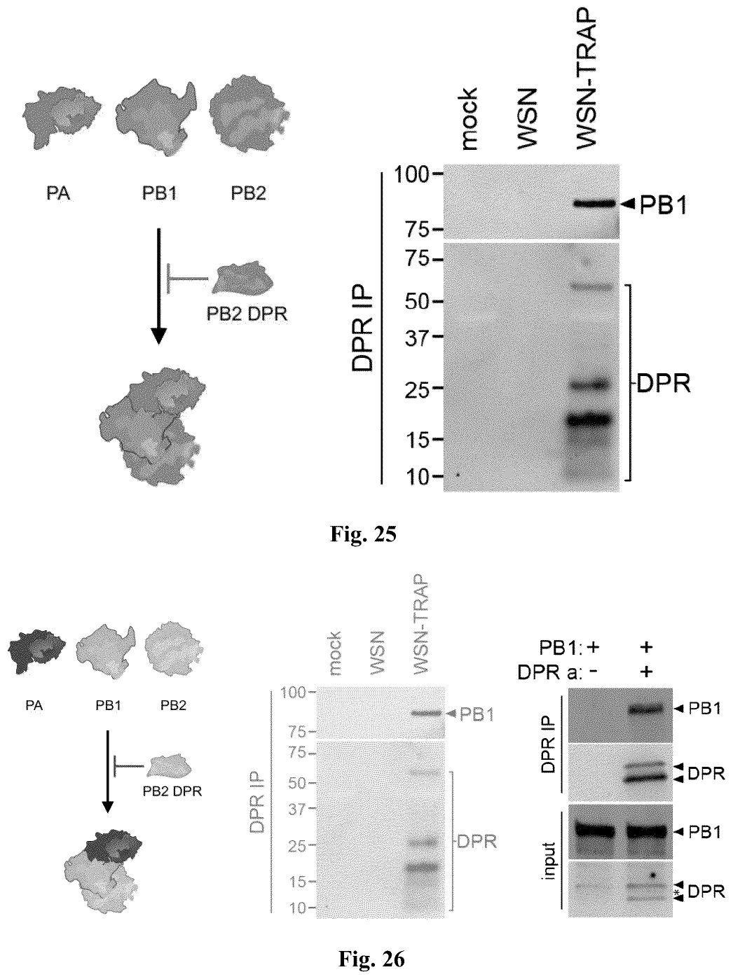

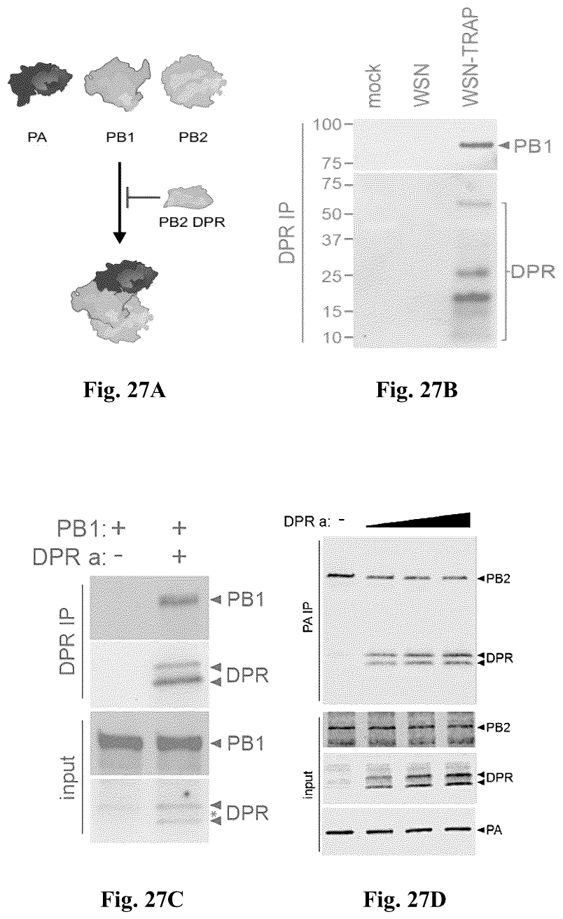

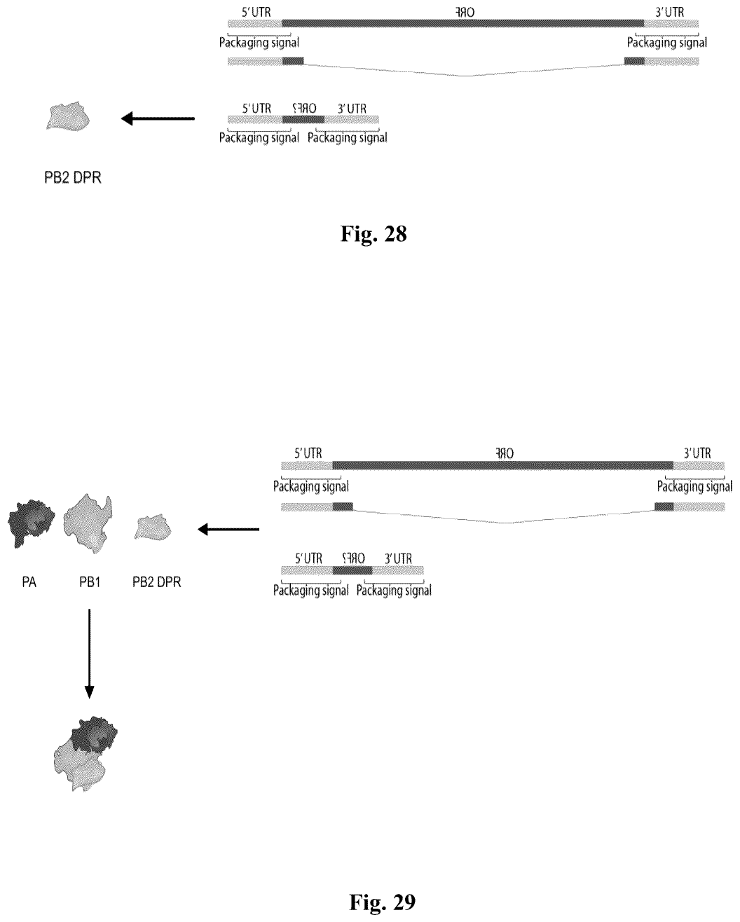

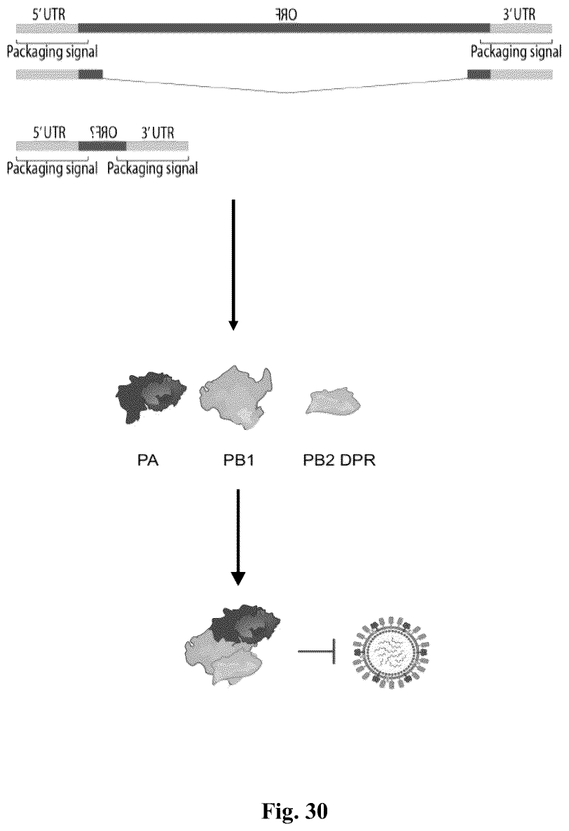

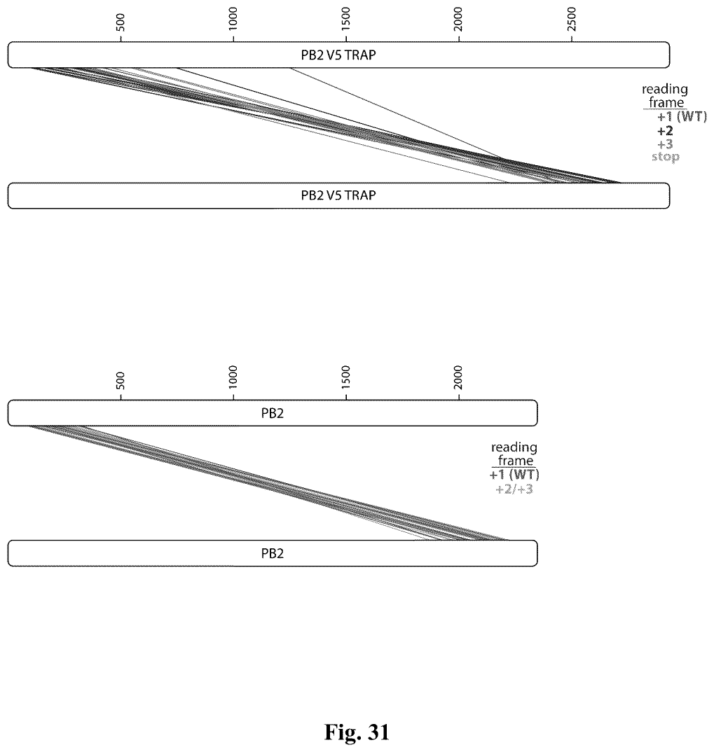

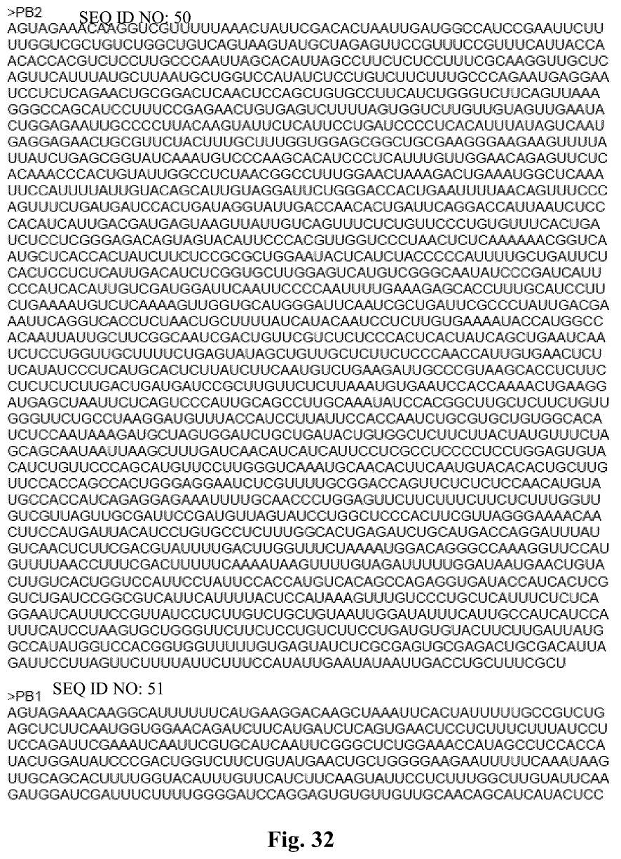

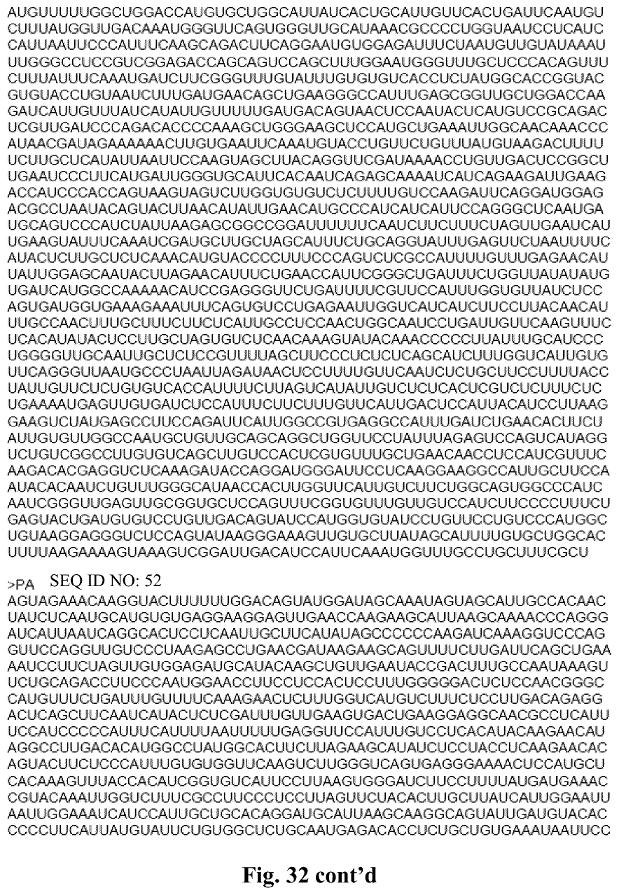

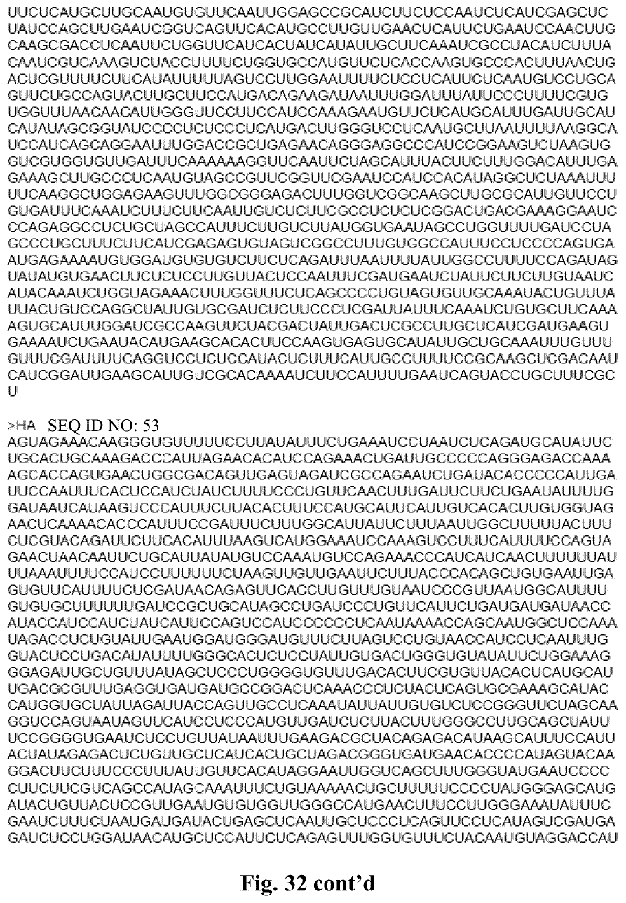

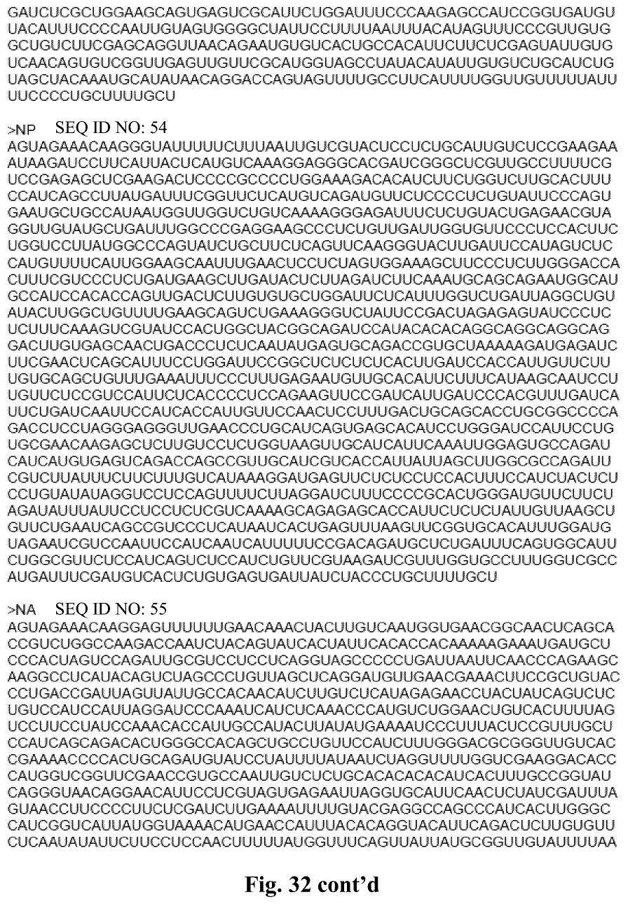

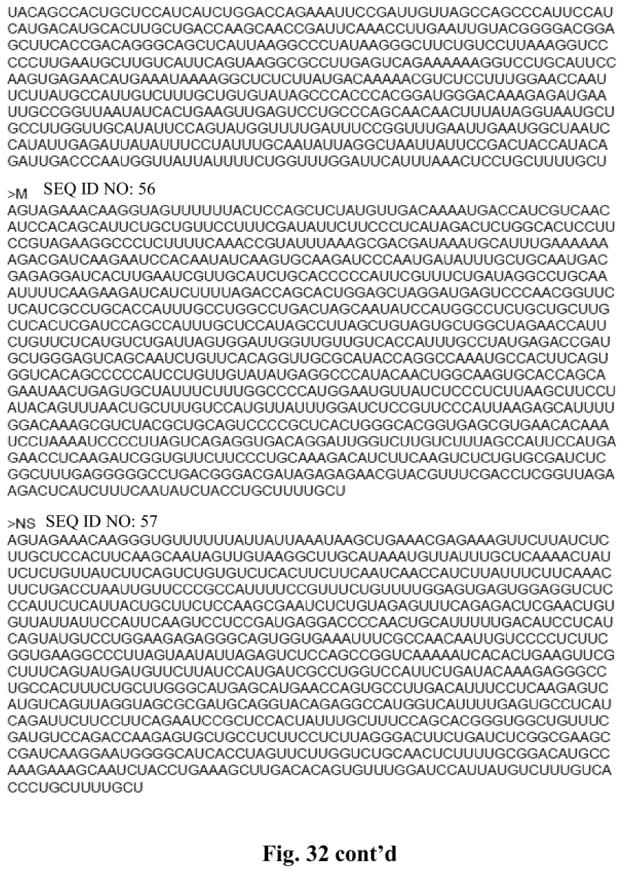

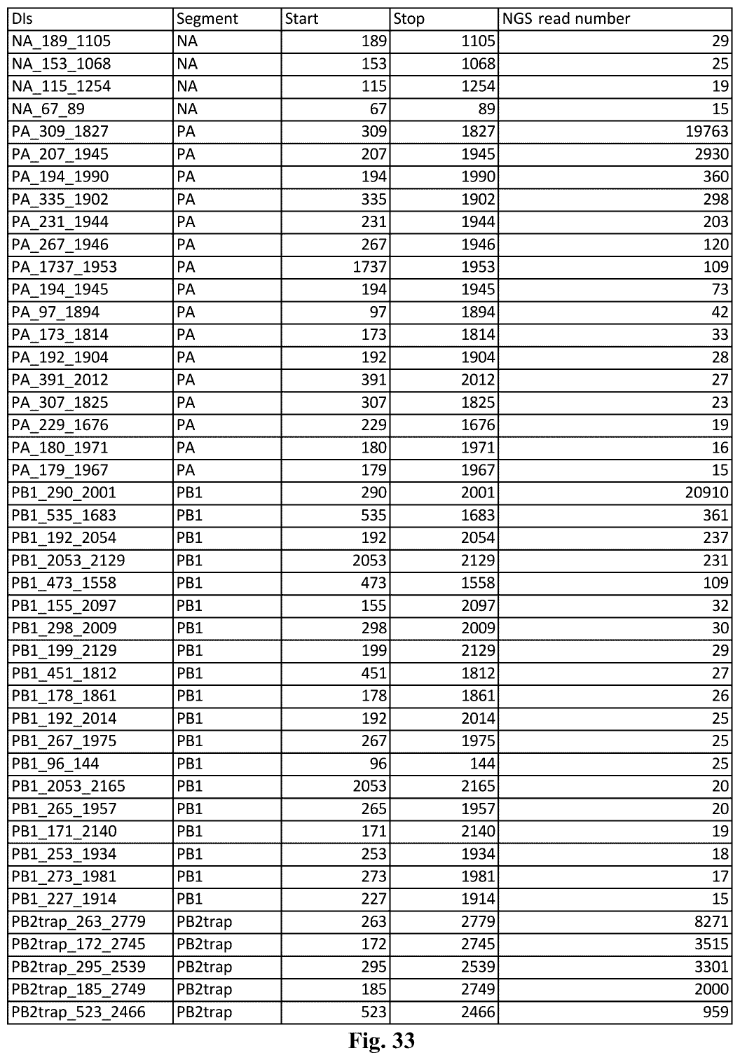

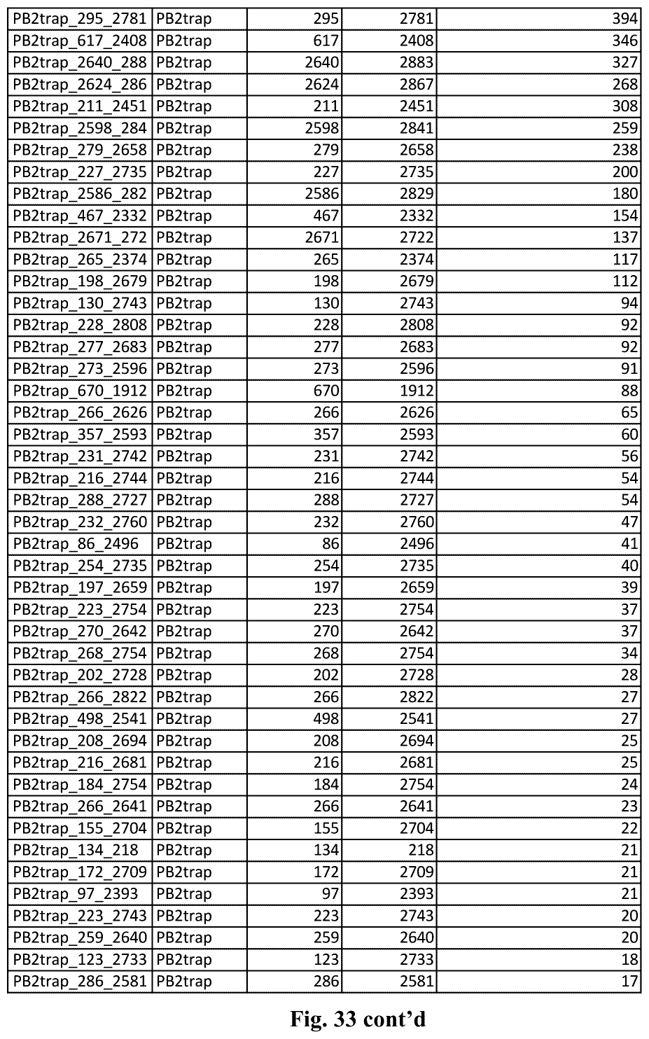

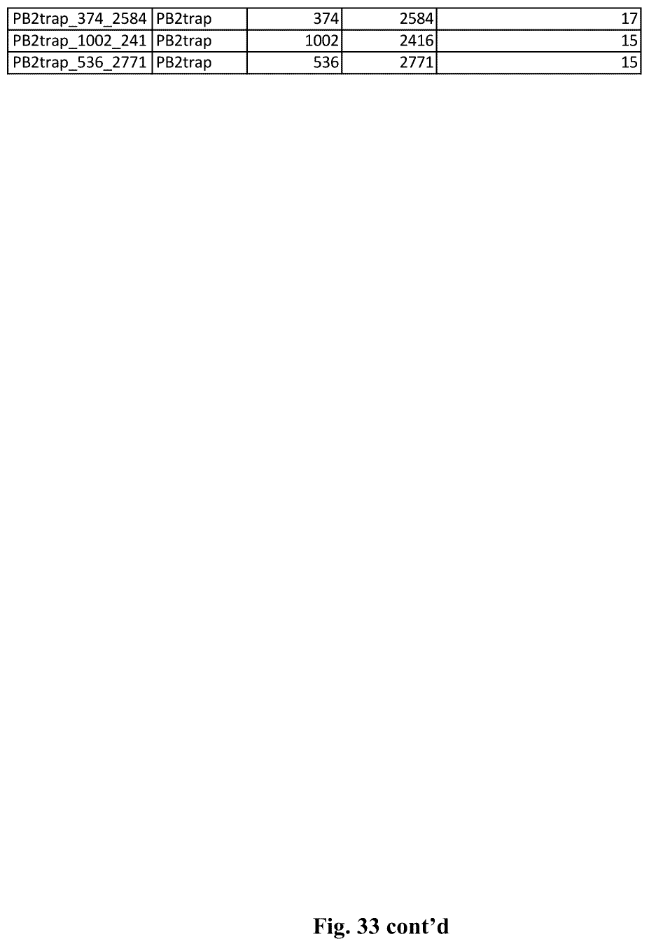

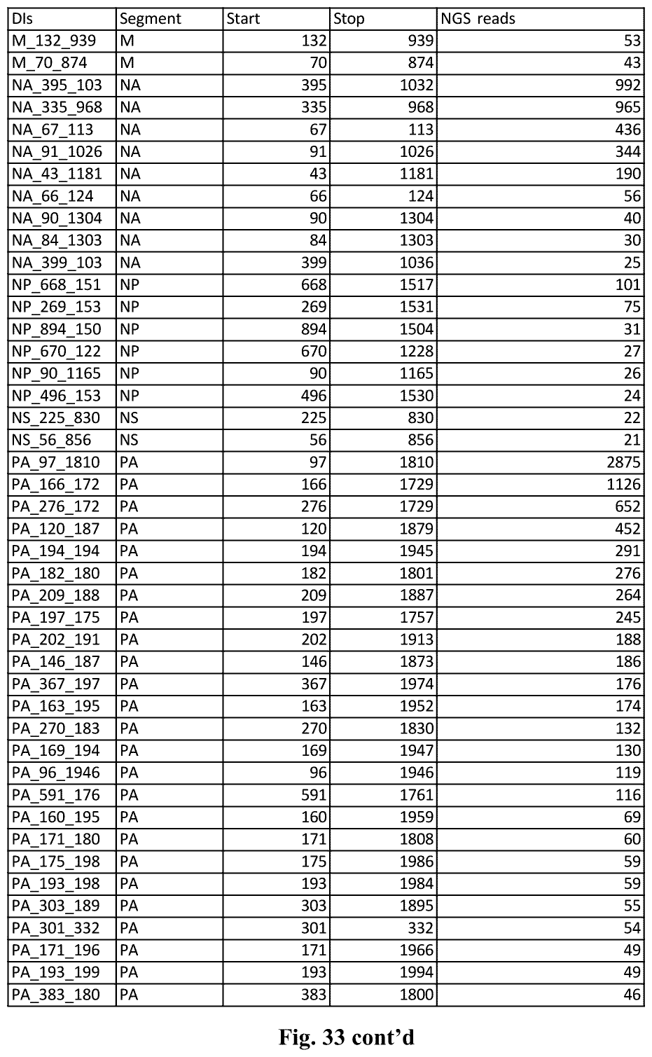

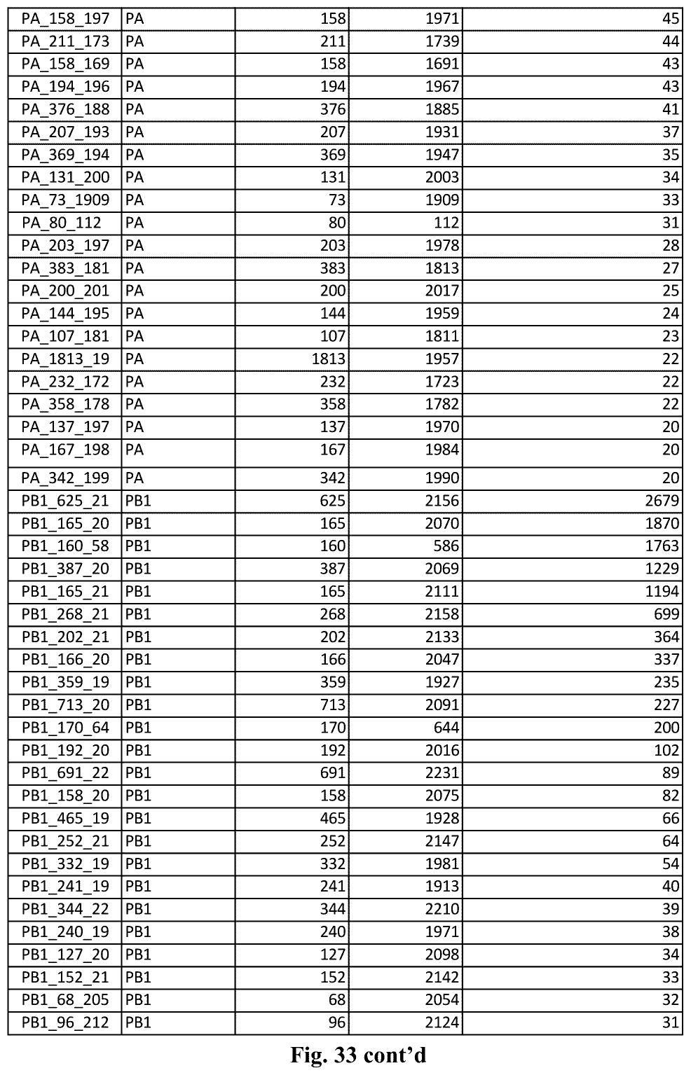

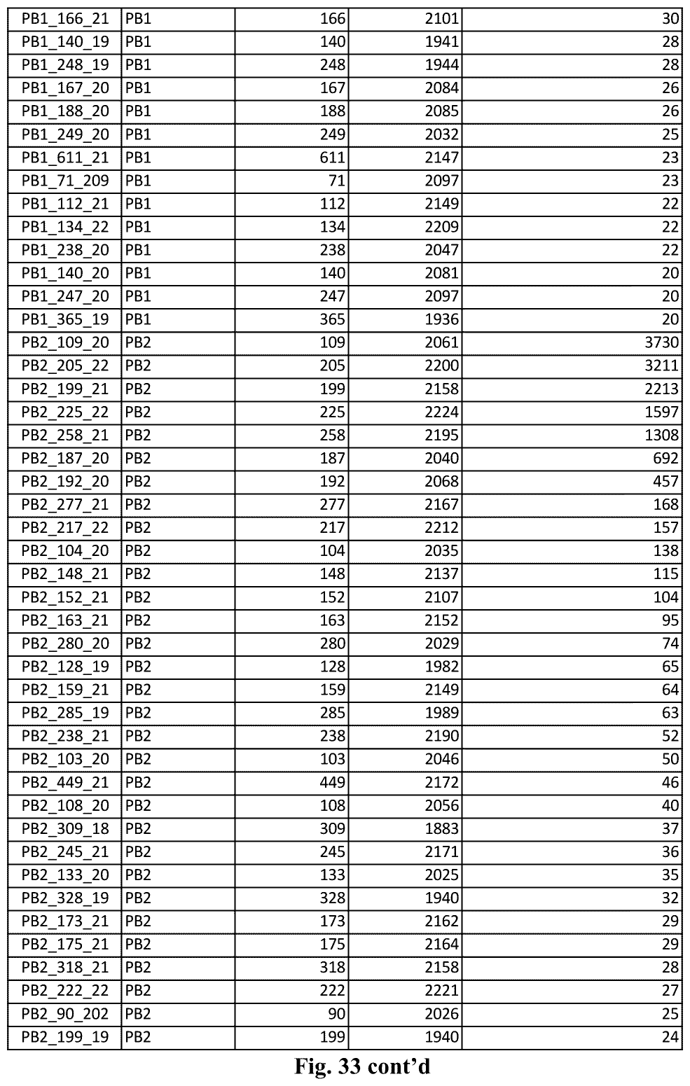

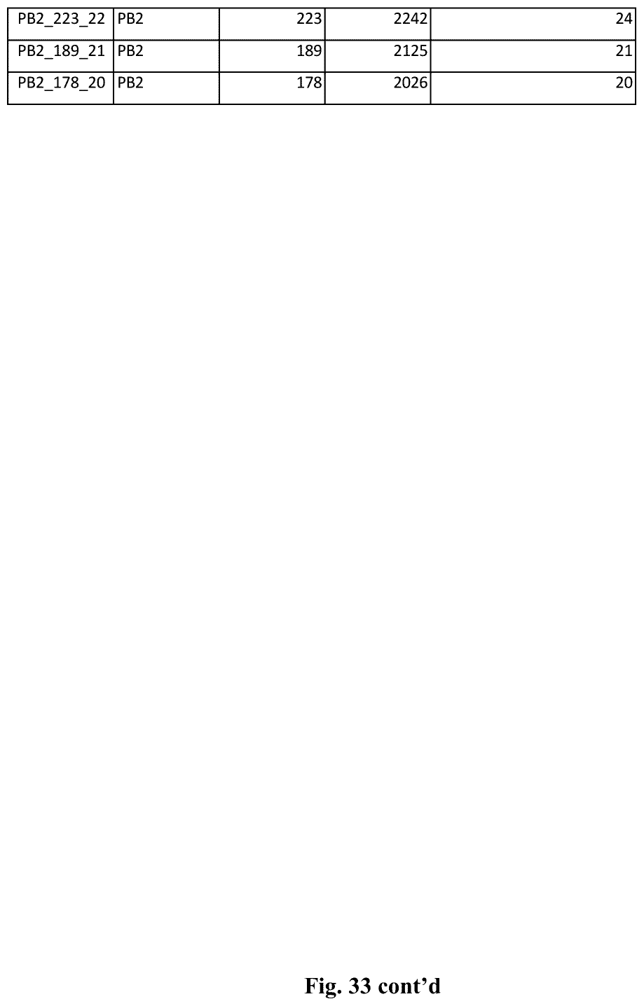

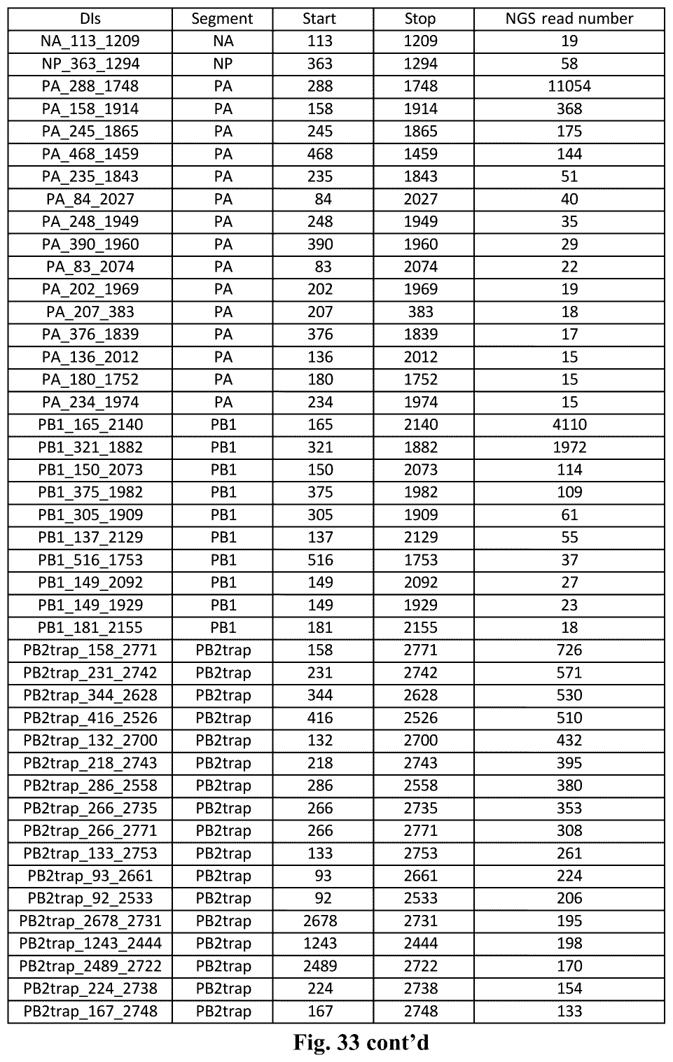

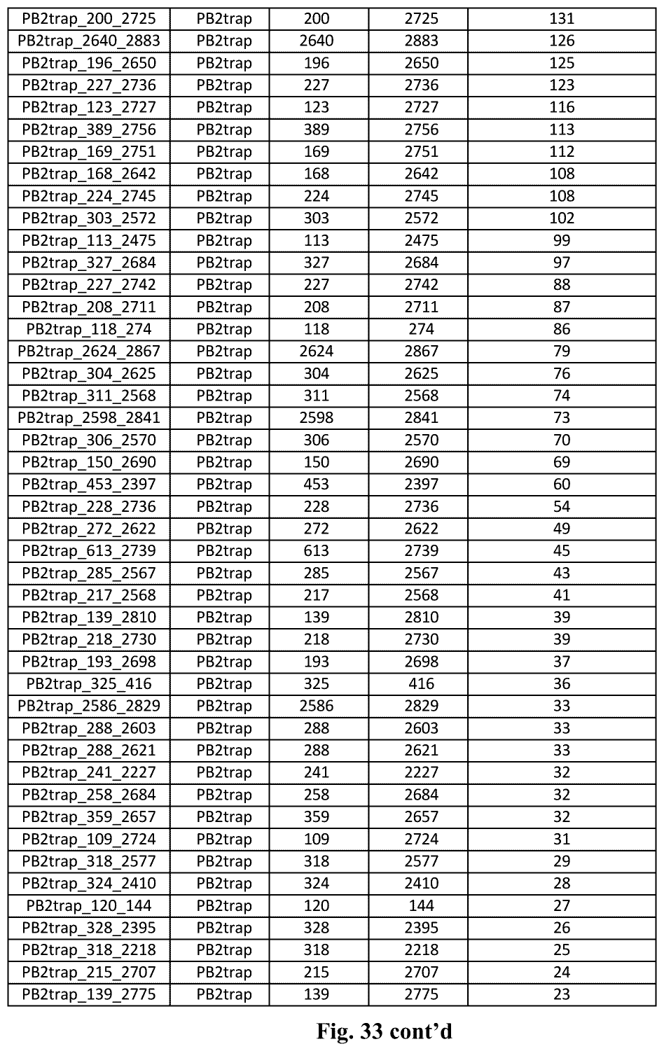

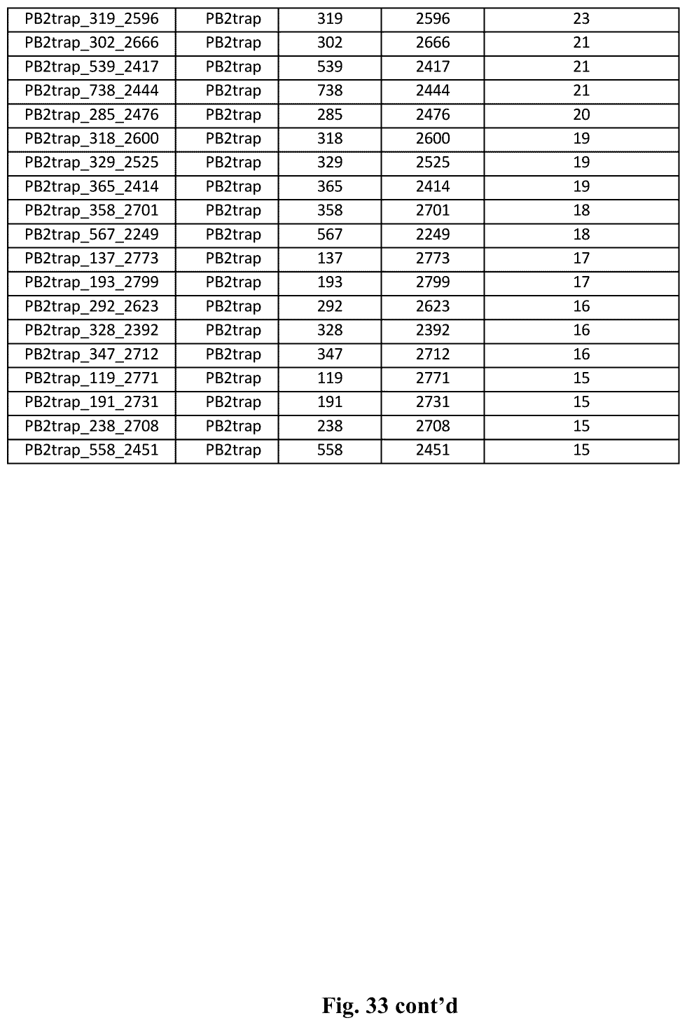

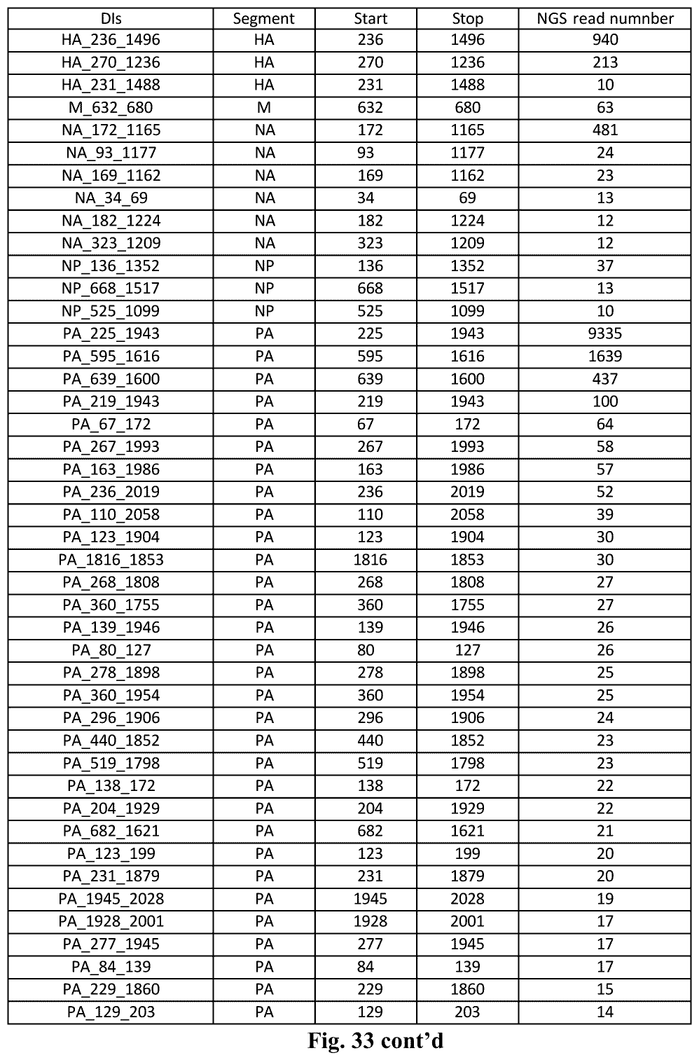

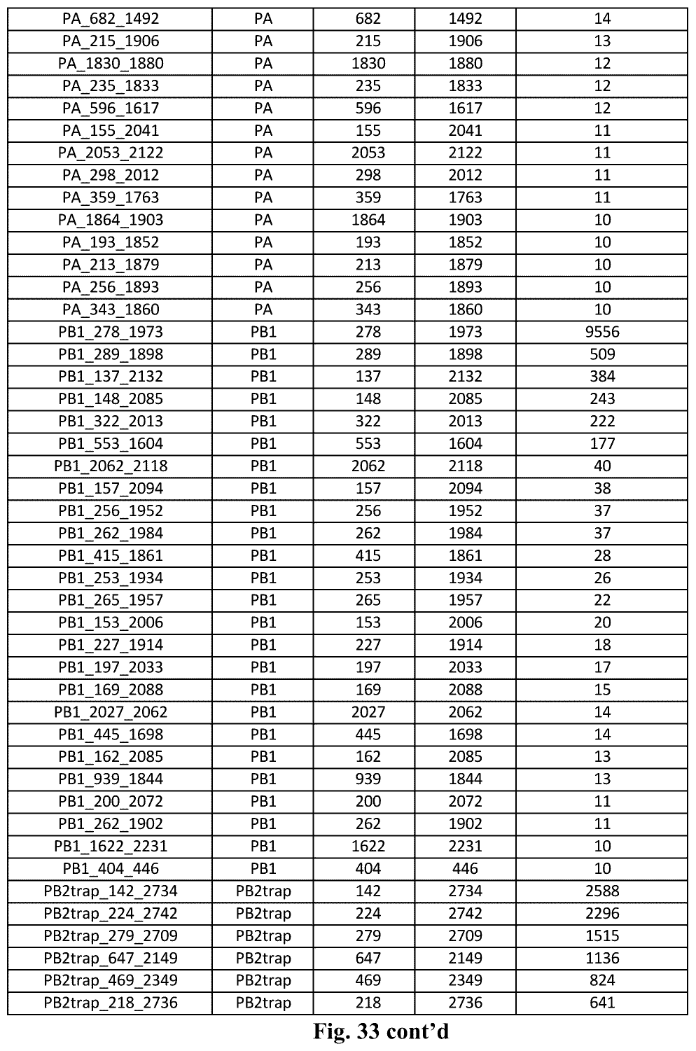

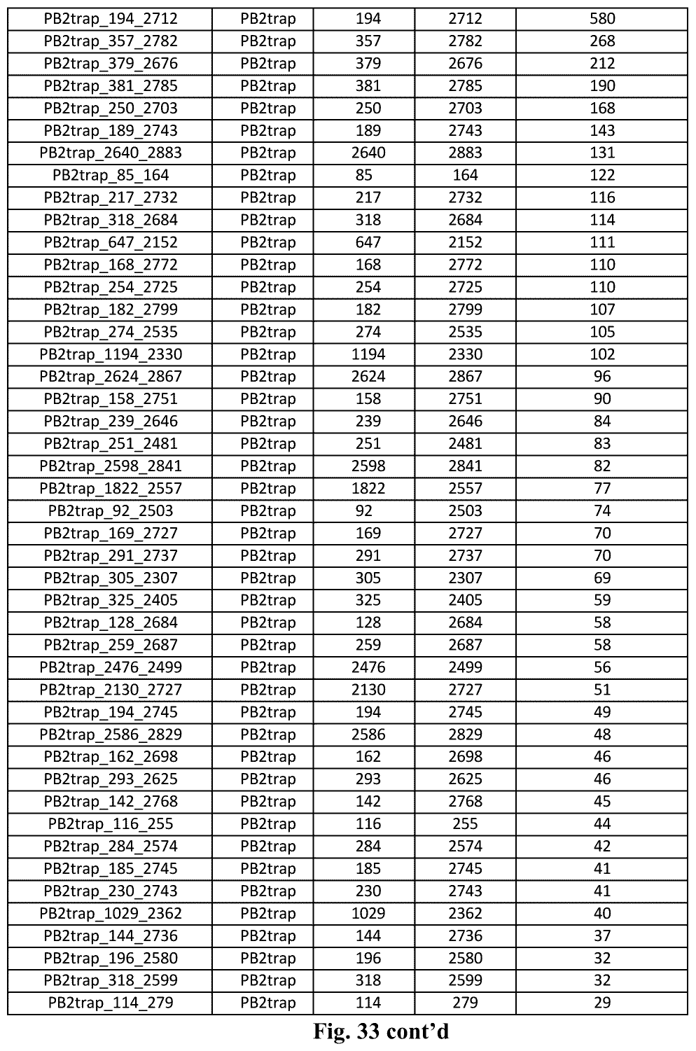

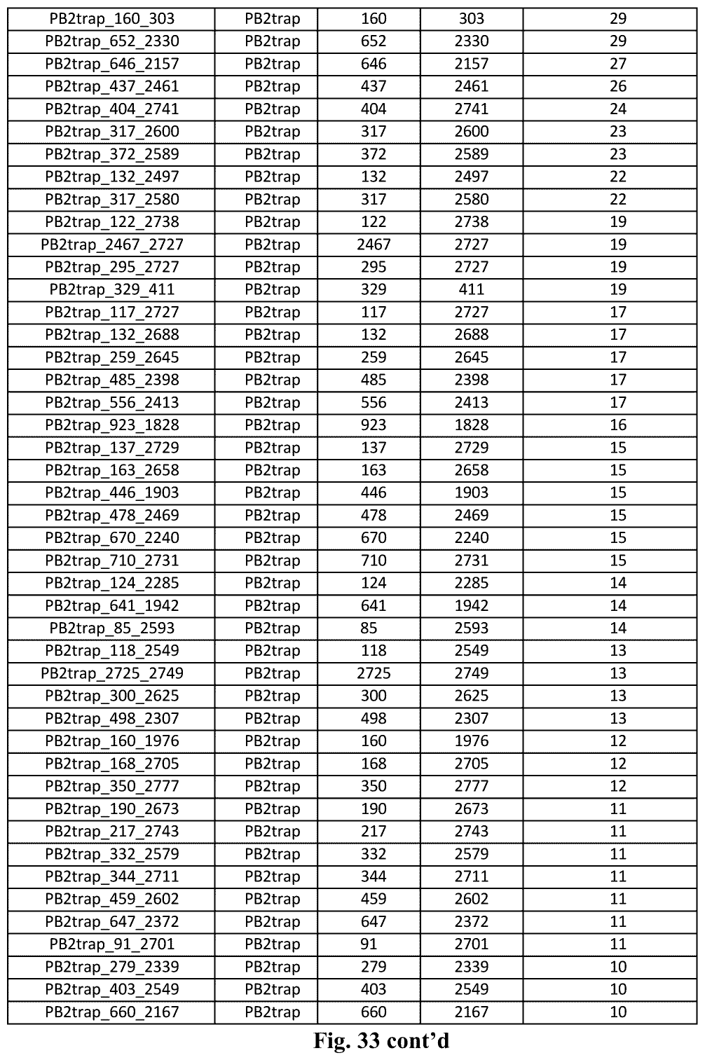

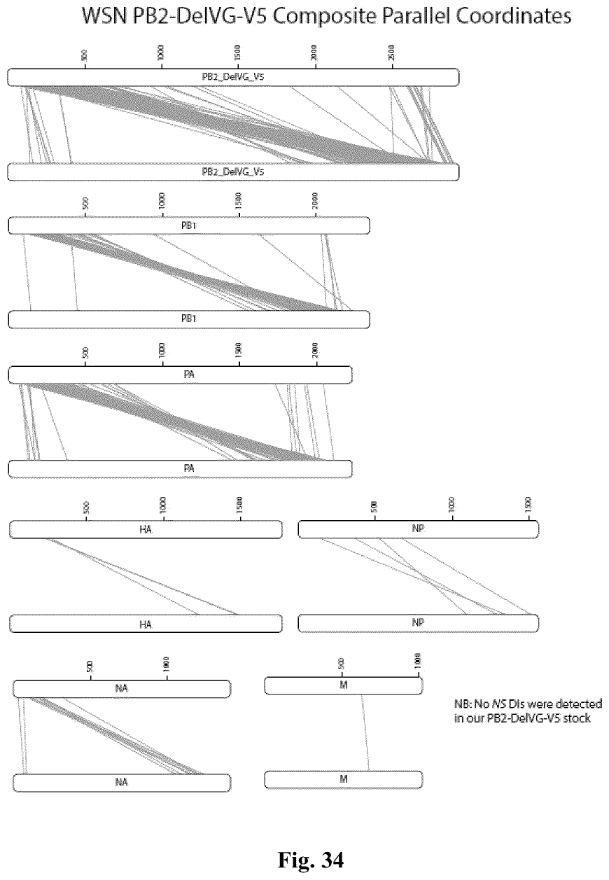

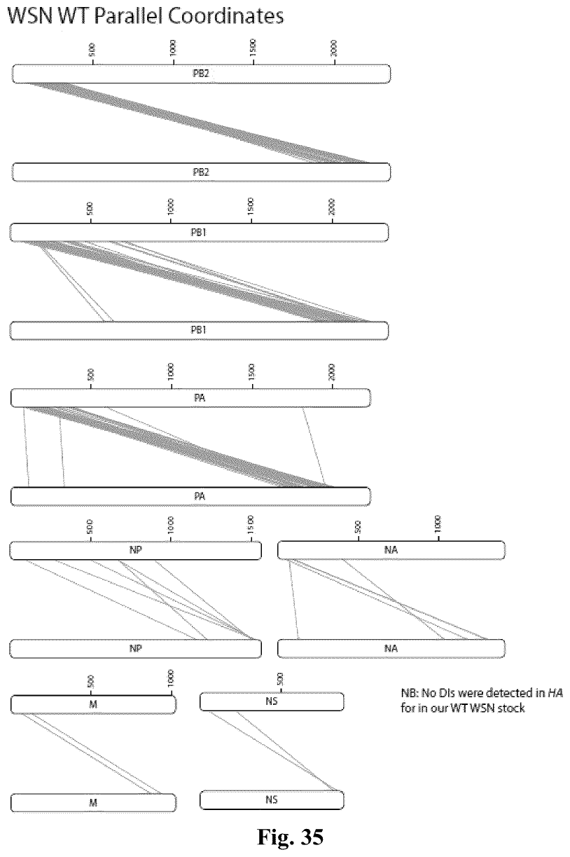

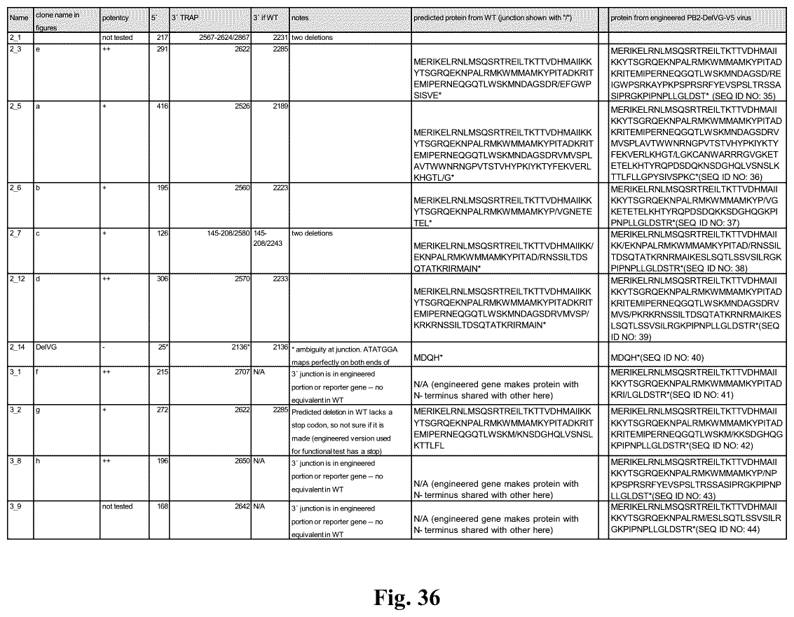

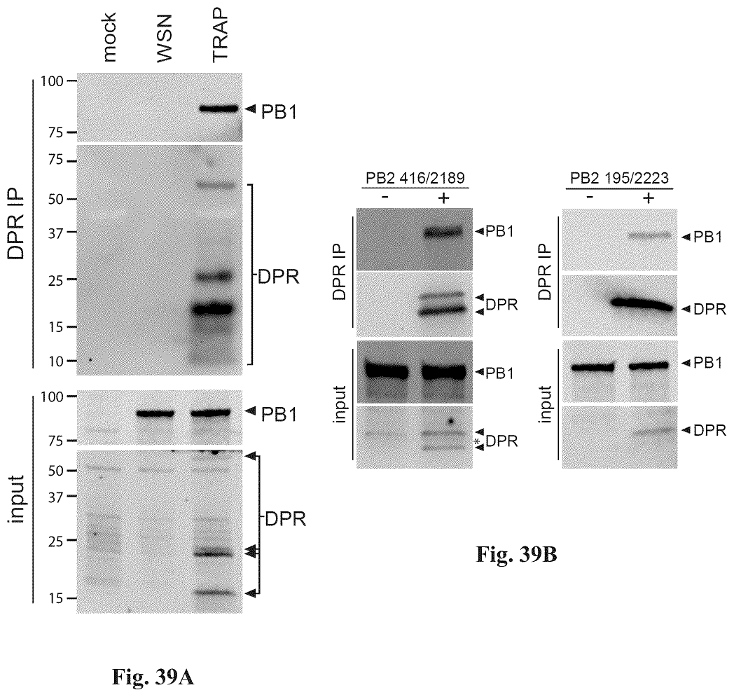

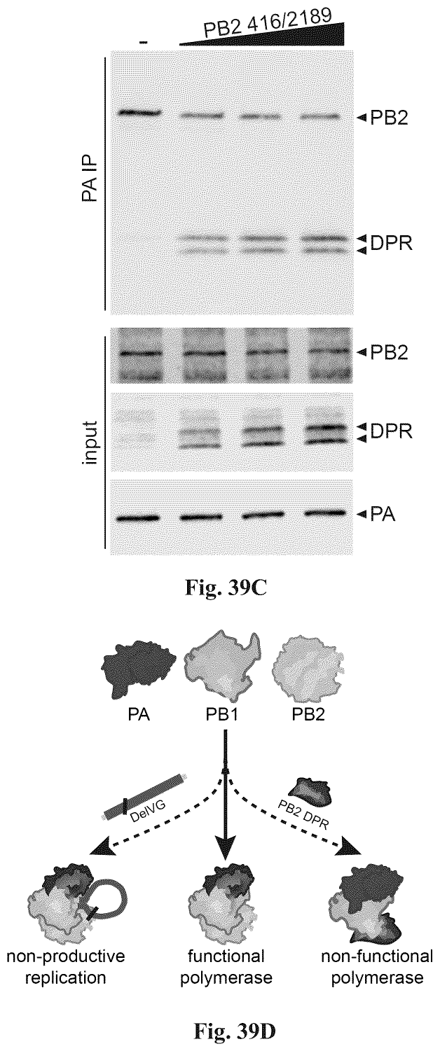

WT 416/2189
(also called 2-5, and shown as “a” in FIG. 4, FIG. 22, FIG. 23. WT is called 416/2189
in FIG. 39. FIG 36 ties all these names together in the table)
AGCGAAAGCAGGTCAATTATATTCAATatggaaagaataaaagaactaaggaatctaatgtcgcagt
ctcgcactcgcgagatactcacaaaaaccaccgtggaccatatggccataatcaagaagtacacatc
aggaagacaggagaagaacccagcacttaggatgaaatggatgatggcaatgaaatatccaattaca
gcagacaagaggataacggaaatgattcctgagagaaatgagcagggacaaactttatggagtaaaa
tgaatgacgccggatcagaccgagtgatggtatcacctctggctgtgacatggtggaataggaatgg
accagtgacaagtacagttcattatccaaaaatctacaaaacttattttgaaaaagtcgaaaggtta
aaacatggaaccttaggctaatgtgctaattgggcaaggagacgtggtgttggtaatgaaacggaaa
cggaactctagcatacttactgacagccagacagcgaccaaaagaattcggatggccatcaatTAGT
GTCGAATAGTTTAAAAACGACCTTGTTTCTACT (SEQ ID NO. 67)
3-8 Mutant of 416/2189
AGtGAAAaCAGGTCAATTATATTCAATatggaaagaataaaagaactaaggaatctaatgtcgcagt
ctcgcactcgcgagatactcacaaaaaccaccgtggaccatatggccataatcaagaagtacacatc
aggaagacaggagaagaacccagcacttaggatgaaatggatgatggcaatgaaatatccaattaca
gcagacaagaggataacggaaatgattcctgagagaaatgagcagggacaaactttatggagtaaaa
tgaatgacgccggatcagaccgagtgatggtatcacctctggctgtgacatggtggaataggaatgg
accagtgacaagtacagttcattatccaaaaatctacaaaacttattttgaaaaagtcgaaaggtta
aaacatggaaccttaggctaatgtgctaattgggcaaggagacgtggtgttggtaatgaaacggaaa
cggaactctagcatacttactgacagccagacagcgaccaaaagaattcggatggccatcaatTAGT
GTCGAATAGTTTAAAAACGACCTTGTTTCTACT (SEQ ID NO. 68)
3-5-8 mutant of 416/2189
AGtGgAAaCAGGTCAATTATATTCAATatggaaagaataaaagaactaaggaatctaatgtcgcagt
ctcgcactcgcgagatactcacaaaaaccaccgtggaccatatggccataatcaagaagtacacatc
aggaagacaggagaagaacccagcacttaggatgaaatggatgatggcaatgaaatatccaattaca
gcagacaagaggataacggaaatgattcctgagagaaatgagcagggacaaactttatggagtaaaa
tgaatgacgccggatcagaccgagtgatggtatcacctctggctgtgacatggtggaataggaatgg
accagtgacaagtacagttcattatccaaaaatctacaaaacttattttgaaaaagtcgaaaggtta
aaacatggaaccttaggctaatgtgctaattgggcaaggagacgtggtgttggtaatgaaacggaaa
cggaactctagcatacttactgacagccagacagcgaccaaaagaattcggatggccatcaatTAGT
GTCGAATAGTTTAAAAACGACCTTGTTTCTACT (SEQ ID NO. 69)

All virus and virus-derived protein expression constructs are based on A/WSN/1933 except where indicated. Viruses were rescued using the pBD bi-directional reverse genetics system¹⁸. PASTN reporter viruses expressing PA-2A-NanoLuc have been described^{9,19}. PB2 TRAP V5 virus was generated by replacing wild type PB2 with PB2 TRAP V5. Three different plaque-purified stocks of PB2 TRAP V5 were grown. Virus was amplified on MDCK-SIAT1-TMPRSS2 cells and titered on MIDCK cells by plaque assay.

Clonal DVG viruses were created by substituting the PB2 gene segment with a cloned version of a DVG. PB2 protein was provided in trans during transfection of the 293T cells. Virus was amplified and titered using MDCK-SIAT-TMPRSS2 stably expressing PB2.

WSN PB2 TRAP V5 virus and DVG viruses were confirmed by RT-PCR, sequencing, and western blot.

Transfections were completed using TransIT X2 following the manufacturers' recommendation.

### Antibodies

V5 proteins were captured using V5 trap agarose beads (Chromotek vSta). The following antibodies were used for western blot analysis: rabbit anti-PB1¹⁶, rabbit anti-PB2¹⁶, rabbit anti-V5 (Chromotek 14440-1-AP), and mouse anti-V5-HRP (Sigma V2260).

### RNA Sequencing

RNA was extracted using Trizol from viral stocks of WSN or three plaque-purified stock of PB2 TRAP V5. Sequencing was performed as described and DVGs were identified using a modified version of ViReMa^{1,20}. The parallel-coordinate maps were generated by adapting an example code (https://observablehq.com/@drimaria/parallel-coordinates-d3-v4) from the D3.js platform.

### Next Generation Sequencing of Viruses

A viral stock of WSN or three plaque-purified stocks of PB2 TRAP V5 were treated with 0.25 mg of RNaseA for 30 min at 37° C. Viral RNA was purified with the QIAamp Viral RNA Kit (Qiagen 52904). cDNA was synthesized using the Superscript III Reverse Transcriptase (Invitrogen 18080-044) and primed with MBTUni-12 (5′-ACGCGTGATCAGCRAAAGCAGG-3′ (SEQ ID NO: 70)). All 8 segments were amplified in a multiplex PCR is MBTUni-12 primer, MBTUni-13 primer (5′-ACGCGTGATCAGTAGAAACAAGG-3′ (SEQ ID NO: 71)) and Phusion Polymerase (NEB M0530L). PCR products were purified with PureLink PCR Purification Kit (Invitrogen K310002) and eluted in 30 μL of Nuclease-free water (Ambion). The RT-PCR procedure was repeated in duplicate for each virus sample. The PCR products were then subjected to next generation sequencing using the Illumina NovaSeq or MiSeq platforms.

### DVG Mapping and Quantification in Viral Stocks

We applied our DVG-detection pipeline¹ (available at https://github.com/BROOKELAB/Influenza-virus-DI-identification-pipeline). In addition to the standard pipeline, we increased confidence in sequence mapping by using the correlation between replicate sequencing runs of the same sample to calculate the Read Support cutoff (RSC) values¹. DVG were identified in these filtered dataset using a modified version of ViReMa^{1,20}. The presence of repeated sequence in our PB2 TRAP V5 reporter raised the possibility that DVG assignments may be miscalled. In addition to the standard 25 nt seed used for mapping, we re-ran the analysis using a 52 nt seed, the minimal length needed for unique mapping in the reporter. Only two reads were miscalled and they were excluded from further analysis. The parallel-coordinate maps were generated by adapting an example code (https://observablehq.com/@drimaria/parallel-coordinates-d3-v4) from the D3.js platform.

### Identification of DVGs in Ribo-Seq Data

Ribosome profiling experiments were previously reported in Tran, Ledwith, et al. 2021 (BioProject PRJNA633047)³. Data were reanalyzed with a modified version of ViReMa²⁰ paying attention to read depth at the termini of gene segments and to specifically identify and quantify chimeric ribosome protected fragments (RPFs) spanning the deletion junction of a DVG.

### Cloning DPRs

RNA was extracted from the supernatant of MDCK cells infected with the PB2 TRAP V5 virus using Trizol (Invitrogen). RNA was reverse-transcribed with Superscript III (Invitrogen) using primers specific to the PB2 segment. cDNA was amplified via PCR using primers annealing to PB2 UTRs that would capture full-length PB2 and DVGs. Products were cloned into pCDNA3, pBD or the viral RNA expression vector pHH21 and sequenced. To create versions that contained premature stop codons, DPRs cloned into pHH21 were mutated by introducing stop codons in all three frames beginning at the fourth codon of the open reading frame.

### Co-immunoprecipitation

HEK 293T cells were transfected with plasmids expressing NP, PB2-DelVG-V5, PB2, PB1 and PA. Cells were lysed in colP buffer (50 mM Tris pH 7.4, 150 mM NaCl, 0.5% NP40 and 1× protease inhibitors) and clarified by centrifugation. PB2-V5 proteins were captured with V5 trap agarose beads and washed extensively. Precipitating proteins were detected by western blot.

### Polymerase Activity Assay

Polymerase activity assays were performed by transfecting HEK 293T cells with plasmids expressing NP, PB2, PB1 and PA and pHH21-vNA luc expressing a model vRNA encoding firefly luciferase²⁵. A plasmid expression Renilla luciferase was included as a transfection control. Where indicated, PB2-DelVG-V5 proteins were co-expressed to test their inhibitory potential. Cells were lysed 24 hours after transfection in passive lysis buffer (Promega) and firefly and Renilla luciferase activity were measured. Results were normalized to the internal Renilla control. Protein expression was assessed by western blot.

### Transfection-Infection

HEK 293T cells were seeded into a 6 well plate and transfected the next day with plasmids expressing PB2-DelVG-V5 or an empty pCDNA3 control. Cells were split 1:2 24 h after transfection. 24 h after that, cells were inoculated with the PASTN reporter virus at an MOI of 0.05. Supernatants were harvested 24 h post-infection. Viral titers were measured by infecting MDCK cells and measuring Nanoluciferase activity 8 h later with the Nano-Glo Luciferase Assay System (Promega)¹⁰.

### Statistics

Experiments were performed in at least biological triplicate, with each including at least three technical replicates. Results are presented as grand mean of three biological replicates±standard error, or mean of a representative biological replicate±standard deviation, as indicated. Statistical significance of pairwise comparisons were made with a two-tailed Student's t-test or one-sample t-test. Multiple comparisons were assessed by ANOVA with post hoc Dunnett's multiple comparisons test. The correlation between input RNA and ribosome-protected fragments was determined using a Spearman's correlation coefficient. c² tests were used to test for deviation from an expected distribution.

### Data Availability

Ribo-seq data are available in BioProject PRJNA633047. Sequencing data from viral stocks are accessible as BioProject PRINA957839.

### Results:

Translation of DelVG mRNAs

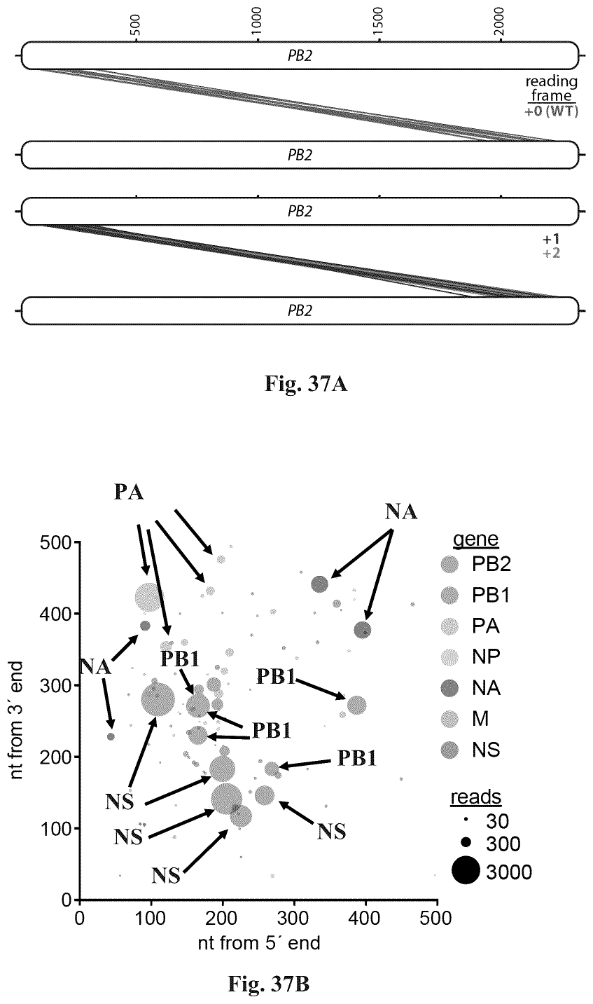

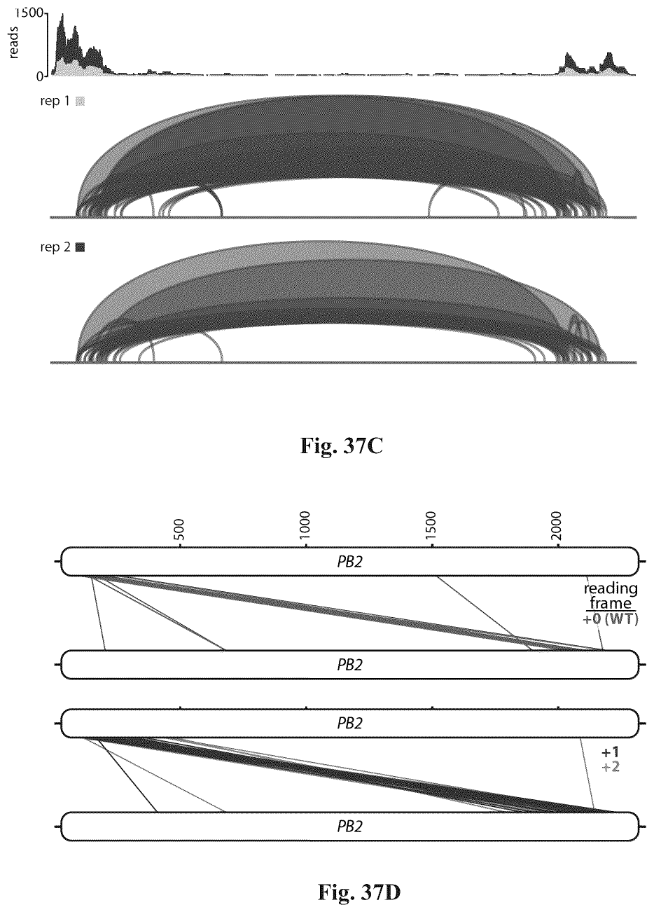

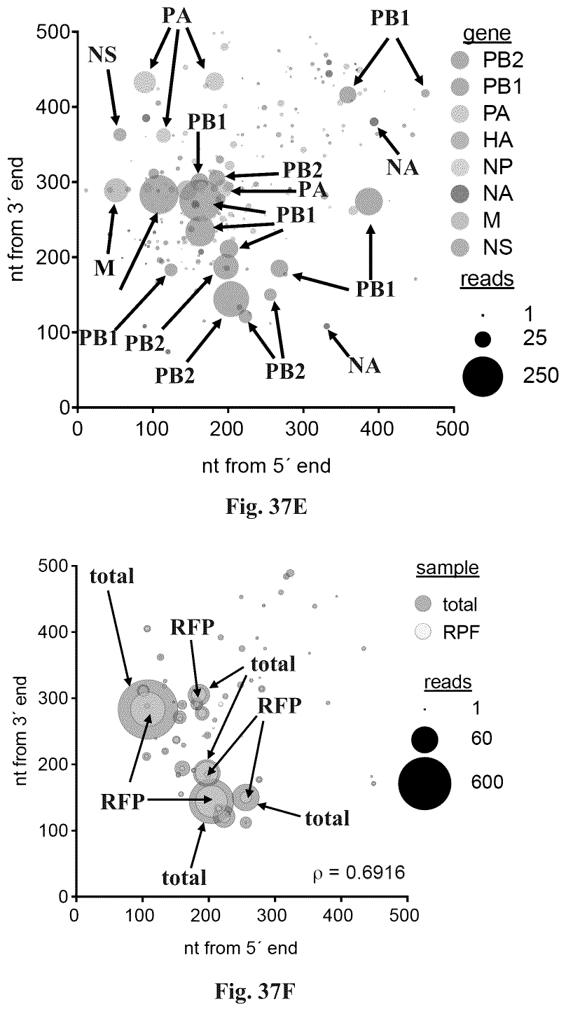

DelVGs are aberrant replication products generated by the viral polymerase during infections in vivo and in culture. DelVGs are replicated and transcribed, yet their potential to produce protein products is unclear. RNA-seq of virion-associated RNA was performed to identify and characterize the DelVGs present in viral stocks. As DelVGs contain large internal deletions, an analysis pipeline allows discontinuous mapping of reads for precise identification of deletion junctions¹. In addition to full-length vRNA, DelVGs originating from all genomic segments except HA were identified (FIG. 37A). Of the 137 DelVGs detected, 86% were derived from the polymerase gene segments, consistent with prior work showing that these larger segments are more likely to be the source of DelVGs. DelVG breakpoints were heavily concentrated near the termini, with ˜90% starting within the first 400 nt of a fragment and rejoining with the last 450 nt (FIGS. 37A and 37B). While we represent DelVGs in cRNA orientation, it is not clear whether deletions were formed during cRNA or vRNA synthesis. Further, the DelVGs that were detected are not necessarily reflective of all of those that were made, but rather those that were maintained².

Analysis of DelVG sequence revealed that 30% of the deletions maintained the original reading frame after the deletion (FIG. 37A; Tables 3A-C below), raising the possibility that mRNAs transcribed from DelVGs have the potential to produce protein products that are fusions between the N- and C-termini of the parental protein. The remaining DelVGs shifted into the +1 or +2 reading frame downstream of the junction. Transcripts from these would code for proteins with a native N-terminus followed by entirely new protein sequence downstream of the junction until encountering a stop codon that is normally out of frame. For the case of the PB2 DelVGs in our stock, this could result in the addition of up to 17 amino acids from an alternative reading frame downstream of the native N-terminus (Table 3A-C below).

TABLE 3A

| gene | total DelVGs |
|---|---|
| PB2 | 34 |
| PB1 | 38 |
| PA | 46 |
| HA | 0 |
| NP | 6 |
| NA | 9 |
| M | 2 |
| NS | 2 |
| total | 137 |

TABLE 3B

Deletion position

| | nt from 5′ | nt from 3′ |
|---|---|---|
| min | 43 | 35 |
| max | 1813 | 2122 |

TABLE 3C
reading frame after deletion

| frame | frequency |
|---|---|
| 0 | 41 |
| 1 | 42 |
| 2 | 54 |

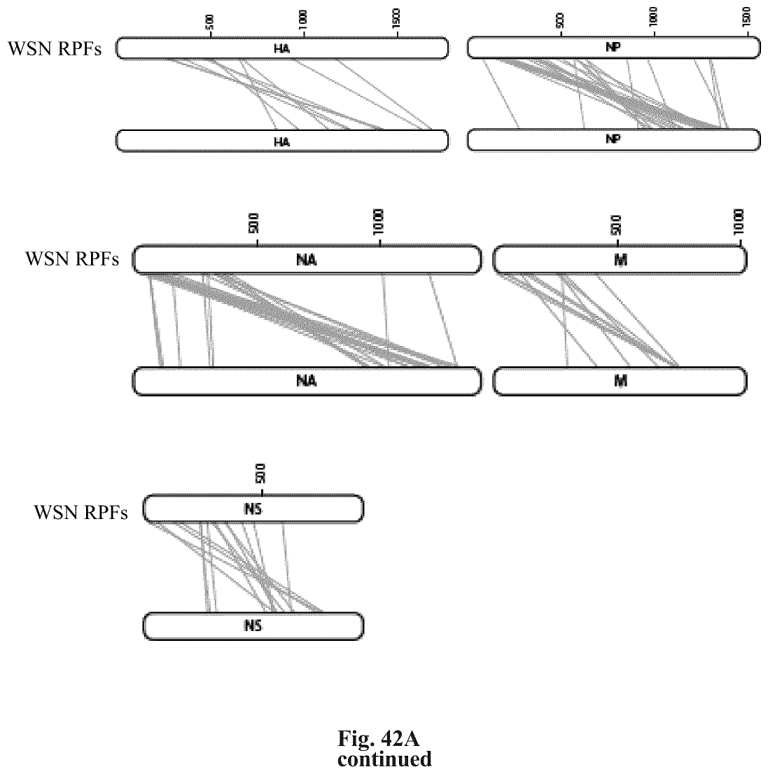

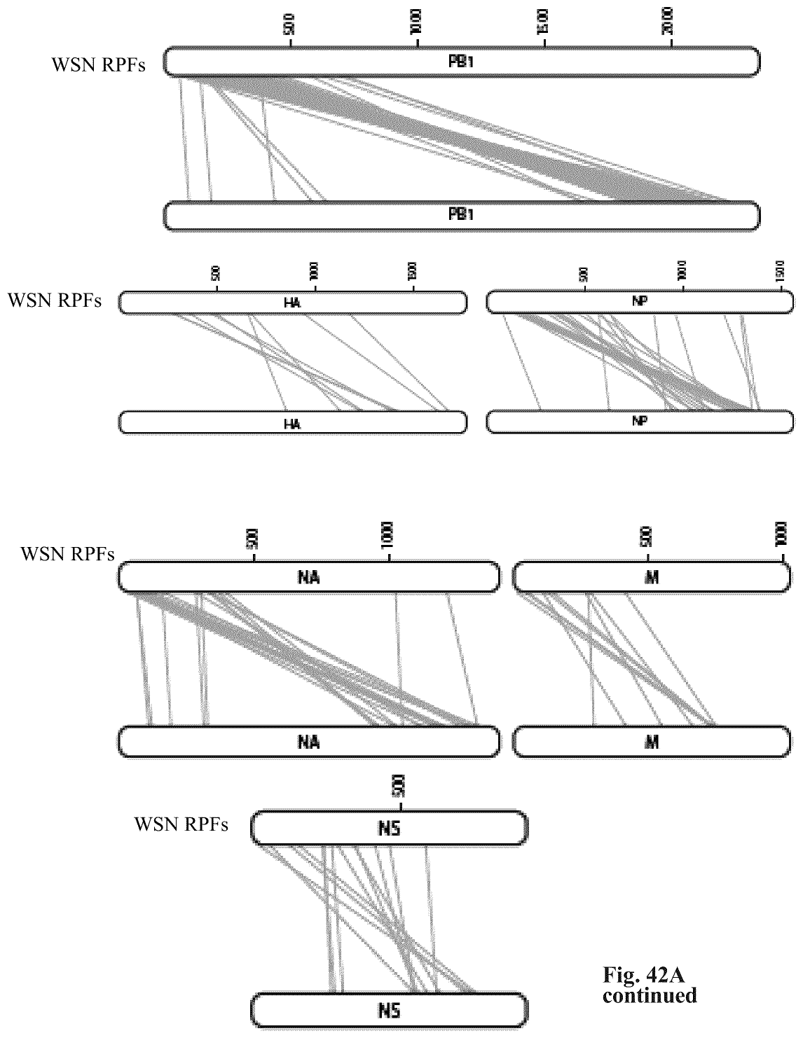

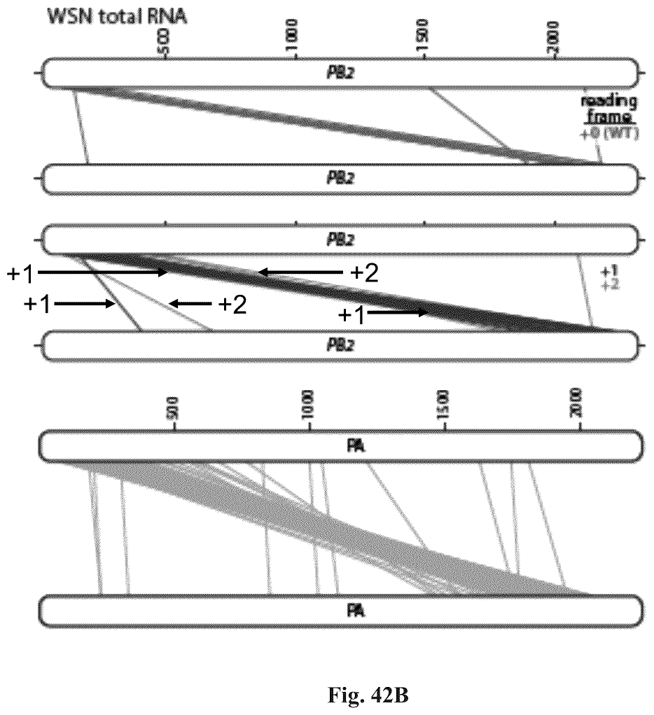

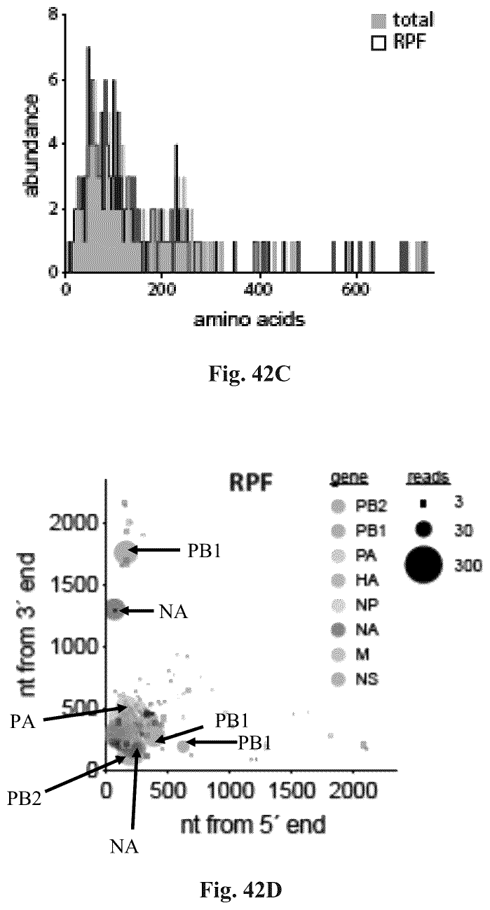

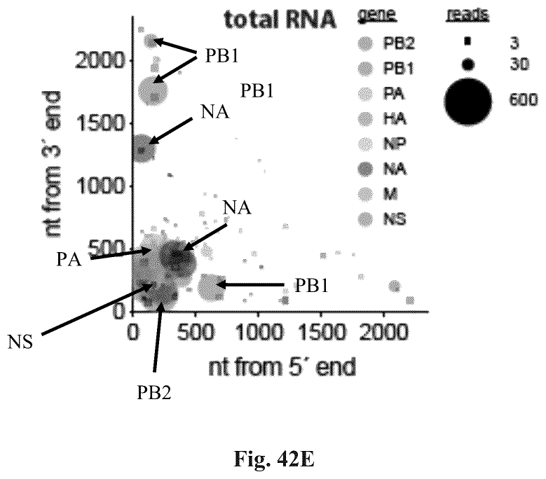

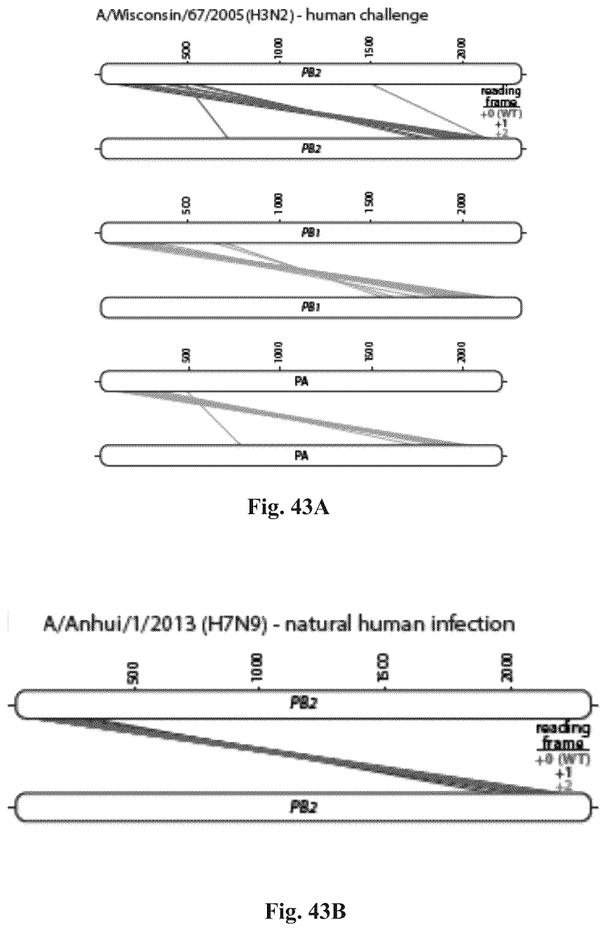

Coding potential is suggestive that DelVGs may produce proteins. To demonstrate that DelVG mRNAs are translated, we used DelVG-aware mapping to re-analyze our ribosome profiling (Ribo-Seq) data³. Ribosome-protected fragments containing DelVG junctions would indicate translation of a DelVG-encoded protein. Sequencing total RNA from infected cells showed high read coverage at the termini of PB2, consistent with the presence of DelVGs in our viral stocks (FIG. 37C). Mapping of ribosome-protected fragments on PB2 identified 50 unique DelVGs (FIG. 37C, bottom). A total of 302 unique DelVGs derived from all 8 segments was detected in ribosome-protected fragments across two biological replicates. A similar distribution of DelVGs was present in the total RNA extracted from these cells (FIG. 42B). The reading frame downstream of the deletion junctions in ribosome-protected fragments did not differ significantly from an equal distribution across all three frames (x² p>0.05). Most predicted proteins were less than 250 amino acids (FIG. 42C). Reading frame usage showed no obvious correlation with deletion size or the position of deletion junctions for both ribosome-protected fragments and total (FIG. 37D, FIG. 42B). Like the viral stocks, deletion junctions were enriched near the termini of gene segments (FIG. 37E, FIGS. 42D and 43E). Read depth of PB2 DelVGs in total RNA versus ribosome-protected fragments are correlated (FIG. 37F), suggesting that the probability of translation is a function of abundance as opposed to preference for a particular type of DelVG mRNA.

Analysis of sequencing results from human infections revealed DelVGs with protein-coding potential in human challenge studies with A/Wisconsin/67/2005(H3N2) and naturally acquired infections with A/Anhui/1/2013 (H7N9)^{4,5}. DelVGs from patients were enriched in the polymerase segments. Patient-derived PB2 DelVGs coded for protein products in all three frames (FIG. 43). Several of the PB2 DelVG-encoded proteins were very similar to those detected in our WSN stocks. Thus, DelVGs with protein coding potential arise frequently in vitro and in vivo and their mRNAs are translated into proteins, some of which contain entirely new amino acid sequences on the C-terminus derived from alternative reading frames.

### DelVG-Encoded Proteins Arise De Novo

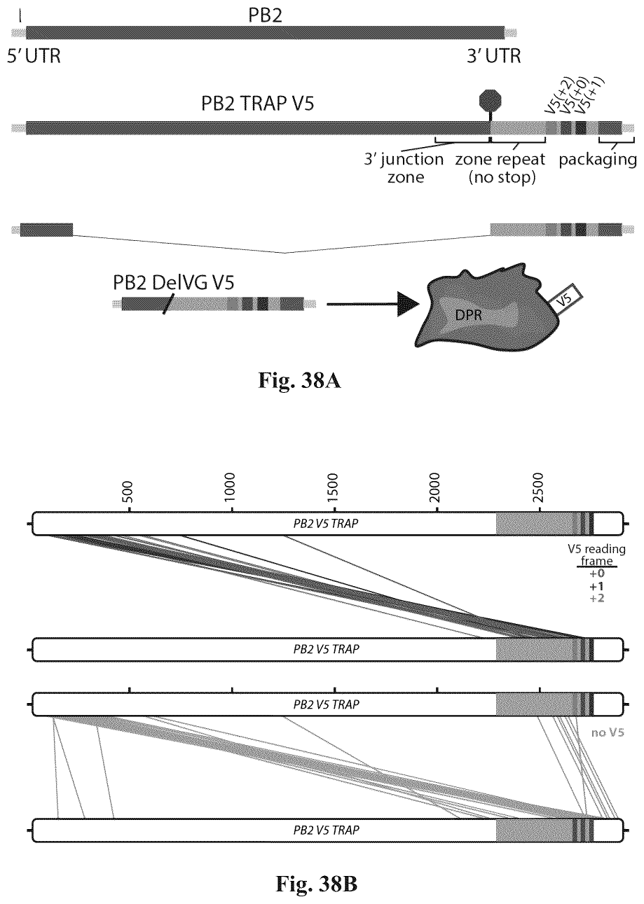

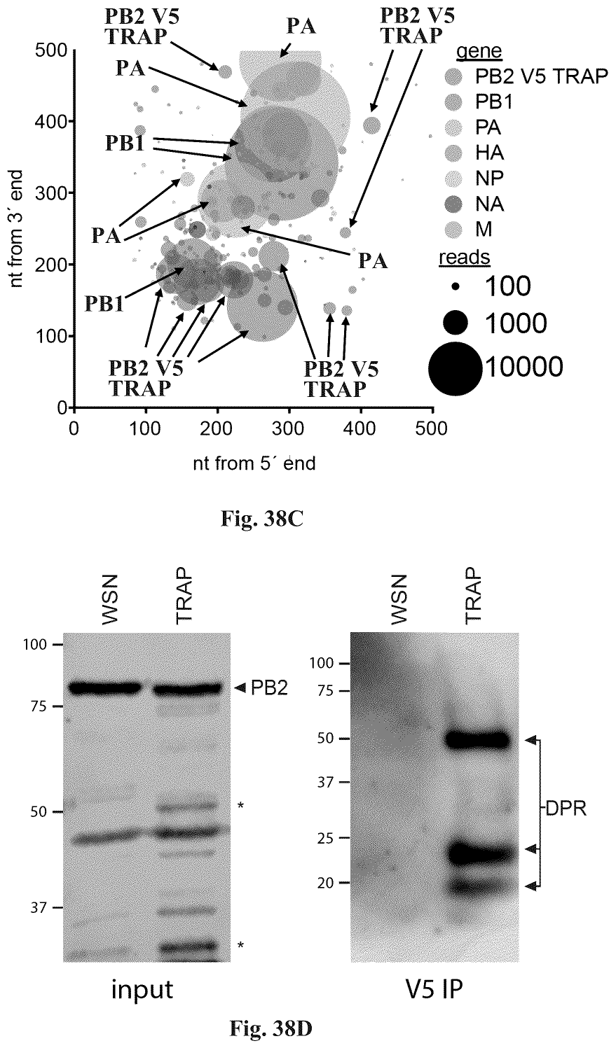

Viral stocks were produced via plasmid-based reverse genetics⁶, and start as a clonal population containing full-length genomes. To elucidate the emergence and translation of DelVGs and the potential function of these proteins, the influenza virus PB2 V5 TRAP reporter was designed (FIG. 38A). The majority of PB2 DelVGs contain 3′ junctions within the last 300 nt of the cRNA. An additional copy of this 3′ junction zone was inserted downstream of the native PB2 stop codon. The repeat was modified to remove stop codons and encode V5 epitope tags in all three frames. In this way, native PB2 is produced from full-length genomes to support viral replication, while V5-tagged proteins are only produced if deletions arise that create junctions in the repeated junction zone and maintain an open reading frame with one of the V5 coding sequences.

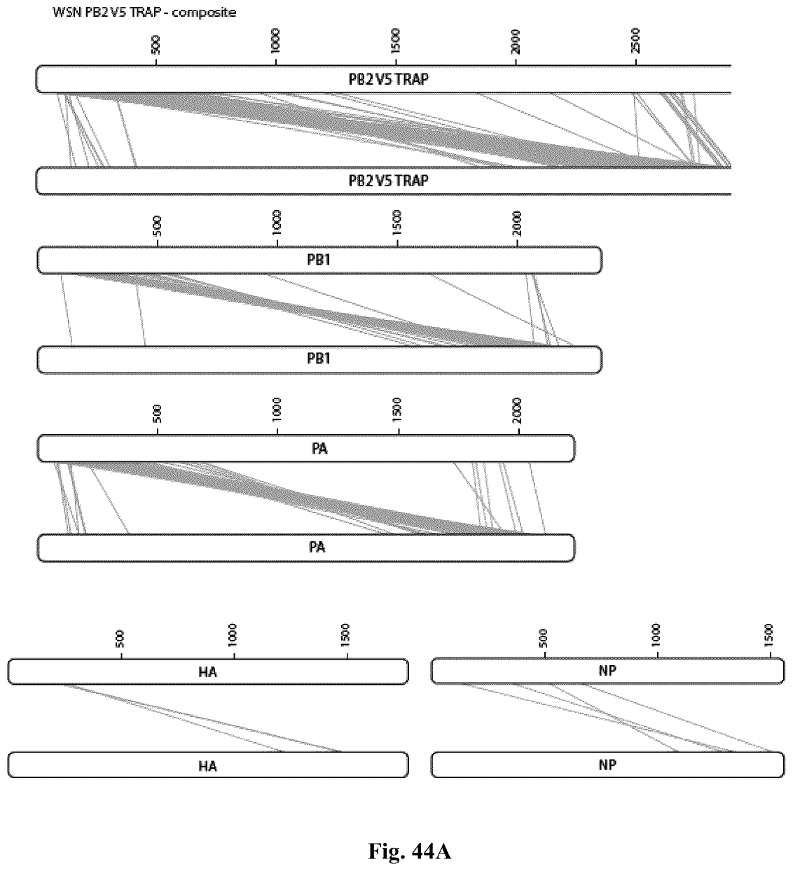

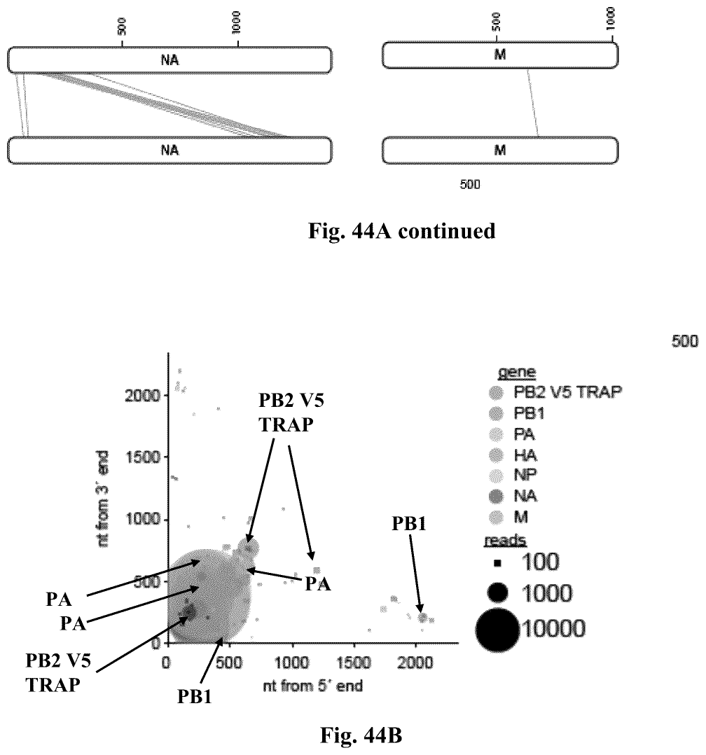

Recombinant PB2 V5 TRAP virus was rescued and used to generate plaque-purified stocks to ensure we began with a clonal population. Three clones of reporter virus were amplified and virion-associated RNA was subject to sequencing and DelVG analysis. Over 350 distinct DelVGs were detected in all segments except NS (FIG. 44A). Again, DelVGs were enriched in the polymerase genes. Patterns of DelVG junctions paralleled those present in WT WSN stocks, with the exception of the PB2 V5 TRAP reporter (FIGS. 38B and 38C). 3′ junctions in the PB2 V5 TRAP gene were relocated downstream to the repeated junction zone as anticipated (FIG. 38B). DelVGs encoding over 100 potentially V5-tagged proteins were identified with an overabundance for the +1 reading frame (x²<0.05). Whether this bias for the +1 frame represents a preference in DelVG production or is a consequence of the reporter design is not clear. Cells were infected with WT or the PB2 V5 TRAP virus. Lysates were probed for PB2, revealing multiple lower molecular proteins in the reporter virus that were not present in WT WSN (FIG. 38B). To confirm that these were DelVG-encoded, antibodies that recognize the V5 tag were used to immunopurify the lysates and blot the recovered proteins. Three prominent bands were detected exclusively in cells infected with the reporter virus, including a 50 kDa protein that was also detected in the total lysate (FIG. 38B). Whereas individual proteins produced by DelVGs had been identified previously^{7,8}, our sequence analysis, proteomics and experimental validation reveal a new class of viral proteins we refer as DPRs (DelVG-encoded proteins).

### PB2 DPRs are Dominant Negative Inhibitors of Polymerase Assembly and Viral Replication

To ascertain the function of DPRs, PB2 DPRs was cloned from the reporter virus to test their impact on polymerase activity and viral replication. Of the nine randomly cloned PB2 DelVGs, 8 have protein coding potential while PB2 32/2143 has a premature stop after only 4 codons (FIG. 39A). 5 of these potential proteins have equivalent versions when mapped back to WT PB2. As PB2 is an essential subunit of the viral polymerase, we assessed the impact of PB2 DPRs on polymerase function. PB2 DPRs were co-expressed in polymerase activity assays. Polymerase activity was significantly reduced by all of the DPRs, except PB2 32/2143, which contains a premature stop codon (FIG. 39B). Thus, proteins encoded by PB2 DelVGs can interfere with polymerase activity.

The cumulative effects of both the DelVG RNA and DPR was tested by expressing PB2 416/2189 as a minus-sense vRNA. PB2 416/2189 was the primary focus because it is the most abundant DPR in the reporter stock of all of those that were cloned and similar DelVGs were identified in the WSN stock and Ribo-seq data. The PB2 416/2189 vRNA dramatically reduced polymerase activity compared to the control (FIG. 39C). This inhibitory activity was partially dependent on the encoded DPR, as mutant containing premature stop codons had significantly reduced potency. Curiously, despite three stop codons in different frames at the very beginning of the open reading frame, this mutant still expressed low amounts of proteins, possibly by reinitiating at the methionine at position 11. While DelVGs were known to interfere with replication, combined these data now show that DPRs also contribute to inhibition.

The sequence for WT PB2 416/2189 is highly conserved, raising the possibility that DPRs from primary isolates may also have inhibitory activity (FIG. 39D). Polymerase activity assays were performed with the polymerase and NP from WSN and DPRs cloned from primary strains. We detected heterotypic inhibition of WSN polymerase using DPRs derived from the 1918 H1N1 pandemic (A/Brevig Mission/1/1918), the 2009 H1N1 pandemic (A/California/04/2009), and the emerging H7N9 virus (A/Anhui/1/2013) (FIG. 39D). Several of the potential DPRs identified in human samples were similar to the inhibitory DPRs we tested from WSN, suggesting that inhibitory activity of DPRs may be shared across influenza virus strains.

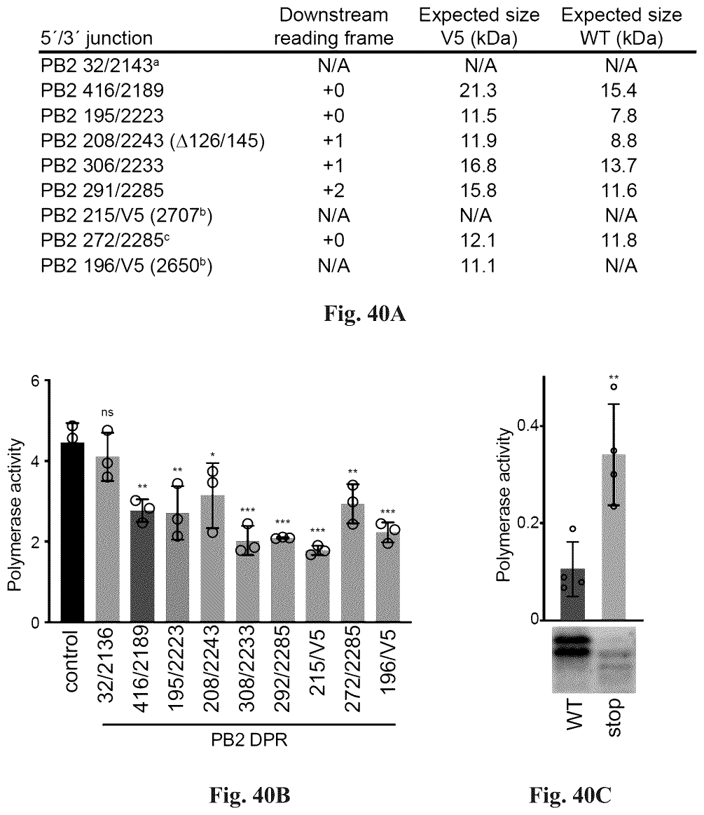

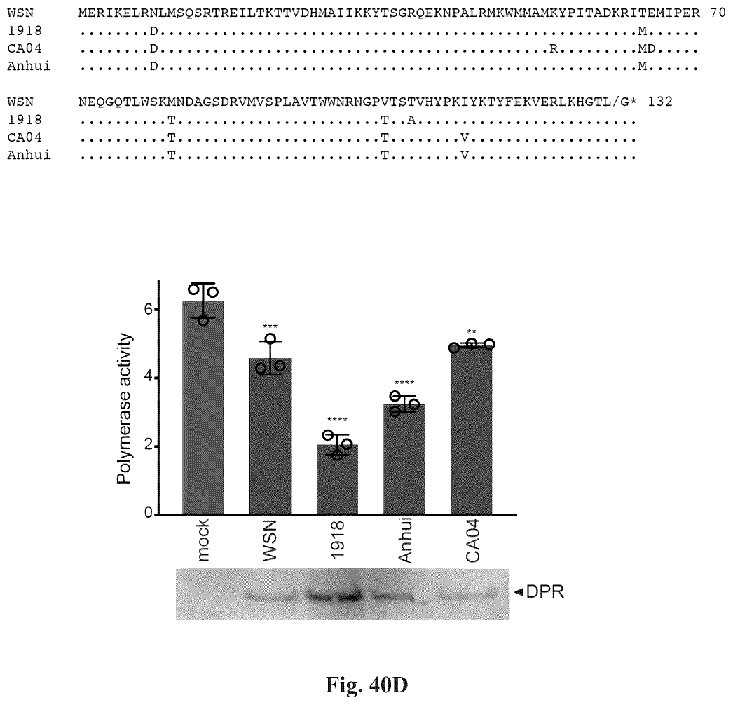

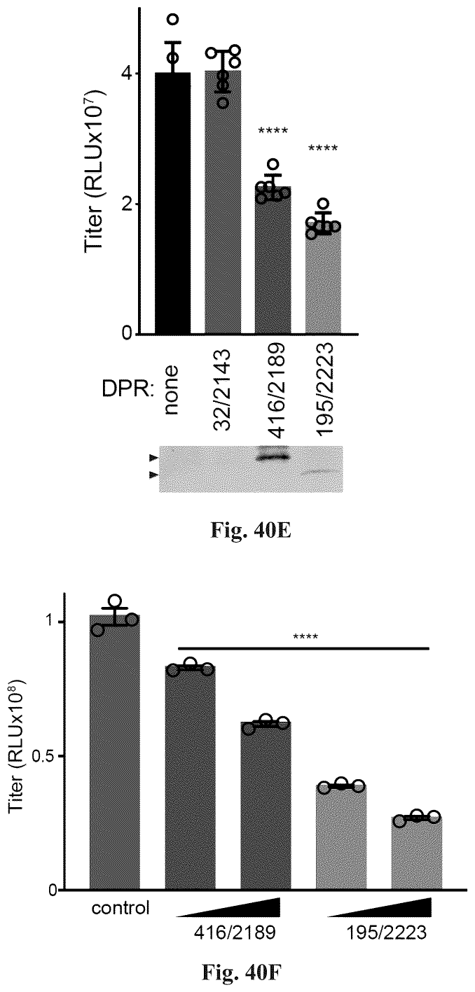

Given the ability of DPRs to disrupt polymerase activity, their impact on viral replication was tested. DPRs were expressed in cells prior to infection. Pre-expressing PB2 416/2189 or PB2 195/2223 reduced virus titers by 50% (FIG. 39E). In contrast, expressing the PB2 32/2143 that contains a premature stop codon had no effect, yielding viral titers indistinguishable from controls. To exclude the possibility that the pre-expression process artifactually altered replication, we utilized influenza virus itself to deliver the DPR. We created clonal viruses encoding PB2 416/2189 or PB2 195/2223 in place of full-length PB2. These viruses were propagated and titered on cells constitutively expressing native PB2 protein DPR viruses were used for co-infection experiments with a WT WSN NanoLuc reporter virus⁹. Titering samples using the NanoLuc reporter allowed us to uniquely measure replication of the WT virus, and not the DPR virus¹⁰. Co-infection with increasing amounts of the DPR virus showed a dose-dependent inhibition of WT WSN, reducing titers by up to 75% (FIG. 40F). Our data show that DPRs inhibit replication and viruses encoding DRPs function as classic defective interfering particles.

### PB2 DPRs are Dominant Negative Inhibitors of Polymerase Assembly

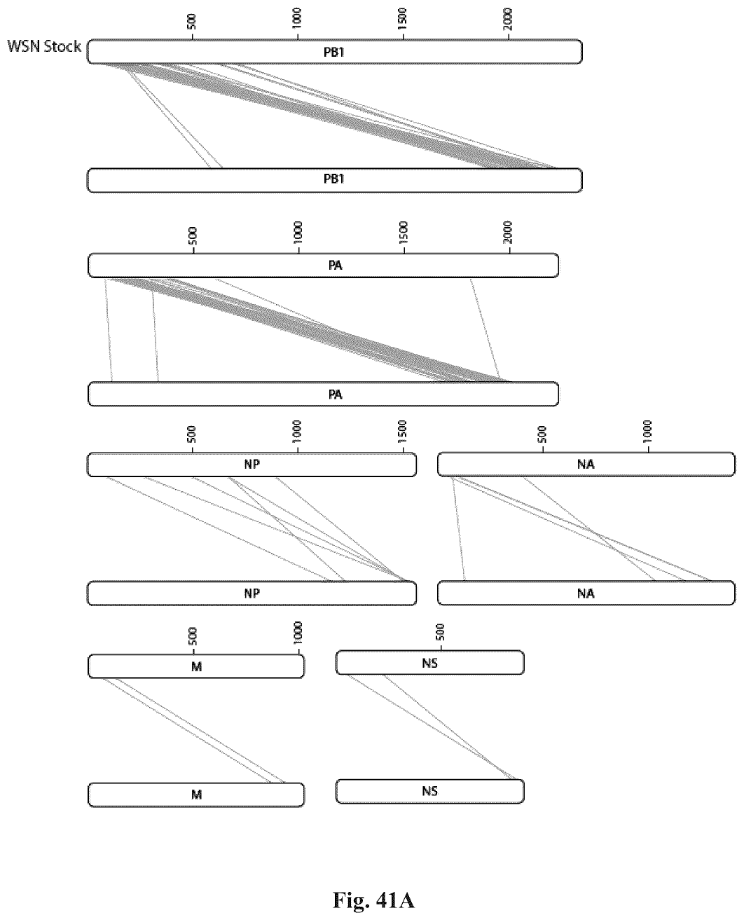

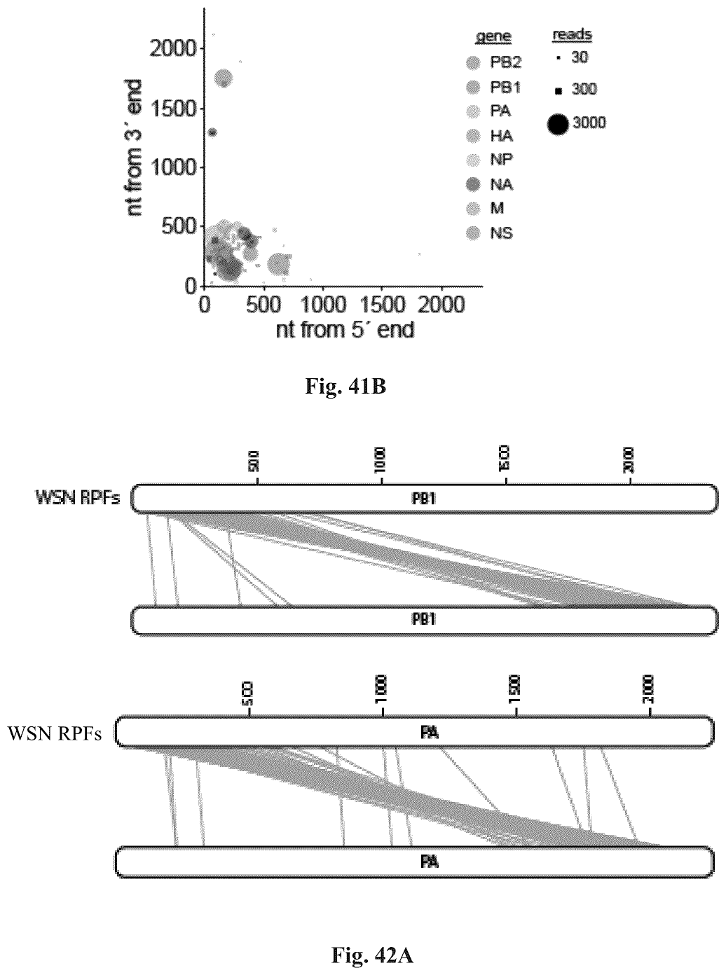

The influenza virus polymerase assembles into a functional trimer containing PB1, PB2 and PA. The N-terminal 37 amino acids of PB2 form 3 a-helices that intertwine with the C-terminus to PB111. Most the PB2 DPRs we identified retained this portion of the full-length protein. This region is sufficient for PB1 binding^{11,12}, raising the possibility that PB2 DPRs might interact with PB1. To test this, cells were infected with the PB2 V5 TRAP virus and DPRs were immunopurified from cell lysates. Blotting revealed co-precipitation of PB1 when V5-tagged DPRs were present, but not during infection with WT virus (FIG. 41A). Interactions between PB1 and PB2 DPRs were investigated further by expressing only these two proteins in cells. Again, PB1 co-precipitated with the PB2 DPRs 416/2189 and 195/2223 (FIG. 40B). Thus, not only do these proteins interact, PB2 DPRs are likely directly engaging PB1 without the need for other viral proteins.

Interfering with trimer assembly disrupts polymerase activity¹¹⁻¹⁴. Given that PB2 DPRs bind PB1 and impair polymerase function, a potential mechanism is that they competitively inhibit RNP formation. We tested this with an RNP assembly assay where the polymerase proteins and NP are expressed in cells, immunopurified via PA, and blotted to PB2. PA does not form a stable, directly interaction with PB2, therefore co-precipitated PB2 must be part of a trimeric complex and reflect successful polymerase assembly. PB2 is readily co-precipitated with PA under control conditions (FIG. 40C). However, expressing increasing amount of PB2 416/2189 displaced WT PB2, and instead the DPR was co-precipitated. These data demonstrate that PB2 DPRs are dominant negative inhibitors of trimeric polymerase assembly, causing reduced polymerase activity and viral replication.

### REFERENCES

1. Alnaji, F. G., Holmes, J. R., Rendon, G., Vera, J. C., Fields, C. J., Martin, B. E., and Brooke, C. B. (2019). Sequencing Framework for the Sensitive Detection and Precise Mapping of Defective Interfering Particle-Associated Deletions across Influenza A and B Viruses. Journal of Virology 93, e00354-19. 10.1128/jvi.00354-19.
2. Mendes, M., and Russell, A. B. (2021). Library-based analysis reveals segment and length dependent characteristics of defective influenza genomes. PLOS Pathogens 17, e1010125. 10.1371/journal.ppat. 1010125.
3. Tran, V., Ledwith, M. P., Thamamongood, T., Higgins, C. A., Tripathi, S., Chang, M. W., Benner, C., García-Sastre, A., Schwemmle, M., Boon, A. C. M., et al. (2020). Influenza virus repurposes the antiviral protein IFIT2 to promote translation of viral mRNAs. Nature Microbiology 5, 1490-1503. 10.1038/s41564-020-0778-x.
4. Lui, W.-Y., Yuen, C.-K., Li, C., Wong, W. M., Lui, P.-Y., Lin, C.-H., Chan, K.-H., Zhao, H., Chen, H., To, K. K. W., et al. (2019). SMRT sequencing revealed the diversity and characteristics of defective interfering RNAs in influenza A (H7N9) virus infection. Emerging Microbes & Infections 8, 662-674. 10.1080/22221751.2019.1611346.
5. Martin, M. A., Kaul, D., Tan, G. S., Woods, C. W., and Koelle, K. (2019). The Dynamics of Influenza A H3N2 Defective Viral Genomes from a Human Challenge Study. 814673. 10.1101/814673.
6. Neumann, G., Fujii, K., Kino, Y., and Kawaoka, Y. (2005). An improved reverse genetics system for influenza A virus generation and its implications for vaccine production. Proceedings of the National Academy of Sciences of the United States of America 102, 16825-16829. 10.1073/pnas.0505587102.
7. Akkina, R. K., Chambers, T. M., and Nayak, D. P. (1984). Expression of defective-interfering influenza virus-specific transcripts and polypeptides in infected cells. Journal of Virology. 10.1128/jvi.51.2.395-403.1984.
8. Boergeling, Y., Rozhdestvensky, T. S., Schmolke, M., Resa-Infante, P., Robeck, T., Randau, G., Wolff, T., Gabriel, G., Brosius, J., and Ludwig, S. (2015). Evidence for a Novel Mechanism of Influenza Virus-Induced Type I Interferon Expression by a Defective RNA-Encoded Protein. PLOS Pathogens 11, e1004924. 10.1371/journal.ppat. 1004924.
9. Tran, V., Moser, L. A., Poole, D. S., and Mehle, A. (2013). Highly sensitive real-time in vivo imaging of an influenza reporter virus reveals dynamics of replication and spread. Journal of Virology 87, 13321-13329. 10.1128/JVI.02381-13.
10. Karlsson, E. A., Meliopoulos, V. A., Tran, V., Savage, C., Livingston, B., Schultz-Cherry, S., and Mehle, A. (2018). Measuring Influenza Virus Infection Using Bioluminescent Reporter Viruses for In Vivo Imaging and In Vitro Replication Assays. In Methods in Molecular Biology, pp. 431-459. 10.1007/978-1-4939-8678-1_21.
11. Sugiyama, K., Obayashi, E., Kawaguchi, A., Suzuki, Y., Tame, J. R. H., Nagata, K., and Park, S. Y. (2009). Structural insight into the essential PB1-PB2 subunit contact of the influenza virus RNA polymerase. EMBO Journal 28, 1803-1811. Doi 10.1038/Emboj.2009.138.
12. Reuther, P., Manz, B., Brunotte, L., Schwemmle, M., and Wunderlich, K. (2011). Targeting of the influenza A virus polymerase PB1-PB2 interface indicates strain-specific assembly differences. Journal of Virology 85, 13298-13309. 10.1128/JVI.00868-11.
13. Wunderlich, K., Mayer, D., Ranadheera, C., Holler, A. S., Manz, B., Martin, A., Chase, G., Tegge, W., Frank, R., Kessler, U., et al. (2009). Identification of a PA-binding peptide with inhibitory activity against influenza A and B virus replication. PloS One 4, e7517. 10.1371/journal.pone.0007517.
14. Wunderlich, K., Juozapaitis, M., Ranadheera, C., Kessler, U., Martin, A., Eisel, J., Beutling, U., Frank, R., and Schwemmle, M. (2011). Identification of high-affinity PB1-derived peptides with enhanced affinity to the PA protein of influenza A virus polymerase. Antimicrobial Agents and Chemotherapy 55, 696-702. 10.1128/AAC.01419-10.
15. Lee, J. M., Huddleston, J., Doud, M. B., Hooper, K. A., Wu, N. C., Bedford, T., and Bloom, J. D. (2018). Deep mutational scanning of hemagglutinin helps predict evolutionary fates of human H3N2 influenza variants. Proceedings of the National Academy of Sciences of the United States of America 115, E8276-E8285 10.1073/pnas. 1806133115.
16. Mehle, A., and Doudna, J. A. (2008). An inhibitory activity in human cells restricts the function of an avian-like influenza virus polymerase. Cell Host and Microbe 4, 111-122. 10.1016/j.chom.2008.06.007.
17. Dos Santos Afonso, E., Escriou, N., Leclercq, I., van der Werf, S., and Naffakh, N. (2005). The generation of recombinant influenza A viruses expressing a PB2 fusion protein requires the conservation of a packaging signal overlapping the coding and noncoding regions at the 5′ end of the PB2 segment. Virology 341, 34-46. 10.1016/j.virol.2005.06.040.
18. Mehle, A., and Doudna, J. A. (2009). Adaptive strategies of the influenza virus polymerase for replication in humans. Proceedings of the National Academy of Sciences of the United States of America 106, 21312-21316. 10.1073/pnas.0911915106.
19. Tran, V., Poole, D. S., Jeffery, J. J., Sheahan, T. P., Creech, D., Yevtodiyenko, A., Peat, A. J., Francis, K. P., You, S., and Mehle, A. (2015). Multi-Modal Imaging with a Toolbox of Influenza A Reporter Viruses. Viruses 7, 5319-5327. 10.3390/v7102873.
20. Routh, A., and Johnson, J. E. (2014). Discovery of functional genomic motifs in viruses with ViReMa-a Virus Recombination Mapper—for analysis of next-generation sequencing data. Nucleic Acids Res 42, e11. 10.1093/nar/gkt916.
21. Li, H. (2018). Minimap2: pairwise alignment for nucleotide sequences. Bioinformatics 34, 3094-3100. 10.1093/bioinformatics/bty 191.
22. Wickham, H (2016). ggplot2: Elegant Graphics for Data Analysis (Springer-Verlag). https://ggplot2.tidyverse.org
23. R Core Team (2021). R: A Language and Environment for Statistical Computing (Vienna Austria).
24. Rice, P., Longden, I., and Bleasby, A. (2000). EMBOSS: the European Molecular Biology Open Software Suite. Trends Genet 16, 276-277. 10.1016/s0168-9525(00)02024-2.
25. Larson, G. P., Tran, V., Yú, S., Cai, Y., Higgins, C. A., Smith, D. M., Baker, S. F., Radoshitzky, S. R., Kuhn, J. H., and Mehle, A. (2019). EPS8 Facilitates Uncoating of Influenza A Virus. Cell Reports 29, 2175-2183. 10.1016/j.celrep.2019.10.064.
26. Marsh, G. A., Hatami, R., and Palese, P. (2007). Specific residues of the influenza A virus hemagglutinin viral RNA are important for efficient packaging into budding virions. Journal of Virology 81, 9727-9736. 10.1128/JVI.01144-07.

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

1. An isolated influenza virus comprising:
i) 8 different gene segments including a PA viral gene segment, a PB1 viral gene segment, a mutant PB2 viral gene segment, a HA viral gene segment, a NA viral gene segment, a NP viral gene segment, a M (M1 and M2) viral gene segment, and a NS (NS1 and NS2) viral gene segment, or
ii) 8 different gene segments including a PA viral gene segment, a PB1 viral gene segment, a mutant PB2 viral gene segment, a HA viral gene segment, a NA (NA and NB) viral gene segment, a NP viral gene segment, a M (M1 and BM2) viral gene segment and a NS (NS1 and NS2) viral gene segment;
wherein the mutant PB2 viral gene segment includes 5′ and 3′ packaging sequences including 3′ or 5′ coding and non-coding packaging sequences flanking a nucleotide sequence having PB2 sequences that encode an inhibitory protein or a nucleotide sequence having PB2 sequences that encode a polypeptide having at least 5, 10, 15, 20, 25, 30, 35 or 39, or any integer between 1 and 40 N-terminal amino acids of PB2, wherein the mutant PB2 segment does not include contiguous sequences corresponding to sequences encoding a functional PB2.

2. The isolated influenza virus of claim 1 wherein the encoded protein comprises any one of SEQ ID Nos. 1 to 44 or a protein having at least 80%, 85%, 90%, 92%, 95%, 97% or 99% amino acid sequence identity thereto.

3. The isolated influenza virus of claim 1 which is an influenza A virus.

4. The isolated influenza virus of claim 1 which is an influenza B virus.

5. The isolated influenza virus of claim 2 which is an influenza A virus.

6. The isolated influenza virus of claim 2 which is an influenza B virus.

7. A composition comprising the isolated influenza virus of claim 1 which optionally comprises replication competent influenza virus.

8. An isolated nucleic acid comprising a nucleotide sequence encoding any one of SEQ ID Nos. 1 to 44, or a protein having at least 80%, 85%, 90%, 92%, 95%, 97% or 99% amino acid sequence identity thereto.

9. The isolated nucleic acid of claim 8, further comprising a promoter operably linked to the nucleotide sequence.

10. The isolated nucleic acid of claim 9, wherein the promotor comprises a nucleic acid sequence of any one of SEQ ID Nos. 46, 47, 49, or 58.

11. The isolated nucleic acid of claim 8, which is a viral vector.

12. The isolated nucleic acid of claim 8, further comprising lipids or a polymer.

13. A composition comprising the isolated nucleic acid of claim 8.

14-15. (canceled)

16. A nanoparticle or microparticle comprising the isolated nucleic acid of claim 8.

17. The nanoparticle or microparticle of claim 16 which comprises a liposome.

18. The nanoparticle or microparticle of claim 16 which comprises a synthetic or natural polymer.

19-27. (canceled)

28. A method to prevent, inhibit or treat an influenza virus infection in a vertebrate, comprising: contacting the vertebrate with the composition of claim 13.

29. The method of claim 28 wherein the vertebrate is an avian or a mammal.

30. The method of claim 29 wherein the mammal is a human.

31. The method of claim 28 wherein the composition further comprises an influenza virus.

32. The method of claim 28 wherein the composition is intramuscularly, intranasally, systemically, or subcutaneously administered.

33-44. (canceled)


SEQUENCE LISTING (71 sequences)

SEQ ID NO: 1 - protein, 40 aa, synthetic construct
MERIKELRNLMSQSRTREILTKTTVDHTQMKAQLELSPQF

SEQ ID NO: 2 - protein, 76 aa, synthetic construct
MERIKELRNLMSQSRTREILTKTTVDHMAIIKKYTSGRQEKNPALRMKWMMAMKYPITAANWARRRGVGNETETEL

SEQ ID NO: 3 - protein, 58 aa, synthetic construct
MERIKELRNLMSQSRTREILTKTTVDHMAIIKKYTSGRQEKNPALRMKWMMAMKYPIK

SEQ ID NO: 4 - protein, 93 aa, synthetic construct
MERIKELRNLMSQSRTREILTKTTVDHMAIIKKYTSGRQEKNPALRMKWMMAMKYPITADKRITEMLVMKRKRNSSILTDSQTATKRIRMAIN

SEQ ID NO: 5 - protein, 79 aa, synthetic construct
MERIKELRNLMSQSRTREILTKTTVDHMAIIKKYTSGRQEKNPALRMKWMMAMKYPITADKRITEMIPERNEQGQTLMC

SEQ ID NO: 6 - protein, 57 aa, synthetic construct
MERIKELRNLMSQSRTREILTKTTVDHMAIIKKYTSGRQEKNPALRMKWMMAMMLAL

SEQ ID NO: 7 - protein, 134 aa, synthetic construct
MERIKELRNLMSQSRTREILTKTTVDHMAIIKKYTSGRQEKNPALRMKWMMAMKYEGTAGVESAVLRGFLILGKEDRRYGPALSINELSNLAKGEKANVLIGQGDVVLVMKRKRNSSILTDSQTATKRIRMAIN

SEQ ID NO: 8 - protein, 92 aa, synthetic construct
MERIKELRNLMSQSRTREILTKTTVDHMAIIKKYTSGRQEKNPALRMKWMMAMKYPITADKRITEMIPERNEQGQTLWSKMNDEQPCERREG

SEQ ID NO: 9 - protein, 76 aa, synthetic construct
MERIKELRNLMSQSRTREILTKTTVDHMAIIKKYTSGRQEKNPALRMKWMMAMKYPITADKRIRRRGVGNETETEL

SEQ ID NO: 10 - protein, 31 aa, synthetic construct
MERIKELRNLMSQSRTREILTKTTVERMLAL

SEQ ID NO: 11 - protein, 48 aa, synthetic construct
MERIKELRNLMSQSRTREILTKTTVDHMAIIKKYTSGRQEIWTSIKHK

SEQ ID NO: 12 - protein, 56 aa, synthetic construct
MERIKELRNLMSQSRTREILTKTTVDHMAIIKKYTSGRQEKNSSFWAKKTGDMDQH

SEQ ID NO: 13 - protein, 48 aa, synthetic construct
MERIKELRNLMSQSRTREILTKTTVDHMAIIKKYTSGRQEKNPALKHK

SEQ ID NO: 14 - protein, 93 aa, synthetic construct
MERIKELRNLMSQSRTREILTKTTVDHMAIIKKYTSGRQEKNPALRMKWMMAMKYPITADKRITEMIPERNEQGQTLWSKMNDAARKGCWPFN

SEQ ID NO: 15 - protein, 33 aa, synthetic construct
MERIKELRNLMSQSRTREILTKTTVDHMAIIKK

SEQ ID NO: 16 - protein, 96 aa, synthetic construct
MERIKELRNLMSQSRTREILTKTTVDHMAIIKKYTSGRQEKNPALSINELSNLAKGEKANVLIGQGDVVLVMKRKRNSSILTDSQTATKRIRMAIN

SEQ ID NO: 17 - protein, 94 aa, synthetic construct
MERIKELRNLMSQSRTREILTKTTVDHMAIIKKYTSGRQEKNPALRMKWMMAMKYPITADKRITEMIPERNEQGQTLWSKMNDAGSSIQLQQDH

SEQ ID NO: 18 - protein, 74 aa, synthetic construct
MERIKELRNLMSQSRTREILTKTTVDHMAIIKKYTSGRQEKNPALRMKWMMAMKYPITADKRITEMIPERRLMC

SEQ ID NO: 19 - protein, 27 aa, synthetic construct
MERIKELRNLMSQSRTREILTKTTVAL

SEQ ID NO: 20 - protein, 185 aa, synthetic construct
MERIKELRNLMSQSRTREILTKTTVDHMAIIKKYTSGRQEKNPALRMKWMMAMKYPITADKRITEMIPERNEQGQTLWSKMNDAGSDRVMVSPLAVTWWNRNGPVTSTVHYPKIYKTYFEKVERLKHGTFGPVHFRNQVKILAKGEKANVLIGQGDVVLVMKRKRNSSILTDSQTATKRIRMAIN

SEQ ID NO: 21 - protein, 110 aa, synthetic construct
MERIKELRNLMSQSRTREILTKTTVDHEDPDEGTAGVESAVLRGFLILGKEDRRYGPALSINELSNLAKGEKANVLIGQGDVVLVMKRKRNSSILTDSQTATKRIRMAIN

SEQ ID NO: 22 - protein, 111 aa, synthetic construct
MERIKELRNLMSQSRTREILTKTTVDHMAIIKKYTSGRQEKNPALRMKWMMAMKYPITADKRITEMIPERNEQGQTLWSKMNDAGSDRVMVSPLFPSQPLHQSKVERSSPH

SEQ ID NO: 23 - protein, 80 aa, synthetic construct
MERIKELRNLMSQSRTREILTKTTVDHMAIIKKYTSGRQEKNPALRMKWMMAMKYPITADKRITEMIPERNEQPCERREG

SEQ ID NO: 24 - protein, 35 aa, synthetic construct
MERIKELRNLMSQSRTREILTKTTVDHMAIIKKYT

SEQ ID NO: 25 - protein, 222 aa, synthetic construct
MERIKELRNLMSQSRTREILTKTTVDHMAIIKKYTSGRQEKNPALRMKWMMAMKYPITADKRITEMIPERNEQGQTLWSKMNDAGSDRVMVSPLAVTWWNINVRGSGMRILVRGNSPVFNYNKTTKRLTVLGKDAGPLTEDPDEGTAGVESAVLRGFLILGKEDRRYGPALSINELSNLAKGEKANVLIGQGDVVLVMKRKRNSSILTDSQTATKRIRMAIN

SEQ ID NO: 26 - protein, 48 aa, synthetic construct
MERIKELRNLMSQSRTREILTKTTVDHMAIIKKYTSGRQEKNPALRMK

SEQ ID NO: 27 - protein, 59 aa, synthetic construct
MERIKELRNLMSQSRTREILTKTTVDHMAIIKKYTSGRQEKNPALRMKWTEQPCERREG

SEQ ID NO: 28 - protein, 146 aa, synthetic construct
MERIKELRNLMSQSRTREILTKTTVDHMAIIKKYTSGRQEKNPALRMKWMMAMKYPITADKRITEMIPERNEQGQTLWSKMNDAGSDRVMVSPLAVTNELSNLAKGEKANVLIGQGDVVLVMKRKRNSSILTDSQTATKRIRMAIN

SEQ ID NO: 29 - protein, 93 aa, synthetic construct
MERIKELRNLMSQSRTREILTKTTVDHMAIIKKYTSGRQEKNPALRMKWMMAMKYPITADKRITEVLVMKRKRNSSILTDSQTATKRIRMAIN

SEQ ID NO: 30 - protein, 114 aa, synthetic construct
MERIKELRNLMSQSRTREILTVLGKDAGPLTEDPDEGTAGVESAVLRGFLILGKEDRRYGPALSINELSNLAKGEKANVLIGQGDVVLVMKRKRNSSILTDSQTATKRIRMAIN

SEQ ID NO: 31 - protein, 179 aa, synthetic construct
MERIKELRNLMSQSRTREILTKTTVDHMAIIKKYTSGRQEKNPALRMKWMMAMKYPIINVRGSGMRILVRGNSPVFNYNKTTKRLTVLGKDAGPLTEDPDEGTAGVESAVLRGFLILGKEDRRYGPALSINELSNLAKGEKANVLIGQGDVVLVMKRKRNSSILTDSQTATKRIRMAIN

SEQ ID NO: 32 - protein, 68 aa, synthetic construct
MERIKELRNLMSQSRTREILTKTTVDHMAIIKKYTSGRQEKNPALRMKWMMAMKYPITADKRITETEL

SEQ ID NO: 33 - protein, 114 aa, synthetic construct
MERIKELRNLMSQSRTREILTKTTVDHMAIIKKYTSGRQEKNPALRMKWMMAMKEDRRYGPALSINELSNLAKGEKANVLIGQGDVVLVMKRKRNSSILTDSQTATKRIRMAIN

SEQ ID NO: 34 - protein, 60 aa, synthetic construct
MERIKELRNLMSQSRTREILTKTTVDHMAIIKKYTSGRQEKNPALRMKWMSSRKGCWPFN

SEQ ID NO: 35 - protein, 137 aa, synthetic construct
MERIKELRNLMSQSRTREILTKTTVDHMAIIKKYTSGRQEKNPALRMKWMMAMKYPITADKRITEMIPERNEQGQTLWSKMNDAGSDREIGWPSRKAYPKPSPRSRFYEVSPSLTRSSASIPRGKPIPNPLLGLDST

SEQ ID NO: 36 - protein, 191 aa, synthetic construct
MERIKELRNLMSQSRTREILTKTTVDHMAIIKKYTSGRQEKNPALRMKWMMAMKYPITADKRITEMIPERNEQGQTLWSKMNDAGSDRVMVSPLAVTWWNRNGPVTSTVHYPKIYKTYFEKVERLKHGTLGKCANWARRRGVGKETETELKHTYRQPDSDQKNSDGHQLVSNSLKTTLFLLGPYSIVSPKC

SEQ ID NO: 37 - protein, 98 aa, synthetic construct
MERIKELRNLMSQSRTREILTKTTVDHMAIIKKYTSGRQEKNPALRMKWMMAMKYPVGKETETELKHTYRQPDSDQKKSDGHQGKPIPNPLLGLDSTR

SEQ ID NO: 38 - protein, 104 aa, synthetic construct
MERIKELRNLMSQSRTREILTKTTVDHMAIIKKEKNPALRMKWMMAMKYPITADRNSSILTDSQTATKRNRMAIKESLSQTLSSVSILRGKPIPNPLLGLDSTR

SEQ ID NO: 39 - protein, 146 aa, synthetic construct
MERIKELRNLMSQSRTREILTKTTVDHMAIIKKYTSGRQEKNPALRMKWMMAMKYPITADKRITEMIPERNEQGQTLWSKMNDAGSDRVMVSPKRKRNSSILTDSQTATKRNRMAIKESLSQTLSSVSILRGKPIPNPLLGLDSTR

SEQ ID NO: 40 - protein, 4 aa, synthetic construct
MDQH

SEQ ID NO: 41 - protein, 70 aa, synthetic construct
MERIKELRNLMSQSRTREILTKTTVDHMAIIKKYTSGRQEKNPALRMKWMMAMKYPITADKRILGLDSTR

SEQ ID NO: 42 - protein, 103 aa, synthetic construct
MERIKELRNLMSQSRTREILTKTTVDHMAIIKKYTSGRQEKNPALRMKWMMAMKYPITADKRITEMIPERNEQGQTLWSKMKKSDGHQGKPIPNPLLGLDSTR

SEQ ID NO: 43 - protein, 96 aa, synthetic construct
MERIKELRNLMSQSRTREILTKTTVDHMAIIKKYTSGRQEKNPALRMKWMMAMKYPNPKPSPRSRFYEVSPSLTRSSASIPRGKPIPNPLLGLDST

SEQ ID NO: 44 - protein, 76 aa, synthetic construct
MERIKELRNLMSQSRTREILTKTTVDHMAIIKKYTSGRQEKNPALRMESLSQTLSSVSILRGKPIPNPLLGLDSTR

SEQ ID NO: 45 - DNA, 12 bp, synthetic construct
  source 1..12: mol_type = other DNA
agcgaaagcagg

SEQ ID NO: 46 - DNA, 12 bp, synthetic construct
  source 1..12: mol_type = other DNA
agtggaaacagg

SEQ ID NO: 47 - protein, 12 aa, synthetic construct
AGTGAAAACAGG

SEQ ID NO: 48 - protein, 12 aa, synthetic construct
AGCAAAAGCAGG

SEQ ID NO: 49 - protein, 12 aa, synthetic construct
AGTAGAAACAGG

SEQ ID NO: 50 - RNA, 2341 nt, Influenza A
  source 1..2341: mol_type = other RNA
agtagaaacaaggtcgtttttaaactattcgacactaattgatggccatccgaattcttttggtcgctgtctggctgtcagtaagtatgctagagttccgtttccgtttcattaccaacaccacgtctccttgcccaattagcacattagccttctctcctttcgcaaggttgctcagttcatttatgcttaatgctggtccatatctcctgtcttctttgcccagaatgaggaatcctctcagaactgcggactcaactccagctgtgccttcatctgggtcttcagttaaagggccagcatcctttccgagaactgtgagtcttttagtggtcttgttgtagttgaatactggagaattgccccttacaagtattctcattcctgatcccctcacatttatagtcaatgaggagaactgcgttctactttgctttggtggagcggctgcgaagggaagaagttttattatctgagcggtatcaaatgtcccaagcacatccctcatttgttggaacagagttctcacaaacccactgtattggcctctaacggcctttggaactaaagactgaaatggctcaaattccattttattgtacagcattgtaggattctgggaccactgaattttaacagtttcccagtttctgatgatccactgataggtattgaccaacactgattcaggaccattaatctcccacatcattgacgatgagtaagttattgtcagtttctctgttccctgtgtttcactgatctcctcgggagacagtagtacattcccacgttggtccctaactctcaaaaaacggtcaatgctcaccactatcttctccgcgctggaatactcatctacccccattttgctgattctcactcctctcattgacatctcggtgcttggagtcatgtcgggcaatatcccgatcattcccatcacattgtcgatggattcaattccccaattttgaaagagcacctttgcatccttctgaaaatgtctcaaaagttggtgcatgggattcaatcgctgattcgccctattgacgaaattcaggtcacctctaactgcttttatcatacaatcctcttgtgaaaataccatggccacaattattgcttcggcaatcgactgttcgtctctcccactcactatcagctgaatcaatctcctggttgcttttctgagtatagctgttgctcttctcccaaccattgtgaactcttcatatccctcatgcactcttatcttcaatgtctgaagattgcccgtaagcacctcttcctctctcttgactgatgatccgcttgttctcttaaatgtgaatccaccaaaactgaaggatgagctaattctcagtcccattgcagccttgcaaatatccacggcttgctcttctgttgggttctgcctaaggatgtttaccatccttattccaccaatctgcgtgctgtggcacatctccaataaagatgctagtggatctgctgatactgtggctcttcttactatgtttctagcagcaataattaagctttgatcaacatcatcattcctcgcctcccctcctggagtgtacatctgttcccagcatgttccttgggtcaaatgcaacacttcaatgtacacactgcttgttccaccagccactgggaggaatctcgttttgcggaccagttctctctccaacatgtatgccaccatcagaggagaaattttgcaaccctggagttcttctttcttctctttggttgtcgttagttgcgattccgatgttagtatcctggctcccacttcgttagggaaaacaacttccatgattacatcctgtgcctctttggcactgagatctgcatgaccaggatttatgtcaactcttcgacgtattttgacttggtttctaaaatggacagggccaaaggttccatgttttaacctttcgactttttcaaaataagttttgtagatttttggataatgaactgtacttgtcactggtccattcctattccaccatgtcacagccagaggtgataccatcactcggtctgatccggcgtcattcattttactccataaagtttgtccctgctcatttctctcaggaatcatttccgttatcctcttgtctgctgtaattggatatttcattgccatcatccatttcatcctaagtgctgggttcttctcctgtcttcctgatgtgtacttcttgattatggccatatggtccacggtggtttttgtgagtatctcgcgagtgcgagactgcgacattagattccttagttcttttattctttccatattgaatataattgacctgctttcgct

SEQ ID NO: 51 - RNA, 2341 nt, Influenza A
  source 1..2341: mol_type = other RNA
agtagaaacaaggcattttttcatgaaggacaagctaaattcactatttttgccgtctgagctcttcaatggtggaacagatcttcatgatctcagtgaactcctctttctttatccttccagattcgaaatcaattcgtgcatcaattcgggctctggaaaccatagcctccaccatactggatatcccgactggtcttctgtatgaactgctggggaagaatttttcaaataagttgcagcacttttggtacatttgttcatcttcaagtattcctctttggcttgtattcaagatggatcgatttcttttggggatccaggagtgtgttgttgcaacagcatcatactccatgtttttggctggaccatgtgctggcattatcactgcattgttcactgattcaatgtctttatggttgacaaatgggttcagtgggttgcataaacgcccctggtaatcctcatccattaattcccatttcaagcagacttcaggaatgtggagatttctaatgttgtataaatttgggcctccgtcggagaccagcagtccagctttggaatgggtttgctcccacagtttctttatttcaaatgatcttcgggtttgtatttgtgtgtcacctctatggcaccggtacgtgtacctgtaatctttgatgaacagctgaagggccatttgagcggttgctggaccaagatcattgtttatcatattgtttttgatgacagtaactccaatactcatgtccgcagactcgttgatcccagacaccccaaagctgggaagctccatgctgaaattggcaacaaacccataacgatagaaaaaacttgtgaattcaaatgtacctgttctgtttatgtaagactttttcttgctcatattaattccaagtagcttacaggttcgataaaacctgttgactccggcttgaatcccttcatgattgggtgcattcacaatcagagcaaaatcatcagaagattgaagaccatcccaccagtaagtagtcttggtgtgtctcttttgtccaagattcaggatggagacgcctaatacagtacttaacatattgaacatgcccatcatcattccagggctcaatgatgcagtcccatctattaagagcggccggattttttcaatcttctttctagttgaatcattgaagtatttcaaatcgatgcttgctagcatttctgcaggtatttgagttctaattttcatactcttgctctcaaacatgtacccctttcccagtctcgccattttgtttgagaacattattggagcaatacttagaacatttctgaaccattcgggctgatttctggttatatatgtgatcatggccaaaaacatccgagggttctgattttcgttccatttggtgttatctccagtgatggtgaaagaaatttcagtgtcctgagaattggtcatcatcttccttacaacatttgccaactttgctttcttctcattgcctccaactggcaatcctgattgttcaagtttctcacatatactccttgctagtgtctcaacaaagtatacaaacccccttatttgcatccctggggttgcaattgctctccgttttagcttccctctctcagcatctttggtcattgtgttcagggttaatgccctaattagataactccttttgttcaatctctgcttccttttacctattgttctctgtgtcaccattttcttagtcatattgtctctcactcgtctctttctctgaaaatgagttgtgatctccatttcttctttgttcattgactccattacatccttaaggaagtctatgagccttccagattcattggccgtgaggccatttgatctgaacacttctattgtgttggccaatgctgttgcagcaggctggttcctatttagagtccagtcataggtctgtcggccttgtgtcagcttgtccactcgtgtttgctgaacaacctccatcgtttcaagacacgaggtctcaaagataccaggatgggattcctcaaggaaggccattgcttccaatacacaatctgtttgggcataaccacttggttcattgtcttctggcagtggcccatcaatcgggttgagttgcggtgctccagtttcggtgtttgttgtccatcttcccctttctgagtactgatgtgtcctgttgacagtatccatggtgtatcctgttcctgtcccatggctgtaaggagggtctccagtataagggaaagttgtgcttatagcattttgtgctggcacttttaagaaaagtaaagtcggattgacatccattcaaatggtttgcctgctttcgct

SEQ ID NO: 52 - RNA, 2233 nt, Influenza A
  source 1..2233: mol_type = other RNA
agtagaaacaaggtacttttttggacagtatggatagcaaatagtagcattgccacaactatctcaatgcatgtgtgaggaaggagttgaaccaagaagcattaagcaaaacccagggatcattaatcaggcactcctcaattgcttcatatagccccccaagatcaaaggtcccaggttccaggttgtccctaagagcctgaacgataagaagcagttttcttgattcagctgaaaatccttctagttgtggagatgcatacaagctgttgaataccgactttgccaataaagttctgcagaccttcccaatggaaccttcctccactcctttgggggactctccaacgggccatgtttctgatttgttttcaaagaactctttggtcatgtctttctccttgacagaggactcagcttcaatcatactctcgatttgttgaagtgactgaaggaggcaacgcctcatttccatcccccatttcattttaatttttgaggttccatttgtcctcacatacaagaacataggccttgacacatggcctatggcacttcttagaagcatatctcctacctcaagaacacagtacttctcccatttgtgtggttcaagtcttgggtcagtgagggaaaactccatgctcacaaagtttaccacatcggtgtcattccttaagtgggatcttccttttatgatgaaaccgtacaaattggtctttcgccttccctccttagttctacacttgcttatcattggaattaattggaaatcatccattgctgcacaggatgcattaagcaaggcagtattgatgtacacccccttcattatgtattctgtggctctgcaatgagacacctctgctgtgaaataattccttctcatgcttgcaatgtgttcaattggagccgcatcttctccaatctcatcgagctctatccagcttgaatcggtcagttcacatgccttgttgaactcattctgaatccaacttgcaagcgacctcaattctggttcatcactatcatattgcttcaaatcgcctacatctttacaatcgtcaaagtctaccttttctggtgccatgttctcaccaagtgcccactttaactgactcgttttcttcatatttttagtccttggaattttctcctcattctcaatgtcctgcagttctgccagtacttgcttccatgacagaagataatttggatttattcccttttcgtgtggtttaacaacattgggttccttccatccaaagaatgttctcatgcatttgattgcatcatatagcggtatcccctctccctcatgacttgggtcctcaatgcttaattttaaggcatccatcagcaggaatttggaccgctgagaacagggaggcccatccggaagtctaagtggtcgtggtgttgatttcaaaaaaggttcaattctagcatttacttctttggacatttgagaaagcttgccctcaatgtagccgttcggttcgaatccatccacataggctctaaatttttcaaggctggagaagtttggcgggagactttggtcggcaagcttgcgcattgttcctgtgatttcaaatctttcttcaattgtctcttcgcctctctcggactgacgaaaggaatcccagaggcctctgctagccatttcttgtcttatggtgaatagcctggttttgatcctagccctgctttcttcatcgagagtgtagtcggcctttgtggccatttcctccccagtgaatgagaaaatgtggatgtgtgtcttctcagatttaattttattggccttttccagatagtatatgtgaacttctctccttgttactccaatttcgatgaatctattcttcttgtaatcatacaaatctggtagaaactttggtttctcagcccctgtagtgttgcaaatactgtttattactgtccaggctattgtgcgatctcttccctcgattatttcaaatctgtgcttcaaaagtgcatttggatcgccaagttctacgactattgactcgccttgctcatcgatgaagtgaaaatctgaatacatgaagcacacttccaagtgagtgcatattgctgcaaatttgtttgtttcgattttcaggtcctctccatactctttcattgccttttccgcaagctcgacaatcatcggattgaagcattgtcgcacaaaatcttccattttgaatcagtacctgctttcgct

SEQ ID NO: 53 - RNA, 1775 nt, Influenza A
  source 1..1775: mol_type = other RNA
agtagaaacaagggtgtttttccttatatttctgaaatcctaatctcagatgcatattctgcactgcaaagacccattagaacacatccagaaactgattgcccccagggagaccaaaagcaccagtgaactggcgacagttgagtagatcgccagaatctgatacacccccattgattccaatttcactccatctatcttttccctgttcaactttgattcttctgaatattttggataatcataagtcccatttcttacactttccatgcattcattgtcacacttgtggtagaactcaaaacacccatttccgatttctttggcattattctttaattggctttttactttctcgtacagattcttcacatttaagtcatggaaatccaaagtcctttcattttccagtagaactaacaattctgcattatatgtccaaatgtccagaaacccatcatcaacttttttatttaaattttccatccttttttctaagttgttgaattctttacccacagctgtgaattgagtgttcattttctcgataacagagttcaccttgtttgtaatcccgttaatggcattttgtgtgcttttttgatccgctgcatagcctgatccctgttcattctgatgatgataaccataccatccatctatcattccagtccatcccccctcaataaaaccagcaatggctccaaatagacctctgtattgaatggatgggatgtttcttagtcctgtaaccatcctcaatttggtactcctgacatattttgggcactctcctattgtgactgggtgtatattctggaaagggagattgctgtttatagctccctggggtgtttgacacttcgtgttacactcatgcattgacgcgtttgaggtgatgatgccggactcaaaccctctactcagtgcgaaagcataccatggtgctattagattaccagttgcctcaaatattattgtgtctccgggttctagcaaggtccagtaatagttcatcctcccatgttgatctcttactttgggccttgcagctatttccggggtgaatctcctgttataatttgaagacgctacagagacataagcatttccattactatagagactctgttgctcatcactgctagacgggtgatgaacaccccatagtacaaggacttctttccctttattgttcacataggaattggtcagctttgggtatgaatcccccttcttcgtcagccatagcaaatttctgtaaaaactgctttttcccctatgggagcatgatactgttactccgttgaatgtgtggttgggccatgaactttccttgggaaatatttcgaatctttctaatgatgatactgagctcaattgctccctcagttcctcatagtcgatgagatctcctggataacatgctccattctcagagtttggtgtttctacaatgtaggaccatgatctcgctggaagcagtgagtcgcattctggatttcccaagagccatccggtgatgttacatttccccaattgtagtggggctattccttttaatttacatagtttcccgttgtggctgtcttcgagcaggttaacagaatgtgtcactgccacattcttctcgagtattgtgtcaacagtgtcggttgagttgttcgcatggtagcctatacatattgtgtctgcatctgtagctacaaatgcatataacaggaccagtagttttgccttcattttggttgtttttattttcccctgcttttgct

SEQ ID NO: 54 - RNA, 1565 nt, Influenza A
  source 1..1565: mol_type = other RNA
agtagaaacaagggtatttttctttaattgtcgtactcctctgcattgtctccgaagaaataagatccttcattactcatgtcaaaggagggcacgatcgggctcgttgccttttcgtccgagagctcgaagactccccgcccctggaaagacacatcttctggtcttgcactttccatcagccttatgatttcggttctcatgtcagatgttctcccctctgtattcccagtgaatgctgccataatggttggtctgtcaaaagggagatttctctgtactgagaacgtaggttgtatgctgatttggcccgaggaagccctctgttgattggtgttccctccacttctggtccttatggcccagtatctgcttctcagttcaagggtacttgattccatagtctccatgttttcattggaagcaatttgaactcctctagtggaaagcttccctcttgggaccactttcgtccctctgatgaagcttgatactcttagatcttcaaatgcagcagaatggcatgccatccacaccagttgactcttgtgtgctggattctcatttggtctgattaggctgtatacttggctgttttgaagcagtctgaaagggtctattccgactagagagtatccctctctttcaaagtcgtatccactggctacggcagatccatacacacaggcaggcaggcaggacttgtgagcaactgaccctctcaatatgagtgcagaccgtgctaaaaagatgagatcttcgaactcagcatttcctggattccggctctctctcacttgatccaccattgttctttgtgcagctgtttgaaatttccctttgagaatgttgcacattctttcataagcaatccttgttctccgtccattctcacccctccagaagttccgatcattgatcccacgtttgatcattctgatcaattccatcaccattgttccaactcctttgactgcagcacctgcggccccagacctcctagggagggttgaaccctgcatcagtgagcacatcctgggatccattcctgtgcgaacaagagctcttgtcctctggtaagttgcatcattcaaattggagtgccagatcatcatgtgagtcagaccagccgttgcatcgtcaccattattagcttggcgccagattcgtcttatttcttctttgtcataaaggatgagttctctcctccactttccatctactctcctgtatataggtcctccagttttcttaggatctttccccgcactgggatgttcttctagatatttattcctcctctcgtcaaaagcagagagcaccattctctctattgttaagctgttctgaatcagccgtccctcataatcactgagtttaagttcggtgcacatttggatgtagaatcgtccaattccatcaatcatttttccgacagatgctctgatttcagtggcattctggcgttctccatcagtctccatctgttcgtaagatcgtttggtgcctttggtcgccatgatttcgatgtcactctgtgagtgattatctaccctgcttttgct

SEQ ID NO: 55 - RNA, 1409 nt, Influenza A
  source 1..1409: mol_type = other RNA
agtagaaacaaggagttttttgaacaaactacttgtcaatggtgaacggcaactcagcaccgtctggccaagaccaatctacagtatcactattcacaccacaaaaagaaatgatgctcccactagtccagattgcgtcctcctcaggtagccccctgattaattcaacccagaagcaaggcctcatacagtctagccctgttagctcaggatgttgaacgaaacttccgctgtaccctgaccgattagttattgccacaacatcttgtctcatagagaacctactatcagtctctgtccatccattaggatcccaaatcatctcaaacccatgtctggaactgtcacttttagtccttcctatccaaacaccattgccatacttatatgaaaatccctttactccgtttgctccatcagcagacactgggccacagctgcctgttccatctttgggacgcgggttgtcaccgaaaaccccactgcagatgtatcctattttataatctaggttttggtcgaaggacacccatggtcggttcgaaccgtgccaattgtctctgcacacacacatcactttgccggtatcagggtaacaggaacattcctcgtagtgagaattaggtgcattcaactctatcgatttagtaaccttccccttctcgatcttgaaaattttgtacgaggccagcccatcacttgggccatcggtcattatggtaaaacatgaaccatttacacaggtacattcagactcttgtgttctcaatatattcttcctccaactttttatggtttcagttattatgcggttgtattttaatacagccactgctccatcatctggaccagaaattccgattgttagccagcccattccatcatgacatgcacttgctgaccaagcaaccgattcaaaccttgaattgtacggggacggagcttcaccgacagggcagctcattaaggccctataagggcttctgtccttaaaggtcccccttgaatgcttgtcattcagtaaggcgccttgagtcagaaaaaaggtcctgcattccaagtgagaacatgaaataaaaggctctcttatgacaaaaacgtctcctttggaaccaattcttatgccattgtctttgctgtgtatagcccacccacggatgggacaaagagatgaattgccggttaatatcactgaagttgagtcctgcccagcaacaactttataggtaatgctgccttggttgcatattccagtatggttttgatttccggtttgaattgaatggctaatccatattgagattatatttcctatttgcaatattaggctaattattccgactaccatacagattgacccaatggttattattttctggtttggattcatttaaactcctgcttttgct

SEQ ID NO: 56 - RNA, 1027 nt, Influenza A
  source 1..1027: mol_type = other RNA
agtagaaacaaggtagttttttactccagctctatgttgacaaaatgaccatcgtcaacatccacagcattctgctgttcctttcgatattcttccctcatagactctggcactccttccgtagaaggccctcttttcaaaccgtatttaaagcgacgataaatgcatttgaaaaaaagacgatcaagaatccacaatatcaagtgcaagatcccaatgatatttgctgcaatgacgagaggatcacttgaatcgttgcatctgcacccccattcgtttctgataggcctgcaaattttcaagaagatcatcttttagaccagcactggagctaggatgagtcccaacggttctcatcgcctgcaccatttgcctggcctgactagcaatatccatggcctctgctgcttgctcactcgatccagccatttgctccatagccttagctgtagtgctggctagaaccattctgttctcatgtctgattagtggattggttgttgtcaccatttgcctatgagaccgatgctgggagtcagcaatctgttcacaggttgcgcataccaggccaaatgccacttcagtggtcacagcccccatcctgttgtatatgaggcccatacaactggcaagtgcaccagcagaataactgagtgctatttctttggccccatggaatgttatctccctcttaagcttcctatacagtttaactgctttgtccatgttatttggatctccgttcccattaagagcattttggacaaagcgtctacgctgcagtccccgctcactgggcacggtgagcgtgaacacaaatcctaaaatccccttagtcagaggtgacaggattggtcttgtctttagccattccatgagaacctcaagatcggtgttcttccctgcaaagacatcttcaagtctctgtgcgatctcggctttgagggggcctgacgggacgatagagagaacgtacgtttcgacctcggttagaagactcatctttcaatatctacctgcttttgct

SEQ ID NO: 57 - RNA, 890 nt, Influenza A
  source 1..890: mol_type = other RNA
agtagaaacaagggtgttttttattattaaataagctgaaacgagaaagttcttatctcttgctccacttcaagcaatagttgtaaggcttgcataaatgttatttgctcaaaactattctctgttatcttcagtctgtgtctcacttcttcaatcaaccatcttatttcttcaaacttctgacctaattgttcccgccattttccgtttctgttttggagtgagtggaggtctcccattctcattactgcttctccaagcgaatctctgtagagtttcagagactcgaactgtgttattattccattcaagtcctccgatgaggaccccaactgcatttttgacatcctcatcagtatgtcctggaagagagggcagtggtgaaatttcgccaacaattgtcccctcttcggtgaaggcccttagtaatattagagtctccagccggtcaaaaatcacactgaagttcgctttcagtatgatgttcttatccatgatcgcctggtccattctgatacaaagagggcctgccactttctgcttgggcatgagcatgaaccagtgccttgacatttcctcaagagtcatgtcagttaggtagcgcgatgcaggtacagaggccatggtcattttgagtgcctcatcagattcttccttcagaatccgctccactatttgctttccagcacgggtggctgtttcgatgtccagaccaagagtgctgcctcttcctcttagggacttctgatctcggcgaagccgatcaaggaatggggcatcacctagttcttggtctgcaactcttttgcggacatgccaaagaaagcaatctacctgaaagcttgacacagtgtttggatccattatgtctttgtcaccctgcttttgct

SEQ ID NO: 58 - DNA, 12 bp, synthetic construct
  source 1..12: mol_type = other DNA
agtaaaaacagg

SEQ ID NO: 59 - protein, 98 aa, Influenza A
MERIKELRNLMSQSRTREILTKTTVDHMAIIKKYTSGRQEKNPALRMKWMMAMKYPITADKRITEMIPERNEQGQTLWSKMNDAGSDREFGWPSISVE

SEQ ID NO: 60 - protein, 131 aa, Influenza A
MERIKELRNLMSQSRTREILTKTTVDHMAIIKKYTSGRQEKNPALRMKWMMAMKYPITADKRITEMIPERNEQGQTLWSKMNDAGSDRVMVSPLAVTWWNRNGPVTSTVHYPKIYKTYFEKVERLKHGTLG

SEQ ID NO: 61 - protein, 65 aa, Influenza A
MERIKELRNLMSQSRTREILTKTTVDHMAIIKKYTSGRQEKNPALRMKWMMAMKYPVGNETETEL

SEQ ID NO: 62 - protein, 75 aa, Influenza A
MERIKELRNLMSQSRTREILTKTTVDHMAIIKKEKNPALRMKWMMAMKYPITADRNSSILTDSQTATKRIRMAIN

SEQ ID NO: 63 - protein, 117 aa, Influenza A
MERIKELRNLMSQSRTREILTKTTVDHMAIIKKYTSGRQEKNPALRMKWMMAMKYPITADKRITEMIPERNEQGQTLWSKMNDAGSDRVMVSPKRKRNSSILTDSQTATKRIRMAIN

SEQ ID NO: 64 - protein, 4 aa, Influenza A
MDQH

SEQ ID NO: 65 - protein, 100 aa, Influenza A
MERIKELRNLMSQSRTREILTKTTVDHMAIIKKYTSGRQEKNPALRMKWMMAMKYPITADKRITEMIPERNEQGQTLWSKMKNSDGHQLVSNSLKTTLFL

SEQ ID NO: 66 - protein, 131 aa, synthetic construct; identical to SEQ ID NO: 60

SEQ ID NO: 67 - DNA, 569 bp, Influenza A
  source 1..569: mol_type = other DNA
agcgaaagcaggtcaattatattcaatatggaaagaataaaagaactaaggaatctaatgtcgcagtctcgcactcgcgagatactcacaaaaaccaccgtggaccatatggccataatcaagaagtacacatcaggaagacaggagaagaacccagcacttaggatgaaatggatgatggcaatgaaatatccaattacagcagacaagaggataacggaaatgattcctgagagaaatgagcagggacaaactttatggagtaaaatgaatgacgccggatcagaccgagtgatggtatcacctctggctgtgacatggtggaataggaatggaccagtgacaagtacagttcattatccaaaaatctacaaaacttattttgaaaaagtcgaaaggttaaaacatggaaccttaggctaatgtgctaattgggcaaggagacgtggtgttggtaatgaaacggaaacggaactctagcatacttactgacagccagacagcgaccaaaagaattcggatggccatcaattagtgtcgaatagtttaaaaacgaccttgtttctact

SEQ ID NO: 68 - DNA, 569 bp, synthetic construct
  source 1..569: mol_type = other DNA
agtgaaaacaggtcaattatattcaatatggaaagaataaaagaactaaggaatctaatgtcgcagtctcgcactcgcgagatactcacaaaaaccaccgtggaccatatggccataatcaagaagtacacatcaggaagacaggagaagaacccagcacttaggatgaaatggatgatggcaatgaaatatccaattacagcagacaagaggataacggaaatgattcctgagagaaatgagcagggacaaactttatggagtaaaatgaatgacgccggatcagaccgagtgatggtatcacctctggctgtgacatggtggaataggaatggaccagtgacaagtacagttcattatccaaaaatctacaaaacttattttgaaaaagtcgaaaggttaaaacatggaaccttaggctaatgtgctaattgggcaaggagacgtggtgttggtaatgaaacggaaacggaactctagcatacttactgacagccagacagcgaccaaaagaattcggatggccatcaattagtgtcgaatagtttaaaaacgaccttgtttctact

SEQ ID NO: 69 - DNA, 569 bp, synthetic construct
  source 1..569: mol_type = other DNA
agtggaaacaggtcaattatattcaatatggaaagaataaaagaactaaggaatctaatgtcgcagtctcgcactcgcgagatactcacaaaaaccaccgtggaccatatggccataatcaagaagtacacatcaggaagacaggagaagaacccagcacttaggatgaaatggatgatggcaatgaaatatccaattacagcagacaagaggataacggaaatgattcctgagagaaatgagcagggacaaactttatggagtaaaatgaatgacgccggatcagaccgagtgatggtatcacctctggctgtgacatggtggaataggaatggaccagtgacaagtacagttcattatccaaaaatctacaaaacttattttgaaaaagtcgaaaggttaaaacatggaaccttaggctaatgtgctaattgggcaaggagacgtggtgttggtaatgaaacggaaacggaactctagcatacttactgacagccagacagcgaccaaaagaattcggatggccatcaattagtgtcgaatagtttaaaaacgaccttgtttctact

SEQ ID NO: 70 - DNA, 22 bp, synthetic construct
  source 1..22: mol_type = other DNA
acgcgtgatcagcraaagcagg

SEQ ID NO: 71 - DNA, 23 bp, synthetic construct
  source 1..23: mol_type = other DNA
acgcgtgatcagtagaaacaagg